(19) United States
(12) Patent Application Publication
(10) Pub. No.: US 2024/0199734 A1
(43) Pub. Date: Jun. 20, 2024

(54) Method of Treating Ulcerative Colitis with Anti-IL23 Specific Antibody

(71) Applicant: Janssen Biotech, Inc., Horsham, PA (US)

(72) Inventors: Matthew Germinaro, Philadelphia, PA (US); Kuan-hsiang Gary Huang, Horsham, PA (US); Hongyan Zhang, Malvern, PA (US)

(21) Appl. No.: 18/517,248

(22) Filed: Nov. 22, 2023

Related U.S. Application Data
(60) Provisional application No. 63/427,259, filed on Nov. 22, 2022.
(60) Provisional application No. 63/462,682, filed on Apr. 28, 2023.
(60) Provisional application No. 63/541,124, filed on Sep. 28, 2023.

(51) Int. Cl. C07K 16/24; A61K 9/00; A61K 39/00; A61P 1/04

(52) U.S. Cl. CPC C07K 16/244; A61K 9/0019; A61P 1/04; A61K 39/00; A61K 2039/505; C07K 2317/21

(57) ABSTRACT

A method of treating ulcerative colitis in a patient administers an IL-23 specific antibody, e.g., guselkumab, at an initial dose and subsequent doses in order for the patient to respond to the antibody and meet one or more of the clinical endpoints.

### CROSS REFERENCE TO RELATED APPLICATIONS

This application claims priority to U.S. Provisional Patent Application Ser. No. 63/427,259, filed 22 Nov. 2022, 63/462,682, filed 28 Apr. 2023 and 63/541,124, filed 28 Sep. 2023. The entire contents of each of the aforementioned applications is incorporated herein by reference in their entireties.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6766USNP1SEQLIST.xml”, creation date of 24 Oct. 2023, and a size of 11 kilobytes (KB). This sequence listing submitted is part of the specification and is herein incorporated by reference in its entirety.

### FIELD OF THE INVENTION

The present invention is directed to methods of treating ulcerative colitis with an antibody that binds human IL23. In particular, it relates to dosing regimens for administration of an anti-IL23 specific antibody and specific pharmaceutical compositions of an antibody.

### BACKGROUND OF THE INVENTION

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rβ1) chain binds to the p40 subunit of IL-12, providing the primary interaction between IL-12 and its receptor. However, it is IL-12p35 ligation of the second receptor chain, IL-12Rβ2, that confers intracellular signaling (e.g., STAT4 phosphorylation) and activation of the receptor-bearing cell (Presky et al, 1996). IL-12 signaling concurrent with antigen presentation is thought to invoke T cell differentiation towards the T helper 1 (Th1) phenotype, characterized by interferon gamma (IFNγ) production (Trinchieri, 2003). Th1 cells are believed to promote immunity to some intracellular pathogens, generate complement-fixing antibody isotypes, and contribute to tumor immunosurveillance. Thus, IL-12 is thought to be a significant component to host defense immune mechanisms.

It was discovered that the p40 protein subunit of IL-12 can also associate with a separate protein subunit, designated p19, to form a novel cytokine, IL-23 (Oppman et al, 2000). IL-23 also signals through a two-chain receptor complex. Since the p40 subunit is shared between IL-12 and IL-23, it follows that the IL-12Rβ1 chain is also shared between IL-12 and IL-23. However, it is the IL-23p19 ligation of the second component of the IL-23 receptor complex, IL-23R, that confers IL-23 specific intracellular signaling (e.g., STAT3 phosphorylation) and subsequent IL-17 production by T cells (Parham et al, 2002; Aggarwal et al. 2003). Recent studies have demonstrated that the biological functions of IL-23 are distinct from those of IL-12, despite the structural similarity between the two cytokines (Langrish et al, 2005).

Abnormal regulation of IL-12 and Th1 cell populations has been associated with many immune-mediated diseases since neutralization of IL-12 by antibodies is effective in treating animal models of psoriasis, multiple sclerosis (MS), rheumatoid arthritis, inflammatory bowel disease, insulin-dependent (type 1) diabetes mellitus, and uveitis (Leonard et al, 1995; Hong et al, 1999; Malfait et al, 1998; Davidson et al, 1998). However, since these studies targeted the shared p40 subunit, both IL-12 and IL-23 were neutralized in vivo. Therefore, it was unclear whether IL-12 or IL-23 was mediating disease, or if both cytokines needed to be inhibited to achieve disease suppression. Recent studies have confirmed through IL-23p19 deficient mice or specific antibody neutralization of IL-23 that IL-23 inhibition can provide equivalent benefit as anti-IL-12p40 strategies (Cua et al, 2003, Murphy et al, 2003, Benson et al 2004).

Ulcerative colitis is a chronic inflammatory bowel disorder of unknown etiology which involves the surface mucosa, the crypt epithelium, and submucosa of the colon. Ulcerative colitis is most commonly diagnosed in late adolescence and early adulthood, but a diagnosis may occur at any age. Clinically, patients with UC suffer from diarrhea, rectal bleeding, weight loss, abdominal pain, fever, and may also display prominent extraintestinal manifestations, most commonly arthritis. Ulcerative colitis is characterized by a life-long course of remissions and exacerbations, with 15% of patients having an acute attack requiring hospitalization at some time during their illness. In severe UC, the bowel wall may become extremely thin, the mucosa denuded, and the inflammation may extend to the serosa leading to dilatation, toxic megacolon, and subsequent perforation. Within 10 years of diagnosis, approximately 20% of adults with UC were reported to have undergone colectomy. There is a high unmet need for new treatment options in UC that are safe and effective, especially new therapies that can provide improved long-term efficacy (i.e., sustained remission) over currently available therapies.

The pathophysiology of inflammatory bowel disease (IBD) including UC is complex and thought to be multifactorial. The primary aim of pharmacotherapy is to dampen the inflammatory response, thereby relieving symptoms and promoting mucosal healing. The specific goals of IBD treatment include control of symptoms, reduction in need for long-term corticosteroids, prevention of relapses and complications, and minimization of cancer risk (D'Haens G R et al., Future directions in inflammatory bowel disease management. J Crohns Colitis. 2014; 8(8):726-734. EDMS-RIM-476243; Kornbluth A et al., Ulcerative colitis practice guidelines in adults: American College of Gastroenterology, Practice Parameters Committee. Am J Gastroenterol. 2010; 105(3):501-523. Erratum in: Am J Gastroenterol. 2010; 105(3):500. EDMS-ERI-156811382).

The role of IL-23 in promoting intestinal inflammation has been demonstrated in several mouse models where attenuated colitis was exhibited in mice treated with neutralizing anti-IL-23p19 antibodies or in mice with a genetic deletion of the p19 subunit of IL-23. Genome-wide association studies (GWAS) have identified polymorphisms in the IL-23 receptor gene (IL23R) associated with both risk and protection for IBD.

Therefore, there is increasing evidence for the specific role of IL-23 in immune-mediated disease. Neutralization of IL-23 without inhibition of IL-12 pathways could then provide effective therapy of immune-mediated disease with limited impact on important host defense immune mechanism. This would represent a significant improvement over other therapeutic options.

Over the past 20 years, biologic therapies, such as anti-TNFα, IL-12/23 antagonists, and anti-integrins, have revolutionized the clinical management of IBD. Most agents in these classes are approved for the treatment of UC. Within the anti-TNF-α class, infliximab, adalimumab, and golimumab are approved for UC. Ustekinumab, an IL-12/23 antagonist, and vedolizumab, an anti-integrin, are both approved for the treatment of UC. Multiple anti IL-23 agents are currently being evaluated in Phase 3 programs for UC. In addition, two oral small molecule therapies are currently approved in UC, including Janus kinase (JAK) inhibitors and sphingosine-1-phosphate (SlP) receptor modulators.

However, despite the substantial advances conferred with advanced therapies as monotherapies, significant unmet need remains in the treatment of UC. Even with the best available approved therapies, more than half of patients fail to achieve clinical remission after 1 year. Among patients with UC who are clinically asymptomatic, approximately 25% of patients still have endoscopically active disease (Colombel J F et al. Discrepancies between patient-reported outcomes, and endoscopic and histological appearance in UC. Gut 2017; 66: 2063-2068). Thus, it is not surprising that long-term colectomy rates have not declined over a 10-year period (Fumery M et al., Natural history of adult ulcerative colitis in population-based cohorts: A systematic review. Clin Gastroenterol Hepatol 2018; 16:343-56.e3) highlighting the need for more effective therapies and treatment paradigms. The efficacy plateau observed with monotherapies suggests a need for improved treatments that achieve higher rates of long-standing symptomatic and objective remission.

In summary, there remains considerable unmet medical need for new treatment options for IBD and ulcerative colitis, especially therapies with novel mechanisms of action that have the potential to raise the efficacy bar and maximize the proportion of patients who achieve and maintain clinical remission.

### SUMMARY OF THE INVENTION

In a first aspect, the invention concerns a method of treating a subject (patient) suffering from ulcerative colitis comprising administering an anti-IL23 specific antibody (also referred to as IL23p19 or IL23p19 subunit antibody), e.g., guselkumab, to the patient in an initial induction dose from the start of treatment until 4 weeks from the start of treatment, and then administering the anti-IL-23 specific antibody once every 4 weeks thereafter, e.g., a dose at 0, 4, 8, 12, 16, 20, 24, 28, 32, 36, 40, 44 or 48 weeks. In addition, in another embodiment the treatment continues through 96 weeks or longer after the start of treatment.

In one embodiment, the subject receives the anti-IL23 specific antibody (i) at a dose of 200 mg intraveneously initially, 4 weeks after the initial dose intraveneously, 8 weeks after the initial dose intraveneously and 12 weeks after the initial dose intraveneously, and continues with treatment of the anti-IL23 specific antibody, or (ii) at a dose of 400 mg intraveneously initially, 4 weeks after the initial dose intraveneously, 8 weeks after the initial dose intraveneously and 12 weeks after the initial dose intraveneously, and continues with treatment of the anti-IL23 specific antibody, possibly continuing beyond 12 weeks through 24 weeks, 48 weeks, 96 weeks and beyond.

In another aspect, the composition used in the method of the invention comprises a pharmaceutical composition comprising: an anti-IL23 specific antibody.

In an embodiment, ulcerative colitis patients achieve significant improvement in clinical endpoints selected from:
(i) clinical response defined as a decrease from induction baseline in the modified Mayo score by ≥30% and ≥2 points, with either a ≥1-point decrease from baseline in the rectal bleeding subscore or a rectal bleeding subscore of 0 or 1;
(ii) Clinical remission at Week 12, defined as a stool frequency subscore of 0 or 1, a rectal bleeding subscore of 0, and an endoscopy subscore of 0 or 1 with no friability present on the endoscopy, where the stool frequency subscore has not increased from induction baseline.
(iii) Symptomatic remission at Week 12, defined as a stool frequency subscore of 0 or 1 and a rectal bleeding subscore of 0, where the stool frequency subscore has not increased from induction baseline.
(iv) Endoscopic healing at Week 12, defined as an endoscopy subscore of 0 or 1 with no friability present on the endoscopy.
(v) Histologic-endoscopic mucosal healing at Week 12, defined as achieving a combination of histologic healing and endoscopic healing, where histologic healing is defined as neutrophil infiltration in <5% of crypts, no crypt destruction, and no erosions, ulcerations or granulation tissue according to the Geboes grading system.
(vi) Endoscopic normalization at Week 12, defined as an endoscopy subscore of 0 (which requires that no friability is present).

In an embodiment of the invention, a patient who has received the anti-IL23 specific antibody and who is judged not to be in clinical response at Week 12 is treated in an extended induction period receiving subcutaneous anti-IL23 specific antibody at Weeks 12, 16 and 20 and is evaluated for clinical response and other clinical endpoints at Week 24.

In another aspect of the invention the pharmaceutical composition comprises an isolated anti-IL23 specific antibody having the CDR sequences comprising (i) the heavy chain CDR amino acid sequences of SEQ ID NO: 1, SEQ ID NO: 2, and SEQ ID NO: 3; and (ii) the light chain CDR amino acid sequences of SEQ ID NO: 4, SEQ ID NO: 5, and SEQ ID NO: 6, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

Another aspect of the method of the invention comprises administering a pharmaceutical composition comprising an isolated anti-IL-23 specific antibody having the heavy chain variable region amino acid sequence of SEQ ID NO: 7 and the light chain variable region amino acid sequence of SEQ ID NO: 8, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

A further aspect of the method of the invention comprises administering a pharmaceutical composition comprising an isolated anti-IL-23 specific antibody having the heavy chain amino acid sequence of SEQ ID NO: 9 and the light chain amino acid sequence of SEQ ID NO: 10, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

In a still further embodiment, the method of the invention comprises administering a pharmaceutical composition comprising the antibody guselkumab (marketed by Janssen Biotech, Inc as Tremfya®), optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

The details of one or more embodiments of the invention are set forth in the description below. Other features and advantages will be apparent from the following detailed description, figures, and the appended claims.

### BRIEF DESCRIPTION OF THE DRAWINGS

In the Figures:

FIG. 1 is a schematic of the guselkumab Phase 2b/3 clinical development program in ulcerative colitis.

FIG. 2 is a graph showing clinical response at Week 12 and/or 24.

FIG. 3 is a graph showing the primary endpoint of clinical remission at Week 44.

FIG. 4 is a graph showing the primary and major secondary endpoints evaluated at Week 44.

FIG. 5 is a graph showing the proportion of participants in symptomatic remission through Week 44.

FIG. 6 shows a summary of statistical significance of primary and major secondary endpoints per US testing procedure.

### DETAILED DESCRIPTION OF THE PREFERRED EMBODIMENTS

As used herein the method of treatment of a subject suffering from ulcerative colitis comprises administering isolated, recombinant and/or synthetic anti-IL-23 specific human antibodies and diagnostic and therapeutic compositions, methods and devices.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human or humanized, antibodies that have binding specificities for at least two different antigens. In the present case, one of the binding specificities is for at least one IL-23 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-IL-23 specific (also termed IL-23 specific antibodies) (or antibodies to IL-23) useful in the methods and compositions of the present invention can optionally be characterized by high affinity binding to IL-23 and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period.

The term “safe,” as it relates to a dose, dosage regimen, treatment or method with an anti-IL-23 antibody of the present invention (e.g., the anti-IL-23 antibody guselkumab), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator. An adverse event is an untoward medical occurrence in a patient administered a medicinal product. In particular, safe as it relates to a dose, dosage regimen or treatment with an anti-IL-23 antibody of the present invention refers to a relatively low or reduced frequency and/or low or reduced severity of adverse events associated with administration of the antibody if attribution is considered to be possible, probable, or very likely due to the use of the anti-IL-23 antibody.

### Utility

The isolated nucleic acids of the present invention can be used for production of at least one anti-IL-23 antibody or specified variant thereof, which can be used to measure or effect in a cell, tissue, organ or animal (including mammals and humans), to diagnose, monitor, modulate, treat, alleviate, help prevent the incidence of, or reduce the symptoms of ulcerative colitis.

Such a method can comprise administering an effective amount of a composition or a pharmaceutical composition comprising at least one anti-IL-23 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### CITATIONS

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N Y (1989); Harlow and Lane, antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

### Antibodies of the Present Invention—Production and Generation

At least one anti-IL-23 antibody used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N Y (1989); Harlow and Lane, antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

A preferred anti-IL-23 antibody is guselkumab (also referred to as CNTO1959) having the heavy chain variable region amino acid sequence of SEQ ID NO: 7 and the light chain variable region amino acid sequence of SEQ ID NO: 8 and having the heavy chain CDR amino acid sequences of SEQ ID NO: 1, SEQ ID NO: 2, and SEQ ID NO: 3; and the light chain CDR amino acid sequences of SEQ ID NO: 4, SEQ ID NO: 5, and SEQ ID NO: 6. Other anti-IL-23 antibodies have sequences listed herein and are described in U.S. Pat. No. 7,935,344, the entire contents of which are incorporated herein by reference).

Human antibodies that are specific for human IL-23 proteins or fragments thereof can be raised against an appropriate immunogenic antigen, such as an isolated IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Biolnvent, Lund, Sweden; Dyax Corp., Enzon, Affymax/Biosite; Xoma, Berkeley, CA; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (Biolnvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, MA; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g., www.ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research_tools.html; www.whfreeman.com/immunology/CHO5/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wustl.edu/˜hcenter/index.html; www.appliedbiosystems.com; www.nal.usda.gov/awic/pubs/antibody; www.m.ehime-u.ac.jp/˜yasuhito/Elisa.html; www.biodesign.com; www.cancerresearchuk.org; www.biotech.ufl.edu; www.isac-net.org; baserv.uci.kun.nl/˜jraats/links1.html; www.recab.uni-hd.de/immuno.bme.nwu.edu; www.mrc-cpe.cam.ac.uk; www.ibt.unam.mx/vir/V_mice.html; http://www.bioinf.org.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TAHHP.html; www.ibt.unam.mx/vir/structure/stat_aim.html; www.biosci.missouri.edu/smithgp/index.html; www.jerini.de; Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human IL-23 specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human IL-23 specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human IL-23 specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human IL-23 (or anti-IL-23) antibody with improved C1q binding and improved FcγRllIbinding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human IL-23 specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human IL-23 specific antibody of the present invention is expressed in cells that express beta (1,4)-N-acetylglucosaminyltransferase III (GnT III), such that GnT III adds GlcNAc to the human IL-23 antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The anti-IL-23 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-23 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, CA), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge antibody Technologies; 5750373, assigned to Genentech, 5618920, 5595898, 5576195, 5698435, 5693493, 5698417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL23 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL23 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-23 with a wide range of affinities (K_{D}). In a preferred embodiment, a human mAb can optionally bind human IL-23 with high affinity. For example, a human mAb can bind human IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

### Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one anti-IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-23 antibody or variable region; and nucleic acid molecules which comprise a nucleotide sequence substantially different from those described above but which, due to the degeneracy of the genetic code, still encode at least one anti-IL-23 antibody as described herein and/or as known in the art. Of course, the genetic code is well known in the art. Thus, it would be routine for one skilled in the art to generate such degenerate nucleic acid variants that code for specific anti-IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-23 antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

### Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

### Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

### Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (See, e.g., Ausubel, supra; or Sambrook, supra)

### Nucleic Acid Screening and Isolation Methods

A cDNA or genomic library can be screened using a probe based upon the sequence of a polynucleotide used in the method of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides used in the method of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, CA (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids used in the method of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

### Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Agl4 cells (ATCC Accession Number CRL-1851). In a particularly preferred embodiment, the recombinant cell is a P3X63Ab8.653 or a SP2/0-Agl4 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an Antibody

An anti-IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Anti-IL-23 Antibodies.

An anti-IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in the method of the present invention comprise the antibody amino acid sequences disclosed herein encoded by any suitable polynucleotide, or any isolated or prepared antibody. Preferably, the human antibody or antigen-binding fragment binds human IL-23 and, thereby, partially or substantially neutralizes at least one biological activity of the protein. An antibody, or specified portion or variant thereof, that partially or preferably substantially neutralizes at least one biological activity of at least one IL-23 protein or fragment can bind the protein or fragment and thereby inhibit activities mediated through the binding of IL-23 to the IL-23 receptor or through other IL-23-dependent or mediated mechanisms. As used herein, the term “neutralizing antibody” refers to an antibody that can inhibit an IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-23 antibody to inhibit an IL-23-dependent activity is preferably assessed by at least one suitable IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-IL-23 specific antibody can comprise at least one of a heavy or light chain variable region having a defined amino acid sequence. For example, in a preferred embodiment, the anti-IL-23 antibody comprises at least one of a heavy chain variable region, optionally having the amino acid sequence of SEQ ID NO:7 and/or at least one light chain variable region, optionally having the amino acid sequence of SEQ ID NO:8. In an additional preferred embodiment, the anti-IL-23 antibody comprises at least one heavy chain, optionally having the amino acid sequence of SEQ ID NO:9 and/or at least one light chain, optionally having the amino acid sequence of SEQ ID NO:10. Antibodies that bind to human IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-23 with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Amino Acid Codes

The amino acids that make up anti-IL-23 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | THREE |
|---|---|---|---|
| LETTER | LETTER | | NUCLEOTIDE |
| CODE | CODE | NAME | CODON(S) |
| A | Ala | Alanine | GCA, GCC, GCG, |
| | | | GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, |
| | | | GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, |
| | | | CUC, CUG, CUU |
| M | Met | Methionine | AUG |
| N | Asn | Asparagine | AAC, AAU |
| P | Pro | Proline | CCA, CCC, CCG, |
| | | | CCU |
| Q | Gln | Glutamine | CAA, CAG |
| R | Arg | Arginine | AGA, AGG, CGA, |
| | | | CGC, CGG, CGU |
| S | Ser | Serine | AGC, AGU, UCA, |
| | | | UCC, UCG, UCU |
| T | Thr | Threonine | ACA, ACC, ACG, |
| | | | ACU |
| V | Val | Valine | GUA, GUC, GUG, |
| | | | GUU |
| W | Trp | Tryptophan | UGG |
| Y | Tyr | Tyrosine | UAC, UAU |

An anti-IL-23 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL-23 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-23 specific antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL-23 antibodies can include, but are not limited to, at least one portion, sequence or combination selected from 5 to all of the contiguous amino acids of at least one of SEQ ID NOS: 1, 2, 3, 4, 5, and 6.

IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-23 antibody can further optionally comprise a polypeptide of at least one of 70-100% of 5, 17, 10, 11, 7, 9, 119, or 108 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing:Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, MD).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:
(1) Algorithm: Needleman and Wunsch, J. Mol Biol. 48:443-453 (1970) Comparison matrix: BLOSSUM62 from Hentikoff and Hentikoff, Proc. Natl. Acad. Sci, USA. 89:10915-10919 (1992)
Gap Penalty: 12
Gap Length Penalty: 4
   A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:
(1) Algorithm: Needleman and Wunsch, J. Mol Biol. 48:443-453 (1970)
Comparison matrix: matches=+10, mismatch=0
Gap Penalty: 50
Gap Length Penalty: 3
Available as: The “gap” program from Genetics Computer Group, Madison Wis. These are the default parameters for nucleic acid sequence comparisons.

By way of example, a polynucleotide sequence may be identical to another sequence, that is 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the sequence by the numerical percent of the respective percent identity (divided by 100) and subtracting that product from the total number of nucleotides in the sequence, or:
n.sub.n.ltorsim.x.sub.n-(x.sub.n.y),
   wherein n.sub.n is the number of nucleotide alterations, x.sub.n is the total number of nucleotides in sequence, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the SEQ ID NOs above may create nonsense, missense or frameshift mutations in this coding sequence and thereby alter the polypeptide encoded by the polynucleotide following such alterations. Similarly, a polypeptide sequence may be identical to the reference sequence of the SEQ ID NOs above, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the SEQ ID NOs above by the numerical percent of the respective percent identity (divided by 100) and then subtracting that product from the total number of amino acids in the SEQ ID NOs above, or:
n.sub.a.ltorsim.x.sub.a-(x.sub.a.y),
   wherein n.sub.a is the number of amino acid alterations, x.sub.a is the total number of amino acids in the SEQ ID NOs above, and y is, for instance 0.70 for 70%, 0.80 for 80%, 0.85 for 85% etc., and wherein any non-integer produce of x.sub.a and y is rounded down to the nearest integer prior to subtracting it from x.sub.a.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-23 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, CA (1996)). An activating group can be bonded directly to the organic group (e.g., hydrophilic polymer, fatty acid, fatty acid ester), or through a linker moiety, for example, a divalent C₁-C₁₂ group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, CA (1996).

The method of the present invention also uses an anti-IL-23 antibody composition comprising at least one, at least two, at least three, at least four, at least five, at least six or more anti-IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-23 antibody amino acid sequence selected from the group consisting of 70-100% of the contiguous amino acids of the SEQ ID NOs above, or specified fragments, domains or variants thereof. Preferred anti-IL-23 antibody compositions include at least one or two full length, fragments, domains or variants as at least one CDR or LBP containing portions of the anti-IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

### Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, NJ; Pharmcotherapy Handbook, Wells et al., ed., Appleton & Lange, Stamford, CT, each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one corticosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocortisone valerate, mometasone furoate, and triamcinolone acetonide. (See, e.g., pp. 1098-1136 of Nursing 2001 Drug Handbook.)

Anti-IL-23 antibody compositions can further comprise at least one of any suitable and effective amount of a composition or pharmaceutical composition comprising at least one anti-IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-40 et al. (e.g., IL-1, IL-2, etc.). Suitable dosages are well known in the art. See, e.g., Wells et al., eds., Pharmacotherapy Handbook, 2^{nd} Edition, Appleton and Lange, Stamford, C T (2000); PDR Pharmacopoeia, Tarascon Pocket Pharmacopoeia 2000, Deluxe Edition, Tarascon Publishing, Loma Linda, CA (2000), each of which references are entirely incorporated herein by reference.

Anti-IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, PA) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-23 antibody, portion or variant compositions according to the invention are known in the art, e.g., as listed in “Remington: The Science & Practice of Pharmacy,” 19^{th} ed., Williams & Williams, (1995), and in the “Physician's Desk Reference,” 52^{nd} ed., Medical Economics, Montvale, NJ (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-23 specific antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-23 specific antibody, and a second vial comprising an aqueous diluent of prescribed buffer or preservative, wherein said packaging material comprises a label that instructs a patient to reconstitute the anti-IL-23 specific antibody in the aqueous diluent to form a solution that can be held over a period of twenty-four hours or greater.

The anti-IL-23 specific antibody used in accordance with the present invention can be produced by recombinant means, including from mammalian cell or transgenic preparations, or can be purified from other biological sources, as described herein or as known in the art.

The range of the anti-IL-23 specific antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-23 specific antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-23 specific antibody and preservative in an aqueous diluent is carried out using conventional dissolution and mixing procedures. To prepare a suitable formulation, for example, a measured amount of at least one anti-IL-23 specific antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D® Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject©, Smartject® e.g., as made or developed by Becton Dickensen (Franklin Lakes, NJ, www.bectondickenson.com), Disetronic (Burgdorf, Switzerland, www.disetronic.com; Bioject, Portland, Oregon (www.bioject.com); National Medical Products, Weston Medical (Peterborough, UK, www.weston-medical.com), Medi-Ject Corp (Minneapolis, MN, www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products may include packaging material. The packaging material provides, in addition to the information required by the regulatory agencies, the conditions under which the product can be used. The packaging material of the present invention provides instructions to the patient, as applicable, to reconstitute the at least one anti-IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g., 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-23 specific mAbs bind to similar or different epitopes and/or compete with each other. Abs are individually coated on ELISA plates. Competing mAbs are added, followed by the addition of biotinylated hrIL-23. For positive control, the same mAb for coating may be used as the competing mAb (“self-competition”). IL-23 binding is detected using streptavidin. These results demonstrate whether the mAbs recognize similar or partially overlapping epitopes on IL-23.

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(β-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating ulcerative colitis, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, C T (2000); PDR Pharmacopoeia, Tarascon Pocket Pharmacopoeia 2000, Deluxe Edition, Tarascon Publishing, Loma Linda, CA (2000); Nursing 2001 Handbook of Drugs, 21^{st} edition, Springhouse Corp., Springhouse, PA, 2001; Health Professional's Drug Guide 2001, ed., Shannon, Wilson, Stang, Prentice-Hall, Inc, Upper Saddle River, NJ, each of which references are entirely incorporated herein by reference.

### Therapeutic Treatments

Typically, treatment of ulcerative colitis is affected by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-23 specific antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of an anti-IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Example 1—QUASAR UC Study

Title: A Phase 2b/3, Randomized, Double-blind, Placebo-controlled, Parallel-group, Multicenter Protocol to Evaluate the Efficacy and Safety of Guselkumab in Participants with Moderately to Severely Active Ulcerative Colitis

### Primary Objective:

The primary objective is to evaluate the clinical efficacy and safety of guselkumab as induction therapy in participants with moderately to severely active UC.

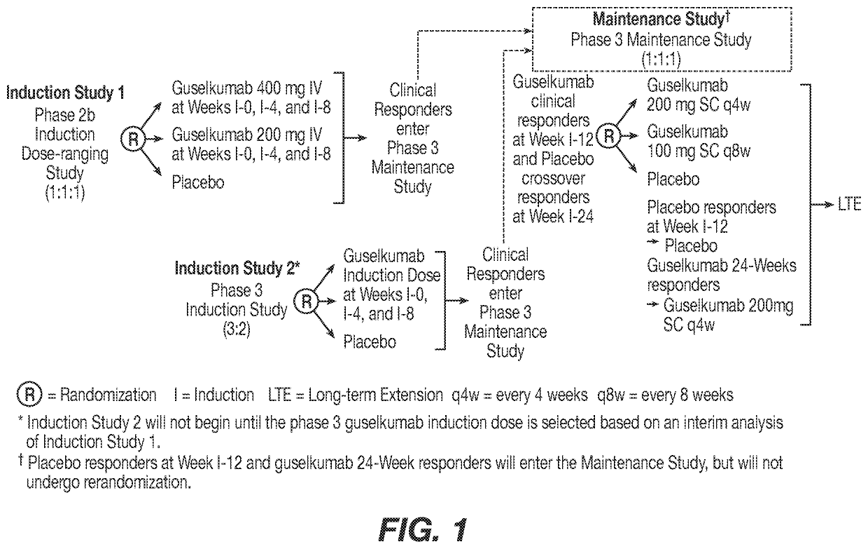

The guselkumab Phase 2b/3 clinical development program in ulcerative colitis (QUASAR) is comprised of 3 separate studies: a Phase 2b induction dose-ranging study (Induction Study 1), a Phase 3 induction study (Induction Study 2), and a Phase 3 maintenance study (Maintenance Study). A schematic of the program is shown in FIG. 1.

### Phase 2b Induction Study Trial Design:

The Phase 2b Induction Study is a randomized, double-blind, placebo-controlled, parallel-group, multicenter study.

The target population consists of participants with moderately to severely active ulcerative colitis (UC) who have demonstrated an inadequate response or failure to tolerate conventional (i.e., 6-MP, AZA, or corticosteroids) or advanced therapy (i.e., TNFα antagonists, vedolizumab, or tofacitinib). At Week I-0, participants must have moderately to severely active UC, defined as a modified Mayo score of 5 to 9, inclusive, Mayo rectal bleeding subscore ≥1 and a Mayo endoscopy subscore ≥2, using the Mayo endoscopy subscore obtained during the central review of the video endoscopy. Note that the program also allows for the enrollment of participants with a modified Mayo score of 4, which is capped at ≤5% of the total population. The protocol was amended per health authority's recent feedback that the target population would be based on only participants with modified Mayo score of 5 to 9.

Treatment allocation: Participants were randomized at Week I-0 in a 1:1:1 ratio to 1 of 3 treatment groups, using permuted block randomization with ADT-Failure status (i.e., inadequate response or failure to tolerate TNFα antagonists, vedolizumab, or tofacitinib) (Yes/No), region (Eastern Europe, Asia, or rest of world), and concomitant use of corticosteroids at baseline (Yes/No) as stratification variables:
Group 1: Placebo IV (Weeks I-0, I-4, and I-8)
Group 2: Guselkumab 200 mg IV (Weeks I-0, I-4, and I-8)
Group 3: Guselkumab 400 mg IV (Weeks I-0, I-4, and I-8)

Treatment duration: the main part of this study is 12 weeks.

Primary endpoint: The primary endpoint was clinical response at Week I-12, defined as a decrease from induction baseline in the modified Mayo score by ≥30% and ≥2 points, with either a ≥1-point decrease from baseline in the rectal bleeding subscore or a rectal bleeding subscore of 0 or 1.

### Major Secondary Endpoints:

Clinical remission at Week I-12, defined as a stool frequency subscore of 0 or 1, a rectal bleeding subscore of 0, and an endoscopy subscore of 0 or 1 with no friability present on the endoscopy, where the stool frequency subscore has not increased from induction baseline.
Symptomatic remission at Week I-12, defined as a stool frequency subscore of 0 or 1 and a rectal bleeding subscore of 0, where the stool frequency subscore has not increased from induction baseline.
Endoscopic healing at Week I-12, defined as an endoscopy subscore of 0 or 1 with no friability present on the endoscopy.
Histologic-endoscopic mucosal healing at Week I-12, defined as achieving a combination of histologic healing and endoscopic healing, where histologic healing is defined as neutrophil infiltration in <5% of crypts, no crypt destruction, and no erosions, ulcerations or granulation tissue according to the Geboes grading system.
Endoscopic normalization at Week I-12, defined as an endoscopy subscore of 0 (which requires that no friability is present).
IBDQ remission at Week I-12.
Fatigue response at Week I-12.

At Week I-12, all participants will be evaluated for clinical response. Further study intervention administration will be determined by the participant's clinical response status (using the Mayo endoscopy subscore assigned by the local endoscopist) at Week I-12, as follows:

Guselkumab clinical responders and placebo clinical responders at Week I-12 will enter the Maintenance Study.

Participants initially randomized to placebo who are not in clinical response at Week I-12 will then crossover to guselkumab and receive 3 doses of guselkumab 200 mg IV at Weeks 1-12, I-16, and I-20.

Participants initially randomized to guselkumab who are not in clinical response at Week I-12 will then receive 3 doses of guselkumab 200 mg SC at Weeks 1-12, I-16, and I-20.

To maintain the blind, both IV and SC administrations will be given to all participants who are not in clinical response at Week I-12.

At Week I-24, participants who were not in clinical response at Week I-12 will be re-evaluated for clinical response (clinical response status will be based on the Mayo endoscopy subscore assigned by the local endoscopist). In addition to guselkumab clinical responders and placebo clinical responders at Week I-12, the following participants from Induction Study 1 will enter the Maintenance Study:

Placebo crossover responders: Participants initially randomized to placebo who are not in clinical response at Week I-12 who then crossover to guselkumab induction 200 mg IV treatment and achieve clinical response at Week I-24.

Guselkumab 24-Week responders: Participants initially randomized to guselkumab who are not in clinical response at Week I-12 who then receive 3 doses of guselkumab 200 mg SC and achieve clinical response at Week I-24.

Participants who are not in clinical response at Week I-24 will not receive further study intervention and should have a safety follow-up visit approximately 12 weeks after their last dose of study intervention.

All UC-specific medical therapies (i.e., oral 5-aminosalicylic [5-ASA]compounds, oral corticosteroids, 6-MP, AZA, or MTX) must be maintained at a stable dose through to the end of Induction Study 1 and can only be discontinued or reduced in dose if investigator judgment requires it because of toxicity or medical necessity. The initiation or increase in dose of UC-specific therapies (or any restricted/prohibited medication or therapy) during Induction Study 1 will prohibit a participant from entering the Maintenance Study. Efficacy, PK parameters, biomarkers, and safety will be assessed according to the SoA.

An interim analysis of the first 150 randomized participants who have completed the Week I-12 visit or have terminated study participation prior to Week I-12 will be performed. The purpose of this interim analysis is to select a single induction dose for confirmatory evaluation in the Phase 3 induction study (Induction Study 2). A Dose Selection Committee, composed of sponsor management representatives from Clinical, Safety, Biostatistics, and Clinical Pharmacology, who are not associated with study conduct, will be responsible for selecting the induction dose of guselkumab to be evaluated in Induction Study 2. While the data from the first 150 randomized participants is being evaluated, participants will continue to be enrolled in Induction Study 1, up to a maximum of 390 participants. Once the induction dose selection has occurred, participants will begin randomization into Induction Study 2.

In Induction Study 2, participants will be randomized in a 3:2 ratio to guselkumab or placebo administered at Weeks I-0, I-4, and I-8. Induction Study 2 targets a sample size of at least 560 randomized participants with a modified Mayo score of 5 to 9. Selection of the guselkumab induction dose for Induction Study 2 will be based on an interim analysis of Induction Study 1. Participants will be allocated to an intervention group using permuted block randomization stratified by ADT-Failure status (ie, inadequate response or failure to tolerate TNFα antagonists, vedolizumab, or tofacitinib) (Yes/No), region (Eastern Europe, Asia, or rest of world), and concomitant use of corticosteroids at baseline (Yes/No). At Week I-12, all participants will be evaluated for clinical response. Similar to the approach outlined in Induction Study 1, further study intervention administration will be determined by the participant's clinical response status (using the Mayo endoscopy subscore assigned by the local endoscopist) at Week I-12, as follows:

Guselkumab clinical responders and placebo clinical responders at Week I-12 will enter the Maintenance Study.

Participants initially randomized to placebo who are not in clinical response at Week I-12 will then crossover to guselkumab and receive 3 doses of guselkumab IV treatment (i.e., induction dose selected based on Induction Study 1 interim analysis) at Weeks I-12, I-16, and I-20.

Participants initially randomized to guselkumab who are not in clinical response at Week I-12 will then receive 3 doses of guselkumab 200 mg SC at Weeks 1-12, 1-16, and I-20.

To maintain the blind, both IV and SC administrations will be given to all participants who are not in clinical response at Week I-12.

At Week I-24, participants who were not in clinical response at Week I-12 will be re-evaluated for clinical response (clinical response status will be based on the Mayo endoscopy subscore assigned by the local endoscopist). In addition to guselkumab clinical responders and placebo clinical responders at Week I-12, the following participants from Induction Study 2 will enter the Maintenance Study:

Placebo crossover responders: Participants initially randomized to placebo who are not in clinical response at Week I-12 who then crossover to guselkumab induction IV dose treatment and achieve clinical response at Week I-24.

Guselkumab 24-Week responders: Participants initially randomized to guselkumab who are not in clinical response at Week I-12 who then receive 3 doses of guselkumab 200 mg SC and achieve clinical response at Week I-24.

Participants who are not in clinical response at Week I-24 will not receive further study intervention and should have a safety follow-up visit approximately 12 weeks after their last dose of study intervention.

All UC-specific medical therapies (ie, oral 5-ASA compounds, oral corticosteroids, 6-MP, AZA, or MTX) must be maintained at a stable dose through to the end of Induction Study 2 and can only be discontinued or reduced in dose if investigator judgment requires it because of toxicity or medical necessity. The initiation or increase in dose of UC-specific therapies (or any restricted/prohibited medication or therapy) during Induction Study 2 will prohibit a participant from entering the Maintenance Study.

### Results

### Topline Phase 2b Results Summary

A total of 327 participants were randomized and dosed in 141 sites across 27 countries. The majority (47.4%) of participants were from Eastern Europe, with the remaining participants distributed across Asia (23.2%), and Rest of World (29.4%). Of note, there was 1 participant who was randomized but never received study intervention.

Among randomized and treated participants, 313 (95.7%) participants had a modified Mayo score of 5 to 9 (target population to be used for the efficacy and safety analyses below).

Disposition and Baseline Characteristics for the Full Analysis Set (n=313):

Overall, 9 (2.9%) participants discontinued study treatment prior to Week I-12. There were 5 (4.8%) discontinuations in the placebo group, 3 (3.0%) in the 200 mg IV guselkumab group, and 1 (0.9%) in 400 mg IV guselkumab group. For the placebo group, 4 of the 5 discontinuations were due to reasons indicative of lack of efficacy. The most common reasons for treatment discontinuation prior to Week I-12 were adverse events due to worsening UC (1.0%) and withdrawal by subject (1.0%). No participants discontinued study agent prior to Week I-12 due to COVID-19 related reasons.

The majority of participants were white (71.6%), and 59.1% of the participants were male. The mean age was 41.6 years (range 18 to 84 years). A total of 147 (47.0%) participants had a history of advanced therapy (ADT) failure; 166 (53.0%) had failed conventional therapy but not advanced therapy and the majority (93.4%) of these participants were ADT-naïve. Approximately 40% of participants were receiving corticosteroids (including budesonide and beclomethasone dipropionate) at baseline and 21.7% were receiving immunomodulators (6-mercaptopurine, azathioprine, or methotrexate). Approximately 90% of participants had a history of an inadequate response, intolerance, or dependence to corticosteroids and/or 6-MP/AZA.

The population in this study is representative of a population with moderately to severely active UC. The mean duration of UC disease was 7.55 years. The median Mayo score was 9.0 (mean=9.2), the median Modified Mayo score was 7.0 (mean=7.0), the median fecal calprotectin was 1564.0 mg/kg, and the median C-reactive protein (CRP) concentration was 4.6 mg/L. At baseline, 48.9% of participants had extensive disease, 82.4% of participants had moderate UC (i.e., a Mayo score ≥6 and ≤10) and 17.6% had severe disease (Mayo score >10), and 30% of participants had an endoscopy subscore of 2 (i.e., moderate disease) and 70% of participants had an endoscopy subscore of 3 (i.e., severe disease).

The baseline demographics (including region), disease characteristics, concomitant UC medications, and UC medication history, were generally well-balanced across the treatment groups. However, a higher proportion of participants in the 400 mg IV guselkumab group had extensive disease (55.1%) compared to the placebo group (43.8%) and 200 mg IV guselkumab group (47.5%).

### Efficacy Endpoints Summary:

Guselkumab induction treatment (at both doses evaluated) resulted in significantly higher rates of clinical response at Week I-12 (primary endpoint).
Relative to placebo, guselkumab induction treatment (at both doses evaluated) also resulted in higher rates of clinical remission, symptomatic remission, endoscopic healing, histologic-endoscopic mucosal healing, and endoscopic normalization at Week I-12.

### Primary Endpoint:

Based on the primary analysis of clinical response at Week I-12, a significantly greater proportion of participants in the 200 mg IV and 400 mg IV guselkumab groups were in clinical response at Week I-12 compared with the placebo group. The study is considered to be a positive study.

### Major Secondary Endpoints:

A greater proportion of participants in the 200 mg IV and 400 mg IV guselkumab groups achieved clinical remission, symptomatic remission, endoscopic healing, and endoscopic normalization at Week I-12 compared with the placebo group.

Separation between the guselkumab treatment groups and placebo for symptomatic remission was observed as early as 4 weeks after the first dose and continued through Week I-12.

Safety Data through Week I-12 Based on the Safety Analysis Set:
The average duration of follow-up was similar across treatment groups.
The proportions of participants reporting 1 or more adverse events (AEs) in the guselkumab groups were not higher compared with the placebo group, and clinically meaningful differences in AE rates were not observed among the guselkumab groups.
   The system organ classes (SOCs) with the most frequently reported AEs were Infections and Infestations (11.5% Combined guselkumab; 10.5% Placebo), Gastrointestinal Disorders (9.6% Combined guselkumab; 17.1% Placebo) and Blood and lymphatic system disorders (9.6% Combined guselkumab; 14.3% Placebo).
   The most common preferred terms (PTs) in the combined guselkumab group were Anaemia (7.2%), Headache (4.3%), and COVID-19 (3.8%).
   The PT of Anaemia was comparable across treatment groups (9.5% in the placebo group, 6.9% in the guselkumab 200 mg IV group, and 7.5% in the guselkumab 400 mg IV group).
The proportions of participants reporting 1 or more serious AEs were not higher in the guselkumab treatment groups compared with the placebo group. The majority of SAEs were exacerbations of UC. No deaths were reported.
AEs leading to discontinuation of treatment were low and comparable across all treatment groups (2 in the placebo group, 1 in the guselkumab 200 mg IV group, and 0 in the guselkumab 400 mg IV group) (Table 2).
The proportions of participants reporting AEs of infections as identified by the investigator were comparable among treatment groups (11.4% in the placebo group, 12.9% in the guselkumab 200 mg IV group, and 8.4% in the guselkumab 400 mg IV group).
   Two serious infections were observed, and both occurred in the placebo group.
   No cases of active TB were reported.
   No opportunistic infections were reported.
There were no cases of malignancy.
Hepatic laboratory values were similar across treatment groups through Week I-12. Transaminase elevations were low grade (Common Terminology Criteria for Adverse Events [CTCAE] Grade 1). No cases met Hy's law criteria (i.e., total bilirubin >2×the upper limit of normal [ULN] and either aspartate aminotransferase [AST] or ALT≥3×ULN at the same timepoint).

The incidence of total WBC count decrease was higher in the guselkumab treatment groups compared to placebo through Week I-12. All total WBC abnormalities were CTCAE Grades 1 or 2.

### Week 24 Results for Patients not in Clinical Response at Week 12

The QUASAR Induction Study 1 (NCT04033445) is a Phase 2b study to evaluate guselkumab (GUS) therapy in patients with ulcerative colitis (UC) who had an inadequate response or intolerance to conventional therapy (i.e., thiopurines or corticosteroids) or advanced therapy (i.e., tumor necrosis factor alpha antagonists, vedolizumab, or tofacitinib). Patients who were in clinical response at Week 12 after IV induction entered the maintenance study and those who were not in clinical response received treatment in an extended induction period.

### Methods:

Patients included had moderately to severely active UC (modified Mayo score of 5 to 9 with a Mayo rectal bleeding subscore ≥1 and a Mayo endoscopy subscore ≥2). Patients were randomized 1:1:1 to IV GUS 200 mg, 400 mg, or placebo (PBO) at Weeks 0, 4, and 8. At Week 12, patients who were not in clinical response to IV induction received SC treatment (PBO IV→GUS 200 mg IV; GUS 200 mg IV→GUS 200 mg SC; GUS 400 mg IV→GUS 200 mg SC at Weeks 12, 16, and 20) and were evaluated at Week 24.

Three hundred thirteen patients were randomized at baseline. Demographic and disease characteristics at baseline were similar among the treatment groups (mean age, 41.6 yrs; male 59.1%, mean UC duration, 7.55 yrs; mean Mayo score, 9.2; endoscopy subscore of 3 indicating severe disease, 70%; oral corticosteroid use, 39.9%) and about 50% had a prior inadequate response or intolerance to advanced UC therapy.

At Week 12, clinical response was achieved by 27.6% (29/105) of patients randomized to PBO IV and by 61.4% (62/101) and 60.7% (65/107) of patients randomized to GUS 200 mg and GUS 400 mg IV, respectively, at baseline. Of the patients in the GUS groups who were not in clinical response at Week 12, 54.3% (19/35) who received GUS 200 mg IV→200 mg SC and 50.0% (19/38) who received GUS 400 mg IV→200 mg SC achieved clinical response at Week 24. Tables 16-21 show the number of subjects in clinical remission and clinical response at Week 24. Clinical response at Wk 12 or 24 was achieved by 80.2% of patients who received GUS 200 mg IV→200 mg SC and 78.5% of patients who received GUS 400 mg IV→200 mg SC.

### Conclusion

Patients who did not achieve clinical response to GUS IV induction at Wk 12 showed benefit at Wk 24 after receiving three SC doses of GUS, with approximately 80% of patients who received GUS IV or GUS IV→SC achieving clinical response at Wks 12 or 24. No new safety concerns for GUS were identified.

### Example 2—Phase 3 Study Results

### Phase 3 Induction Study Trial Design:

The Phase 3 Induction Study is a randomized, double-blind, placebo-controlled, parallel-group, multicenter study. The target population consists of participants with moderately to severely active ulcerative colitis (UC) who have demonstrated an inadequate response or failure to tolerate conventional (i.e., 6-mercaptopurine [6-MP], azathioprine [AZA], or corticosteroids) or advanced therapy (ADT; i.e., tumor necrosis factor-alpha [TNFα] antagonists, vedolizumab, or tofacitinib). At Week I-0, participants must have moderately to severely active UC, defined as a modified Mayo score of 5 to 9, inclusive, Mayo rectal bleeding subscore ≥1 and a Mayo endoscopy subscore ≥2, using the Mayo endoscopy subscore obtained during the central review of the video endoscopy. The QUASAR program also allowed for the enrollment of participants with a modified Mayo score of 4, which was capped at ≤5% of the total population. The protocol was amended per health authority feedback so that the target population should be based on only participants with a modified Mayo score of 5 to 9.

Treatment allocation: Participants were randomized at Week I-0 in a 3:2 ratio to guselkumab or placebo, using permuted block randomization with ADT-failure status (i.e., inadequate response or failure to tolerate TNFα antagonists, vedolizumab, or tofacitinib) (Yes/No), region (Eastern Europe, Asia, or rest of world), and concomitant use of corticosteroids at baseline (Yes/No) as stratification variables:
Group 1: Placebo IV (Weeks I-0, I-4, and I-8)
Group 2: Guselkumab 200 mg IV (Weeks I-0, I-4, and I-8)

Treatment duration: the main part of this study is 12 weeks.

This report provides the results of the primary and major secondary endpoints at Week I-12, as well as safety through Week I-12.

Primary endpoint: The primary endpoint was clinical remission at Week I-12, defined as a Mayo stool frequency subscore of 0 or 1 and not increased from baseline, a Mayo rectal bleeding subscore of 0, and a Mayo endoscopy subscore of 0 or 1 with no friability present on the endoscopy.

### Major Secondary Endpoints:

Symptomatic remission at Week I-12: a stool frequency subscore of 0 or 1 and not increased from baseline, and a rectal bleeding subscore of 0.
Endoscopic healing at Week I-12: an endoscopy subscore of 0 or 1 with no friability present on the endoscopy.
Clinical response at Week I-12: a decrease from baseline in the modified Mayo score by ≥30% and ≥2 points, with either a ≥1-point decrease from baseline in the rectal bleeding subscore or a rectal bleeding subscore of 0 or 1.
Symptomatic remission at Week I-4.
Inflammatory Bowel Disease Questionnaire (IBDQ) remission at Week I-12: total IBDQ score ≥170.
Histologic-endoscopic mucosal healing at Week I-12: achieving a combination of histologic healing and endoscopic healing, where endoscopic healing is defined above and histologic healing is defined as neutrophil infiltration in <5% of crypts, no crypt destruction, and no erosions, ulcerations or granulation tissue according to the Geboes grading system.
Fatigue response at Week I-12: ≥7-point improvement in PROMIS-Fatigue short form 7a.
Symptomatic remission at Week I-2.
Endoscopic normalization at Week I-12: an endoscopy subscore of 0

In 90% power for all the major secondary endpoints except for the endpoints of symptomatic remission at Week I-2 and endoscopic normalization at Week I-12.

### Statistical Considerations:

Analysis set for efficacy: The Full Analysis Set includes all randomized participants with a modified Mayo score of 5 to 9 who received at least 1 (partial or complete) dose of study intervention. Participants were analyzed according to their randomized or assigned study intervention regardless of the study intervention they actually received.
Analysis set for safety: The Safety Analysis Set includes all randomized participants with a modified Mayo score of 5 to 9 who received at least 1 (partial or complete) dose of study intervention. The results based on the Safety All Treated Analysis Set, which includes all randomized participants (regardless of modified Mayo score) who received at least 1 (partial or complete) dose of study intervention, are also provided. Participants were analyzed according to the study intervention they actually received.

Intercurrent events (ICEs) were used in the analysis of the efficacy endpoints. In particular, participants who had a UC-related surgery (an ostomy or colectomy), a prohibited change in UC medication, or discontinued study intervention due to reasons other than coronavirus 19 (COVID19) related reasons (excluding COVID-19 infection) or the regional crisis in Russia and Ukraine, including lack of efficacy or an AE of worsening UC, prior to the analysis timepoint were considered not to have met the endpoint for binary endpoints (i.e., composite strategy). For participants who had discontinued study intervention due to COVID-19 related reasons (excluding COVID-19 infection) or the regional crisis in Russia and Ukraine prior to the analysis timepoint, their observed values will be used, if available (i.e., treatment policy strategy).

Comparisons were based on the guselkumab group versus the placebo group. For the primary and major secondary endpoints, the p-values were based on a Cochran-Mantel-Haenszel (CMH) test (2-sided) stratified by ADT-failure status (Yes/No) and concomitant use of corticosteroids at baseline (Yes/No). The 95% confidence intervals were based on the Wald statistic with CochranMantel-Haenszel weight. For these endpoints, participants who had missing data at the analysis timepoint (after accounting for the intercurrent event strategies) were considered non-responders at that timepoint.

### Topline Results Summary

A total of 735 participants were randomized and dosed across 240 sites in 32 countries. The majority (41.5%) of participants were from Eastern Europe, with the remaining participants distributed across Asia (20.5%), and Rest of World (38.0%). Of note, there was 1 participant who was randomized to the placebo group but never received study intervention. Among randomized and treated participants, 701 (95.4%) participants had a modified Mayo score of 5 to 9 (target population to be used for the efficacy and safety analyses below).
Disposition and baseline characteristics for the Full Analysis Set (n=701):

Overall, 42 (6.0%) participants discontinued study intervention prior to Week I-12: 24 (8.6%) discontinuations in the placebo group and 18 (4.3%) in the 200 mg IV guselkumab group. The most common reasons for study intervention discontinuation prior to Week I-12 were adverse events (2.4%; 1.4% due to worsening UC), and withdrawal by participant (2.3%).

The majority of participants were white (72.5%), and 56.9% of the participants were male. The mean age was 40.5 years (range 18 to 79 years). A total of 344 (49.1%) participants had a history of ADT failure; 357 (50.9%) had failed conventional therapy but not advanced therapy (“ADT nonfailure”) and the majority (95.0%) of these participants were ADT-naïve. Approximately 43.1% of participants were receiving corticosteroids (including budesonide and beclomethasone dipropionate) at baseline and 20.5% were receiving immunomodulators (6-MP, AZA, or methotrexate). A total of 93.2% of participants had a history of an inadequate response, intolerance, or dependence to corticosteroids and/or 6-MP/AZA.

The enrolled population in this study is representative of a population with moderately to severely active UC. The mean duration of UC disease was 7.27 years. The median Mayo score was 9.0 (mean=9.1), the median Modified Mayo score was 7.0 (mean=6.9), the median fecal calprotectin was 1641.0 mg/kg, and the median C-reactive protein (CRP) concentration was 4.2 mg/L. At baseline, 47.8% of participants had extensive disease, 82.2% of participants had moderate UC (i.e., a Mayo score ≥6 and ≤10) and 17.8% had severe disease (Mayo score >10), and 32.1% of participants had an endoscopy subscore of 2 (i.e., moderate disease) and 67.9% of participants had an endoscopy subscore of 3 (i.e., severe disease).

The baseline demographics (including region), disease characteristics, concomitant UC medications, and UC medication history, were generally well-balanced across the treatment groups.

### Efficacy Endpoints Summary:

Based on the pre-specified multiple testing procedures, guselkumab induction treatment resulted in a significantly greater proportion of participants in clinical remission at Week I-12 (primary endpoint; 22.6%) compared with placebo (7.9%; adjusted treatment difference: 14.9% [95% CI: 9.9%, 19.9%]); the result was highly significant (p<0.001) (Table 1).

Relative to placebo, guselkumab induction treatment also resulted in significantly greater proportions of participants achieving the major secondary endpoints (highly significant, p<0.001).

Primary Endpoint: Based on the primary analysis of clinical remission at Week I-12, a significantly greater proportion of participants in the 200 mg IV guselkumab group (22.6%) were in clinical remission at Week I-12 compared with placebo (7.9%; adjusted treatment difference: 14.9% [95% CI: 9.9%, 19.9%]; Table 1)

### Major Secondary Endpoints:

Based on both the global and US-specific testing procedures, a significantly greater proportion of participants in the 200 mg IV guselkumab group achieved symptomatic remission (also known as clinical remission) (adjusted treatment difference: 29.5% [95% CI: 22.9%, 36.1%]; Table 10), endoscopic healing (adjusted treatment difference: 16.0% [95% CI: 10.5%, 21.4%]; Table 4), clinical response (adjusted treatment difference: 34.2% [95% CI: 27.3%, 41.1%]; Table 2), histologic-endoscopic musical healing (adjusted treatment difference: 15.9% [95% CI: 10.9%, 20.9%]; Table 8), fatigue response (adjusted treatment difference: 19.8% [95% CI: 13.1%, 26.4%]; Table 9), all at Week I-12, and symptomatic remission at Week I-4 (adjusted treatment difference: 10.3% [95% CI: 4.8%, 15.7%]), compared with the placebo group (Table 10).
Based on the global testing procedure, a significantly greater proportion of participants in the 200 mg IV guselkumab group achieved IBDQ remission at Week I-12 (adjusted treatment difference: 22.1% [95% CI: 15.1%, 29.2%]; Table 7). Note that IBDQ remission at Week I-12 was not considered as a major secondary endpoint in the US-specific testing procedure.
The 200 mg IV guselkumab group was not significantly different from the placebo group for symptomatic remission at Week I-2 (adjusted treatment difference: 3.0% [95% CI: −1.5%, 7.5%]; Table 10).
Although a greater proportion of participants in the 200 mg IV guselkumab group achieved endoscopic normalization at Week I-12 compared with the placebo group (adjusted treatment difference: 10.1% [95% CI: 5.9%, 14.3%]; Table 3), statistical significance could not be claimed for this endpoint because the previous endpoint in the testing hierarchy (symptomatic remission at Week I-2) was not significant.
Separation between the guselkumab and placebo groups for symptomatic remission was observed as early as 4 weeks after the first dose and continued through Week I-12 (Table 10).

### Subgroup Analyses by ADT-Failure Status:

Greater efficacy was observed with guselkumab vs. placebo for the primary and all major secondary endpoints (except for symptomatic remission at Week I-2) for both the ADT nonfailure and the ADT-failure subgroups.
In general, across the treatment groups, the proportions of participants who met the primary and major secondary endpoints were greater in the ADT non-failure subgroup compared to the ADT-failure subgroup.
Except for fatigue response at Week I-12, across the primary and major secondary endpoints, the treatment effect (vs. placebo) was greater in the ADT non-failure subgroup as compared with the ADT-failure subgroup.

Clinical/Symptomatic Response—As early as Week 2 and increasing through Week 12, greater symptomatic improvement was seen in patients treated with GUS compared with PBO (see Table 14). At Weeks 2, 4, 8, and 12, symptomatic response (clinical response) was achieved by GUS- vs PBO-treated pts in 34.0% vs 23.6%, 53.2% vs 30.0%, 66.0% vs 39.6%, and 71.7% vs 35.0% (all p<0.01), respectively. Percentages of patients who achieved symptomatic remission at Week 2 with GUS vs PBO were 12.1% vs 9.3% (p=0.210); and at Weeks 4, 8, and 12 were 22.6% vs 12.9%, 39.7% vs 20.7%, and 49.9% vs 20.7% (all p<0.001), respectively. As shown in Table 15, mean changes from baseline in absolute number of stools per day at Weeks 2, 4, 8 and 12 were −1.19 vs −0.61, −2.05 vs −0.75, −2.75 vs −1.31, and −3.15 vs −1.36 (all p<0.01). Percentages of patients with stool frequency subscore of 0 or 1 at Weeks 2, 4, 8, and 12 were 26.1% vs 18.2%, 41.3% vs 25.4%, 53.4% vs 29.6%, and 60.1% vs 31.8% (all p<0.05), respectively. Mean changes from baseline in rectal bleeding subscores at Weeks 2, 4, 8, and 12 were −0.6 vs −0.5, −0.8 vs −0.5, −1.1 vs −0.6, and −1.2 vs −0.6 (all p<0.01), respectively. Percentages of patients with rectal bleeding subscores of 0 at Week 2 were 24.2% vs 19.3% (p=0.110); at Weeks 4, 8, and 12 were 36.8% vs 22.9%, 55.8% vs 33.2%, and 64.6% vs 28.6% (all p<0.001), respectively. Treatment differences were achieved at Week 12 for all endpoints evaluated (Table 14).

Safety: Table 13 provides an overall summary of adverse events through Week I-12 for the Safety Analysis set (n=701). Overall, Guselkumab 200 mg IV was safe and well tolerated by participants during the 12-week treatment period. No new safety concerns were identified based on adverse events and laboratory investigations. Adverse events are discussed below; laboratory result observations were consistent with QUASAR Induction Study 1. Similar results were observed for the Safety All Treated Analysis Set.

### Safety Data Through Week I-12 Based on the Safety Analysis Set:

The average duration of follow-up was similar across treatment groups.
The proportion of participants reporting 1 or more adverse events (AEs) in the 200 mg IV guselkumab group was similar to the placebo group.
The system organ classes (SOCs) that were most frequently reported were Infections and Infestations (15.7% guselkumab; 15.0% placebo) and Gastrointestinal Disorders (10.7% guselkumab; 16.4% placebo).
The most common preferred terms (PTs) in the 200 mg IV guselkumab group were COVID-19 (5.0% guselkumab; 4.3% placebo), Anaemia (4.8% guselkumab; 6.8% placebo), and Headache (2.9% guselkumab; 2.9% placebo).
Seven participants had AEs reported within 1 hour of infusion (6 [1.4%] in the 200 mg IV guselkumab group, and 1 [0.4%] in the placebo group); none of these AEs were serious or resulted in the discontinuation of study intervention. There were no anaphylactic reactions or serum sickness reactions reported.
Three deaths were reported (1 in the 200 mg IV guselkumab group, and 2 in the placebo group).
The proportion of participants reporting 1 or more serious AEs was numerically lower in the 200 mg IV guselkumab group (2.9%) compared with the placebo group (7.5%). The majority of SAEs were exacerbations of UC (PT Colitis ulcerative: 1.4% in the 200 mg IV guselkumab group, and 5.0% in the placebo group).
AEs leading to discontinuation of treatment were low across treatment groups (6 [1.4%] in the 200 mg IV guselkumab group and 12 [4.3%] in the placebo group).
The proportions of participants reporting AEs of infections were comparable among treatment groups (66 [15.7%] in the 200 mg IV guselkumab group, and 42 [15.0%] in the placebo group).
Four serious infections were reported (3 [0.7%] in the 200 mg IV guselkumab group, and 1 [0.4%] in the placebo group).
No cases of active tuberculosis (TB) were reported.
One opportunistic infection (PT cytomegalovirus infection) was reported in the placebo group.
Two participants in the 200 mg IV guselkumab group had treatment-emergent malignancies of non-melanomatous skin cancers reported on study days 23 and 32, respectively.
Hepatic laboratory values were similar across treatment groups through Week I-12. Transaminase elevations were low grade (Common Terminology Criteria for Adverse Events [CTCAE] Grade 1). No cases met Hy's law biochemical criteria (ie, total bilirubin ≥2×the upper limit of normal [ULN] and either aspartate aminotransferase [AST] or alanine aminotransferase [ALT]≥3×ULN at the same timepoint)

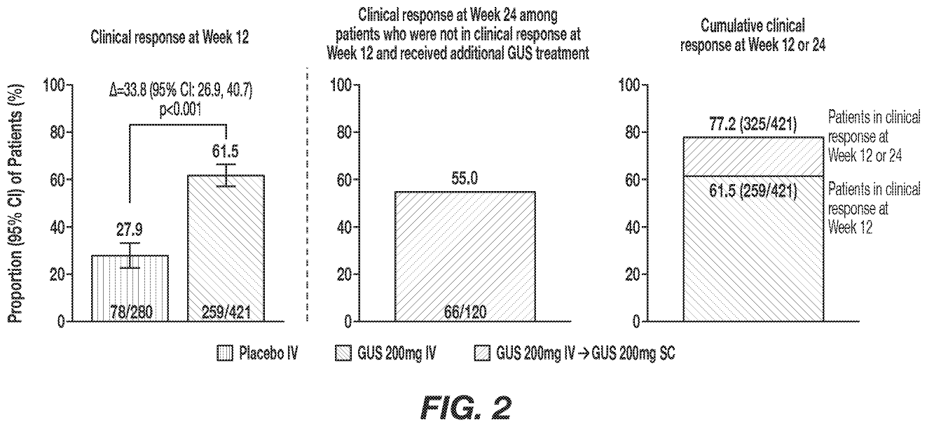

Week 24 Efficacy and Week 32 Safety Data—At Week 12, clinical response was achieved by a higher percentage of GUS-treated patients compared with PBO-treated patients. As shown in FIG. 2, of the GUS-treated patients who were not in clinical response at Week 12, 55% (66/120) achieved clinical response at Week 24. Clinical response at Week 12 or 24 was achieved by 77.2% of patients randomized to GUS 200 mg IV at baseline. Both patients with or without prior ADT-failure benefitted from continued treatment with GUS 200 mg SC through Week 24. For patients who received PBO IV→GUS 200 mg IV, clinical response at Week 24 (69.7%) was similar to Week 12 clinical response following GUS 200 mg IV induction (61.5%). The analysis in FIG. 2 includes only patients with modified Mayo score 5-9 at induction baseline. Clinical response is defined as a decrease from baseline in the modified Mayo score by ≥30% and ≥2 points, with either a ≥1-point decrease from baseline in the rectal bleeding subscore or a rectal bleeding subscore of 0 or 1. Patients who had a prohibited change in UC medication, an ostomy or colectomy, or discontinued study agent due to lack of efficacy or an AE of worsening of UC or other reasons except for COVID-19 related reasons (excluding COVID-19 infection) or regional crisis in Russia and Ukraine prior to the designated timepoint were considered not to have achieved clinical response at the designated timepoint. Patients who were missing one or more Mayo subscore pertaining to clinical response at the designated timepoint were considered not to have achieved clinical response. The adjusted treatment difference and confidence intervals were based on the Wald statistic with Cochran-Mantel-Haenszel weight. The p-values were based on the Cochran-Mantel-Haenszel chi-square test, stratified by ADT-failure status (Yes/No) and concomitant use of corticosteroids at baseline (Yes/No).

As shown in Table 16, safety findings were consistent with the Week 12 results; no new safety concerns were noted. The most frequent adverse events among all GUS-treated patients (n=586) were COVID-19 (7.2%), worsening UC (4.6%), anemia (5.1%).

More than 3/4 of patients with moderately to severely active UC treated with GUS 200 mg IV induction achieved clinical response at Week 12 or 24. No new safety concerns for GUS were identified through Week 32 (Death was reported in 2 placebo-treated patients and 1 GUS-treated patient, all were cardiovascular in nature. The patient in GUS arm had substantial cardiovascular risk factors.). No AEs within 1 hour of infusion were considered serious or resulted in treatment discontinuation. The incidence of anti-drug antibodies (ADA) through Week 12 was low (1.5%, 6/409), and none of the ADA-positive subjects had neutralizing antibodies.

Effect on Histology—Colonic biopsies were collected during endoscopy at baseline and Week 12 to evaluate treatment effect on histology measured by using three methods: Geboes, Robarts Histopathology Index (RHI), and Nancy Histological Index (NHI). Histologic improvement, histologic remission, histo-endoscopic mucosal improvement (HEMI), and the combination of histologic remission and endoscopic normalization (deep HEMI) were evaluated at Week 12.

Histologic activity at baseline was similar across the GUS and PBO cohorts: mean Geboes total score (11.8 vs 11.9, respectively), mean RHI (16.6 vs 16.6), and mean NHI (2.7 vs 2.8). The proportion of patients who achieved histologic improvement at Week 12 was 44.9% among GUS-treated pts and 21.4% among those treated with PBO (adjusted A: 23.7%; nominal p<0.001; Table 17). Histologic remission at Week 12 (as defined by Geboes) was achieved by 39.9% and 18.6% of patients receiving GUS and PBO, respectively (adjusted A: 21.5%; nominal p<0.001). Identical results were seen across two additional definitions of histologic remission (i.e., RHI and NHI; Table 17). The proportion of GUS-treated pts who achieved HEMI at Week 12 was greater than PBO-treated pts and highly significant (23.5% vs 7.5%, respectively; adjusted A: 16.2%; p<0.001). Lastly, deep HEMI at Week 12 was achieved by a greater proportion in the GUS group than PBO group (13.5% vs 3.9%, respectively; adjusted A: 9.8%; nominal p<0.001). Patients experienced clinically meaningful improvements in histologic and histo-endoscopic outcomes at Week 12.

Biomarkers—C-reactive protein (CRP) and fecal calprotectin (FeCal) are non-invasive inflammatory biomarkers used to assess disease activity in UC. The effect of GUS treatment on CRP and FeCal through Week 12 in patients with elevated CRP and/or FeCal is shown in Table 18 and described below. CRP and FeCal were assessed at baseline, Week 4, Week 8 (for CRP only), and Week 12. A total of 701 patients were evaluated in this study and approximately 50% of randomized patients had a history of inadequate response/intolerance to previous advanced therapies for UC; 47.4% of these patients had inadequate response/intolerance to two or more advanced therapy classes. Median baseline concentrations of CRP and FeCal were similar between the GUS-treated and PBO-treated cohorts (4.34 vs 3.83 mg/L and 1651.00 vs 1606.00 mg/kg, respectively).

Among patients with elevated CRP and/or FeCal at baseline, greater reductions in CRP and FeCal were observed at the earliest timepoint assessed (Week 4) with GUS and continued through Week 12 compared with PBO (Table 18). At baseline, 248 GUS-treated patients and 160 PBO-treated patients had elevated CRP (>3 mg/L); the proportions were similar between treatment groups (58.9% vs 57.1%, respectively). Among these patients, median change from baseline in CRP concentration (mg/L) at Week 12 for the GUS and PBO cohorts were −3.99 and −0.51 mg/L, respectively (nominal p<0.001); the proportions of patients achieving 50% and 75% reduction in CRP levels (or ≤3 mg/L) at Week 12 were higher for GUS-treated patients than those receiving PBO (59.7% vs 28.1% and 47.2% vs 19.4%, respectively; both nominal p<0.001). Similarly, a higher proportion of GUS-treated patients achieved CRP<3 mg/L at Week 12 than PBO-treated patients (40.3% vs 16.3%, nominal p<0.001) and 39.1% of GUS-treated patients versus 25.0% of PBO achieved CRP≤3 mg/L at Week 8; 34.3% of GUS-treated patients versus 21.9% of PBO achieved CRP≤3 mg/L at Week 4.

At baseline, 333 GUS-treated patients and 225 PBO-treated patients had elevated FeCal (>250 mg/kg); the proportions were similar between treatment groups (79.1% vs 80.4%, respectively). Median change from baseline in FeCal concentration (mg/kg) for patients with elevated FeCal at baseline at Week 12 were −800.00 and −86.00 mg/kg for the GUS and PBO cohorts, respectively (nominal p<0.001); the proportions of patients achieving 50% and 75% reduction in FeCal levels (or ≤250 mg/kg) at Week 12 were higher for GUS-treated patients than those receiving PBO (51.1% vs 33.8% and 41.4% vs 23.6%, respectively; both nominal p<0.001). A larger percentage of patients treated with GUS achieved FeCal≤250 mg/kg at Week 12 than patients treated with PBO (29.4% vs 17.3%, nominal p<0.001) and 14.1% of GUS-treated patients versus 8.9% of PBO achieved FeCal≤250 mg/kg at Week 4.

Patients with moderately to severely active UC and elevated inflammatory markers treated with GUS 200 mg IV induction showed greater improvement from baseline in both CRP and FeCal levels compared with PBO. Differences were observed as early as the first assessments at Week 4 and continued through Week 12.

### Phase 3 Weeks 1-12 Results and Analysis

Early onset of symptomatic improvement was achieved, as shown in Tables 19-25 below. Primary analysis population included all randomized patients with a modified Mayo score of 5 to 9 who received at least 1 (partial or complete) dose of study intervention. Symptomatic remission at Weeks 2, 4, and 12 were major secondary endpoints that were controlled for Type 1 error. The p-value for all other analyses was nominal. Patients who had a prohibited change in UC medication, an ostomy or colectomy, or discontinued study agent due to lack of efficacy or an adverse event of worsening of UC, or due to other reasons except for COVID-19 related reasons (excluding COVID-19 infection) or regional crisis in Russia and Ukraine prior to Week 12 were considered not to have achieved a specified binary endpoint and had baseline observation carried forward for a specified continuous endpoint. Patients who were missing one or more components pertaining to a specified binary endpoint were considered not to have achieved the endpoint.

Guselkumab 200 mg IV induction was effective in improving symptoms as early as one week after the first dose in patients with moderately to severely active UC. Table 19 demonstrates symptomatic remission achieved as early as one week after the first dose. Table 20 demonstrates symptomatic response achieved as early as one week after the first dose. Table 21 demonstrates achievement of stool frequency subscore of 0 or 1 as early as one week after the first dose. Table 22 demonstrates achievement of rectal bleeding subscore of 0 as early as one week after the first dose. Table 23 demonstrates change in absolute stool number as early as one week after the first dose. Table 24 demonstrates change in stool frequency and rectal bleeding subscores as early as one week after the first dose. Table 25 demonstrates achievement of symptomatic Mayo Score improvement as early as two weeks after the first dose. In addition, symptomatic improvements continued to increase through Week 12 with guselkumab treatment.

Phase 3 Maintenance Study Trial Design: The Phase 3 Maintenance Study is a randomized withdrawal, double-blind, placebo-controlled, parallel-group, multicenter study. The target population consists of participants with moderately to severely active UC with a history of an inadequate response or failure to tolerate conventional (i.e., 6-MP, AZA, or corticosteroids) or advanced therapy (i.e., TNFα antagonists, vedolizumab, or tofacitinib) who were in clinical response 12 weeks after receiving guselkumab IV induction therapy. At Week I-0, participants must have moderately to severely active UC, defined as a modified Mayo score of 5 to 9, inclusive, Mayo rectal bleeding subscore ≥1 and a Mayo endoscopy subscore ≥2, using the Mayo endoscopy subscore obtained during the central review of the video endoscopy. The QUASAR program also allows for the enrollment of participants with a modified Mayo score of 4, which is capped at ≤5% of the total population enrolled in the program. The protocol was amended per health authority feedback so that the target population should be based on only participants with a modified Mayo score of 5 to 9.

Treatment allocation: Participants who were in clinical response 12 weeks after receiving guselkumab IV induction treatment (i.e., guselkumab clinical responders at Week I-12 and placebo crossover guselkumab responders at Week I-24 from Induction Study 1 or Induction Study 2 were randomized at Week M (Maintenance)-0 in a 1:1:1 ratio to 1 of 3 treatment groups: (i) Placebo SC every 4 weeks (q4w) (ii) Guselkumab 100 mg SC every 8 weeks (q8w) and (iii) Guselkumab 200 mg SC q4w.

Participants were allocated to an intervention group using permuted block randomization with clinical remission status at maintenance baseline (Yes/No), concomitant use of corticosteroids at maintenance baseline (Yes/No), and induction treatment (guselkumab 400 mg IV, guselkumab 200 mg IV, and placebo IV→guselkumab 200 mg IV) as stratification variables.

Randomized participants who lost clinical response (i.e., not meeting the criteria for clinical response) were eligible for a single blinded dose adjustment, beginning at a visit between Week M-8 and Week M-32, to receive guselkumab 200 mg SC q4w. In addition to the aforementioned randomized population, guselkumab induction 24-Week responders and induction placebo responders at Week I-12 from Induction Study 1 or Induction Study 2 entered the Maintenance Study but were not randomized and are not included in the key efficacy analyses herein.

Treatment duration/Trial duration: Duration of treatment was 40 weeks and trial duration was 44 weeks for this reporting period. This report provides the results for the primary and major secondary endpoints based on the global and US-specific multiple testing procedures.

Primary endpoint: The primary endpoint was clinical remission at Week M-44, defined as a Mayo stool frequency subscore of 0 or 1 and not increased from induction baseline, a Mayo rectal bleeding subscore of 0, and a Mayo endoscopy subscore of 0 or 1 with no friability present on the endoscopy.

### Major Secondary Endpoints (Ordered by the Global Testing Procedure):

Symptomatic remission at Week M-44: a Mayo stool frequency subscore of 0 or 1 and not increased from induction baseline, and a Mayo rectal bleeding subscore of 0.
Endoscopic healing at Week M-44: a Mayo endoscopy subscore of 0 or 1 with no friability present on the endoscopy. Corticosteroid-free (i.e., not requiring any treatment with corticosteroids for at least 8 weeks prior) clinical remission at Week M-44.
Maintenance of clinical response at M-44: clinical response at Week M-44 among subjects achieving clinical response at maintenance baseline. Clinical response is defined as a decrease from induction baseline in the modified Mayo score by ≥30% and ≥2 points, with either a ≥1-point decrease from baseline in the rectal bleeding subscore or a rectal bleeding subscore of 0 or 1.
Histologic-endoscopic mucosal healing at Week M-44: achieving a combination of histologic healing and endoscopic healing, where histologic healing is defined as neutrophil infiltration in <5% of crypts, no crypt destruction, and no erosions, ulcerations or granulation tissue according to the Geboes grading system (i.e., Geboes score ≤3.1).
IBDQ remission at Week M-44: total IBDQ score ≥170.
Fatigue response at Week M-44: ≥7-point improvement from induction baseline in PROMIS-Fatigue Short Form 7a.
Maintenance of clinical remission at M-44: clinical remission at Week M-44 among the participants achieving clinical remission at maintenance baseline.
Endoscopic normalization at Week M-44: a Mayo endoscopy subscore of 0.

Expected effect size and planned sample size: Assuming a 25% clinical remission rate at Week M-44 for placebo and 45% for each of the guselkumab treatment groups (assumed rates were based on data from the ustekinumab UC maintenance study [CNTO1275UCO3001]), 118 participants in each randomized group (354 participants in total) provided a statistical power of 90% at a significance level of 0.05 (2 sided) for the primary endpoint. However, the targeted number in the primary analysis population was increased to 484 participants in order to have at least 90% power for the majority of the major secondary endpoints (note that the endpoints of maintenance of clinical remission at Week M-44 and endoscopic normalization at Week M-44 were not powered at 90%).

### Statistical Considerations:

Analysis set for efficacy—The Randomized Full Analysis Set includes all participants with a modified Mayo score of 5 to 9 at Week I-0 who were randomized and received at least 1 (partial or complete) dose of study intervention in this maintenance study. Participants were analyzed according to their randomized study intervention regardless of the study intervention they actually received.

Analysis set for safety—The Randomized Safety Analysis Set includes all participants with a modified Mayo score of 5 to 9 at Week I-0 who were randomized and received at least 1 (partial or complete) dose of study intervention in this maintenance study. There are 2 additional safety sets. The first is the Safety Analysis Set, which includes both randomized and nonrandomized participants with a modified Mayo score of 5 to 9 at Week I-0 who were treated in this maintenance study, and the second is the All Treated Analysis Set, which includes all treated participants in this maintenance study with a modified Mayo score of 4 to 9 at Week I-0. In general, participants were analyzed according to their assigned treatment. However, participants assigned to placebo who incorrectly received guselkumab at any time were analyzed in the guselkumab group from the time of first guselkumab administration onward; participants assigned to guselkumab who received only placebo were analyzed in the placebo group.

Intercurrent events (ICEs) and corresponding strategies were applied to the analysis of the efficacy endpoints. Participants who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (ICE 4) prior to the analysis timepoint were considered not to have achieved the binary endpoints (i.e., composite strategy). For participants who discontinued study agent due to COVID-19 related reasons (excluding COVID-19 infection) or the regional crisis in Russia and Ukraine (ICE 5) prior to the analysis timepoint, their observed values (if available) were used (i.e., treatment policy). Participants who discontinued study agent due to reasons other than those in ICEs 4 and 5 (ICE 6; composite strategy) prior to the analysis timepoint were considered not to have achieved the binary endpoints.

Comparisons were based on each guselkumab group versus the placebo group. For the primary and major secondary endpoints (except for maintenance of clinical remission), the p-values were based on a Cochran-Mantel-Haenszel (CMH) test (2-sided) stratified by clinical remission status at maintenance baseline (Yes/No) and induction treatment (guselkumab 400 mg IV, guselkumab 200 mg IV, placebo IV→guselkumab 200 mg IV). For maintenance of clinical remission, a CMH test (2-sided) stratified by induction treatment was used. The 95% confidence intervals were based on the Wald statistic with Cochran-Mantel-Haenszel weight. For these endpoints, participants who had missing data at the analysis timepoint (after accounting for the intercurrent event strategies) were considered non-responders at that timepoint.

Global testing procedure (countries outside the United States): A hierarchical testing procedure was used to control the overall Type 1 error rate over the primary and major secondary efficacy analyses at the 2-sided 0.05 significance level within a guselkumab dose group. The primary endpoint was tested using a fixed sequence testing procedure, beginning with the high maintenance treatment group (200 mg SC q4w). A major secondary endpoint for a dose group was considered significant only if the previous endpoints for that dose group in the hierarchy and the current endpoint tested positive at the 2-sided 0.05 level of significance. If an endpoint was not significant, all subsequent tests in the hierarchy for that dose were considered not to be significant.

US-specific testing procedure: A hierarchical testing procedure was used to strongly control the overall Type 1 error rate at the 0.05 level across the primary and major secondary endpoints and across the 2 guselkumab treatment groups. An endpoint would be considered significant only if all the previous endpoints in the hierarchy and the current endpoint tested positive at the 2-sided 0.05 level of significance. If an endpoint was not significant, all subsequent tests in the hierarchy were considered not to be significant. Note that the ranking order of the endpoints is different for the two testing strategies due to regional preferences. In addition, IBDQ remission at Week M-44 was not included in the US-specific testing procedure.

Primary Objective(s): The primary objective is to evaluate the clinical efficacy and safety of guselkumab as maintenance therapy in participants with moderately to severely active UC who were induced into clinical response with guselkumab.

Executive Summary: Based on the pre-specified global and US-specific multiple testing procedures, Guselkumab maintenance treatments at both 200 mg SC q4w and 100 mg SC q8w resulted in significantly greater proportions of participants in clinical remission at Week M-44 (primary endpoint; Table 26). Adjusted treatment differences of ≥25 percentage points were observed, and the results were highly statistically significant (i.e., p<0.001). Relative to placebo, guselkumab maintenance treatment (at both doses evaluated) also resulted in significantly greater proportions of participants who achieved each of the major secondary endpoints (Table 27). Subcutaneous maintenance regimens of guselkumab 200 mg SC q4w or guselkumab 100 mg SC q8w through Week M-44 were well tolerated, and safety data are consistent with the known safety profile of guselkumab. The benefit risk profile is favorable for GUS in the treatment of UC.

Topline Results Summary: A total of 846 participants entered the Maintenance Study and were treated across 254 sites in 32 countries: 599 were randomized and 247 were nonrandomized. Among 599 randomized and treated participants, 568 (94.8%) participants had a modified Mayo score of 5 to 9 at Week I-0 (target population to be used for the efficacy and safety analyses below). Any impact to the study due to COVID-19 or the regional crisis was negligible.

Disposition and baseline characteristics for the Randomized Full Analysis Set (n=568): A total of 12.0% of participants discontinued study intervention prior to Week M-44, and the proportions across treatment groups were comparable: 11.6% in the guselkumab 200 mg SC q4w group, 10.6% in guselkumab 100 mg SC q8w group, and 13.7% in the placebo group. The most common reasons for study intervention discontinuation prior to Week M-44 were adverse events due to worsening UC (3.3%) and withdrawal by subject (3.3%). The demographics, disease characteristics, UC medication history, and concomitant UC medications were generally well-balanced across the treatment groups.

At induction baseline, the majority of participants were white (73.2%), and 54.8% of the participants were male. The mean age was 40.7 years (range 18 to 79 years). The mean duration of UC disease was 7.81 years, the median modified Mayo score was 7.0 (mean=6.9), 63.9% had severe disease as defined by a modified Mayo score 7 to 9, 66.4% of participants had an endoscopy subscore of 3 (i.e., severe disease), 45.2% of participants had extensive disease, the median fecal calprotectin was 1605.0 mg/kg, and the median C-reactive protein (CRP) concentration was 3.9 mg/L (Table 29).

At maintenance baseline, the median modified Mayo score was 2.0 (mean=2.5), the median fecal calprotectin was 303.5 mg/kg, and the median C-reactive protein (CRP) concentration was 1.5 mg/L. At maintenance baseline, 34.2% of participants were in clinical remission, and 39.1% had endoscopic healing (Table 30).

At induction baseline, a total of 240 (42.3%) participants had a history of advanced therapy (ADT) failure (102 [42.5%] of those participants had a history of failing two or more ADT classes); 328 (57.7%) had failed conventional therapy but not advanced therapy (“ADT non-failure”) and the majority 309 (94.2%) of these participants were ADT-naïve. A total of 40.0% of participants were receiving corticosteroids at induction baseline and 22.2% were receiving immunomodulators (6-mercaptopurine, azathioprine, or methotrexate).

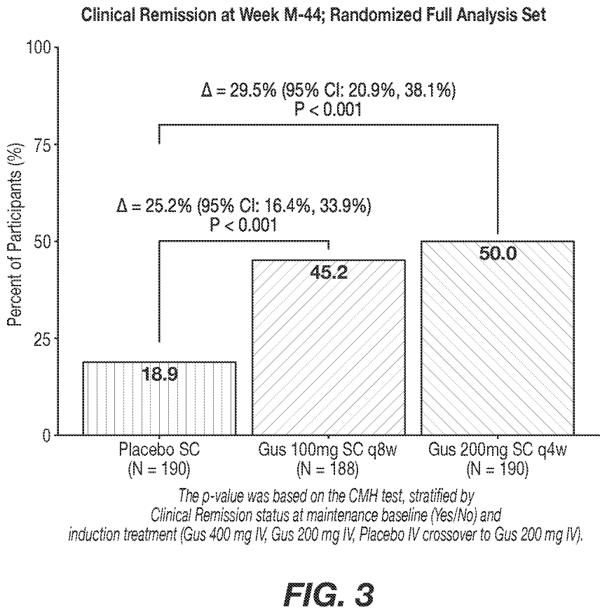

Primary Endpoint: The study is considered to be a positive study. Significantly greater proportions of participants in the guselkumab 200 mg SC q4w group (50.0%) and guselkumab 100 mg SC q8w group (45.2%) were in clinical remission at Week M-44 compared with the placebo group (18.9%; adjusted treatment difference 29.5% [95% CI: 20.9%, 38.1%] and 25.2% [95% CI: 16.4%, 33.9%] respectively) (FIG. 3; Table 26).

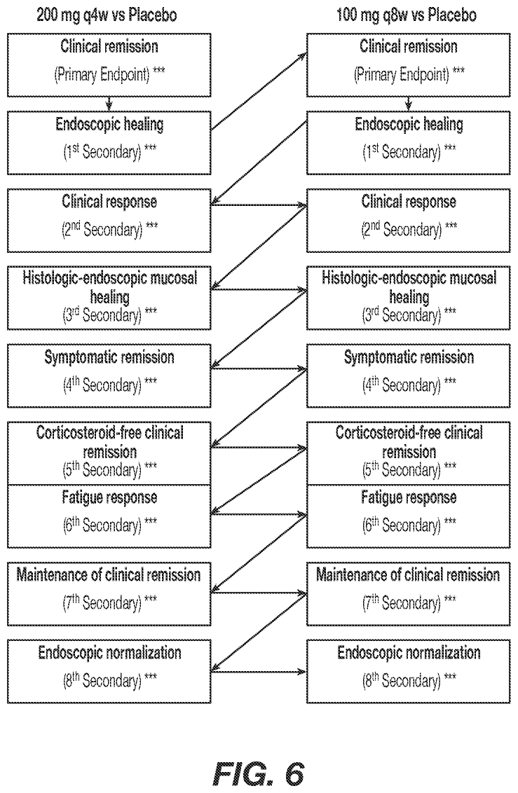

Major Secondary Endpoints: Based on both the global (Table 27) and US-specific testing procedures (FIG. 6), compared with the placebo group, a significantly greater proportion of participants in the guselkumab 200 mg SC q4w group and guselkumab 100 mg SC q8w group achieved each of the major secondary endpoints at Week M-44 (Table 28). For FIG. 6, asterisks denote the level of significance achieved (* p<0.05; ** p<0.01; *** p<0.001. The p-values were based on the Cochran-Mantel-Haenszel test.).

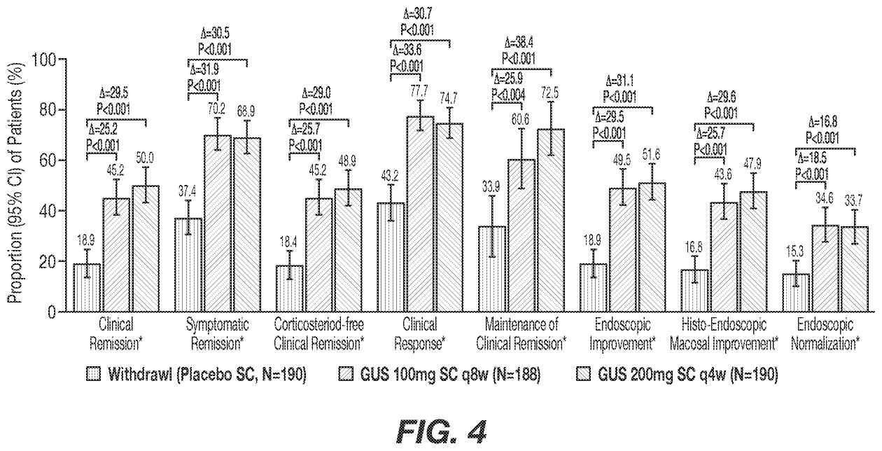

A significantly higher proportion of patients treated with guselkumab subcutaneous maintenance therapy achieved the primary and major secondary endpoints evaluated at Week 44 compared with withdrawal (placebo) patients (FIG. 4). Major secondary endpoints evaluated at Week 44 included symptomatic remission, corticosteroid-free clinical remission, clinical response, maintenance of clinical remission, endoscopic improvement, histo-endoscopic mucosal improvement, and endoscopic normalization.

For FIG. 4: Adjusted treatment difference (Δ) was based on the Wald statistic with Cochran-Mantel-Haenszel (CMH) weight. P-values were based on the CMH test, stratified by clinical remission status at maintenance baseline and induction treatment. ^{a} Clinical remission: A Mayo stool frequency subscore of 0 or 1 and not increased from baseline, a Mayo rectal bleeding subscore of 0, and a Mayo endoscopy subscore of 0 or 1 with no friability. ^{b} Symptomatic remission: A stool frequency subscore of 0 or 1 and not increased from baseline, and a rectal bleeding subscore of 0. ^{c} Corticosteroid-free clinical remission: Not requiring any treatment with corticosteroids for at least 8 weeks prior to Week 44 and also meeting the criteria for clinical remission. ^{d} Clinical response: A decrease from baseline in the modified Mayo score by ≥30% and ≥2 points, with either a ≥1-point decrease from baseline in the rectal bleeding subscore or a rectal bleeding subscore of 0 or 1. ^{e} Maintenance of clinical remission: Clinical remission at Week 44 among patients in clinical remission at maintenance baseline. ^{f} Endoscopic improvement: An endoscopy subscore of 0 or 1 with no friability present on the endoscopy. ^{g} Histo-endoscopic mucosal improvement: Achieving a combination of histologic improvement (neutrophil infiltration in <5% of crypts, no crypt destruction, and no erosions, ulcerations or granulation tissue per Geboes grading system) and endoscopic improvement. ^{h} Endoscopic normalization: An endoscopy subscore of 0.

Subgroup Analyses by ADT failure status: Greater efficacy was observed in both guselkumab groups vs. placebo for the primary and all major secondary endpoints for both the ADT non-failure and the ADT failure subpopulations (Tables 31 and 32). In general, across the treatment groups, the proportions of participants who met the primary and major secondary endpoints were greater in the ADT non-failure subpopulation compared to the ADT failure subpopulation. Across the primary and major secondary endpoints, the treatment effects (vs. placebo) for both guselkumab groups were generally higher, and in some cases, comparable, in the ADT failure subpopulation as compared with the ADT non-failure subpopulation.

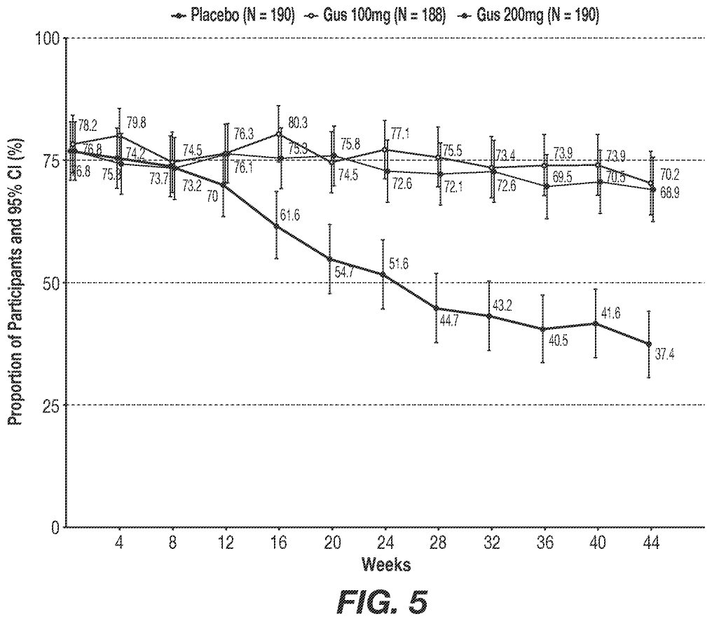

Other Endpoints A greater proportion of participants in the guselkumab 200 mg SC q4w group and guselkumab 100 mg SC q8w group achieved histologic healing at Week M-44 compared with the placebo group (adjusted treatment difference in guselkumab 200 mg SC q4w group: 32.6% [95% CI: 23.3%, 41.9%], guselkumab 100 mg SC q8w group: 33.6% [95% CI: 24.3%, 42.9%]) (Table 33). Over time, the proportion of participants in symptomatic remission was sustained through Week M-44 in the guselkumab groups while the proportion of participants in symptomatic remission decreased in the placebo group. Separation from the placebo group was observed at Week M-16 and continued to increase through Week M-44. (FIG. 5).

Safety: Subcutaneous maintenance regimens of guselkumab 200 mg SC q4w or guselkumab 100 mg SC q8w through Week M-44 were well tolerated, and safety data are consistent with the known safety profile of guselkumab. Table 34 provides an overall summary of adverse events (AEs) through Week M-44 (up to dose adjustment) for the Randomized Safety Analysis Set. No new safety concerns were identified based on adverse events and laboratory investigations. The overall AE rates were similar across the treatment groups. The rate of serious AEs (SAEs) was numerically higher in the guselkumab 200 mg SC q4w group compared to the placebo group in the Randomized Safety Analysis Set, but this trend was less apparent in the Safety Analysis Set and the All Treated Analysis Set.

Safety Data through Week M-44 Based on Randomized Safety Analysis Set: The average duration of follow-up in the placebo group (34.0 weeks) was shorter than that in the guselkumab 200 mg SC q4w group (39.2 weeks) and the guselkumab 100 mg SC q8w group (40.5 weeks). (Table 34). The proportions of participants reporting 1 or more AEs in the guselkumab 200 mg SC q4w group and the guselkumab 100 mg SC q8w group (70.0% and 64.5%) were comparable to the placebo group (68.2%) (Table 34). The system organ classes (SOCs) with the most frequently reported AEs were Infections and Infestations (31.1% guselkumab 200 mg SC q4w; 31.7% guselkumab 100 mg SC q8w; 32.8% placebo), and Gastrointestinal Disorders (25.8% guselkumab 200 mg SC q4w; 24.7% guselkumab 100 mg SC q8w; 37.0% Placebo). The most common preferred terms (PTs) in the combined guselkumab group were COVID-19 (11.2%), Colitis ulcerative (11.2%), and Arthralgia (6.1%); the PTs in the placebo group were 14.1%, 29.7%, and 6.8%, respectively.

The proportion of participants reporting 1 or more SAEs was 6.3%, 2.7% and 0.5% in the guselkumab 200 mg SC q4w, guselkumab 100 mg SC q8w, and placebo groups, respectively (Table 34). There was no clear pattern in the SAEs with respect to individual PTs or SOCs, and the majority of the SAEs were assessed as unrelated by the investigator. The differences in the SAE rates between the guselkumab 200 mg SC q4w group and the placebo group were less apparent in the Safety Analysis Set and the All Treated Analysis Set which included a larger number of participants, including nonrandomized guselkumab induction 24-Week responders and induction placebo responders. The proportion of participants with AEs leading to discontinuation of study agent was numerically higher in the placebo group than in the guselkumab 100 mg SC q4w and guselkumab 200 mg SC q8w groups with 6.8%, 3.8%, and 2.6%, respectively (Table 34). The proportion of participants reporting 1 or more AEs of severe intensity was similar among treatment groups (Table 34). The proportions of participants reporting AEs of infections were comparable among treatment groups (Table 34). Serious infections were infrequent (2 [1.1%] in the guselkumab 200 mg SC q4w group [1 participant with moderate bacterial infection at the venipuncture site and 1 participant with gangrenous appendicitis], and 1 [0.5%] in the guselkumab 100 mg SC q8w group [intergluteal abscess]). There were no notable differences in the proportions of participants with post-baseline maximum toxicity Grade ≥1 chemistry and hematology laboratory results across the placebo and the guselkumab groups. Grade 3 and Grade 4 chemistry and hematology laboratory values were infrequent. No cases met Hy's law biochemical criteria (i.e., total bilirubin ≥2×the upper limit of normal [ULN] and either aspartate aminotransferase [AST] or alanine transferase [ALT]≥3×ULN at the same timepoint).

AEs of Special Interest and other AEs in the Maintenance Study through Week M-44, based on the All Treated Analysis Set: No deaths were reported. There were no cases of active tuberculosis reported. There were no cases of opportunistic infection reported based on the opportunistic infection Standardised MedDRA Queries (SMQ). Eight participants reported 9 malignancies; Three participants had 4 malignancies of non-melanoma skin cancer (all in the randomized placebo group). Three participants had breast cancer (2 randomized placebo group and 1 nonrandomized placebo group). One participant had clear cell renal cell carcinoma (nonrandomized guselkumab 200 mg SC q4w group), and one participant had adenocarcinoma of the rectum (nonrandomized guselkumab 200 mg SC q4w group). There was 1 participant in the randomized guselkumab 200 mg SC q4w group with a reported Hemorrhagic Stroke which was considered a Major Adverse Cardiovascular Event (MACE). There were no confirmed Hy's law cases, no cases of Treatment-emergent Serious Hepatic Disorder Adverse Events, and no Treatment-emergent Hepatic Disorder Adverse Events Leading to Discontinuation of Study Agent. There were 2 participants with reported venous thromboembolic AEs. One case was in the randomized guselkumab 200 mg SC q4w group, and the other case was in the nonrandomized guselkumab 200 mg SC q4w group. Both participants had a history of VTE. There were no cases of anaphylaxis or serum-sickness reported.

TABLE 1
Primary Endpoint Analysis (Primary Estimand): Number
of Subjects in Clinical Remission at Week I-12; Full
Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects in clinical | 22 (7.9%) | 95 | (22.6%) |
| remission^{a,b,c} | | | |
| 95% CI for treatment | (4.7%, 11.0%) | (18.6%, 26.6%) | |
| proportion^{d} | | | |
| Adjusted treatment | | 14.9% | (9.9%, 19.9%) |
| difference (95% CI)^{e} | | | |

| | |
|---|---|
| p-value^{f} | <0.001 |

Note:
Includes only subjects with modified Mayo score 5-9 at induction baseline.
^{a}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to be in clinical remission at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to be in clinical remission at Week I-12.
^{c}Nonresponder Imputation for Missing Data: After accounting for ICEs, subjects who were missing one or more Mayo subscore pertaining to this endpoint (stool frequency, rectal bleeding, or endoscopy) at Week I-12 were considered not to be in clinical remission.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 2
Major Secondary Endpoint Analysis (Main Estimand): Number
of Subjects in Clinical Response at Week I-12; Full
Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects in clinical | 77 (27.5%) | 259 | (61.5%) |
| response^{a,b,c} | | | |
| 95% CI for treatment | (22.3%, 32.7%) | (56.9%, 66.2%) | |
| proportion^{d} | | | |
| Adjusted treatment | | 34.2% | (27.3%, 41.1%) |
| difference (95% CI)^{e} | | | |

| | |
|---|---|
| p-value^{f} | <0.001 |

Note:
Includes only subjects with modified Mayo score 5-9 at induction baseline.
^{a}Clinical response is defined as a decrease from induction baseline in the modified Mayo score by ≥30% and ≥2 points, with either a ≥1-point decrease from baseline in the rectal bleeding subscore or a rectal bleeding subscore of 0 or 1.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to be in clinical response at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to be in clinical response at Week I-12.
^{c}Nonresponder Imputation for Missing Data: After accounting for ICEs, subjects who were missing one or more Mayo subscore pertaining to this endpoint (stool frequency, rectal bleeding, or endoscopy) at Week I-12 were considered not to be in clinical response.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 3
Major Secondary Endpoint Analysis (Main Estimand): Number
of Subjects With Endoscopic Normalization at Week I-12;
Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects with endoscopic | 14 (5.0%) | 63 | (15.0%) |
| normalization^{a,b,c} | | | |
| 95% CI for treatment | (2.4%, 7.6%) | (11.6%, 18.4%) | |
| proportion^{d} | | | |
| Adjusted treatment | | 10.1% | (5.9%, 14.3%) |
| difference (95% CI)^{e} | | | |

| | |
|---|---|
| p-value^{f} | <0.001 |

Note:
Includes only subjects with modified Mayo score 5-9 at induction baseline.
^{a}Endoscopic normalization is defined as an endoscopy subscore of 0.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to have achieved endoscopic normalization at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to have achieved endoscopic normalization at Week I-12.
^{c}Nonresponder Imputation for Missing Data: After accounting for ICEs, subjects who were missing the endoscopy subscore at Week I-12 were considered not to have achieved endoscopic normalization.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 4
Major Secondary Endpoint Analysis (Main Estimand): Number
of Subjects With Endoscopic Healing at Week I-12; Full
Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects with endoscopic | 31 (11.1%) | 113 | (26.8%) |
| healing^{a,b,c} | | | |
| 95% CI for treatment | (7.4%, 14.7%) | (22.6%, 31.1%) | |
| proportion^{d} | | | |
| Adjusted treatment | | 16.0% | (10.5%, 21.4%) |
| difference (95% CI)^{e} | | | |

| | |
|---|---|
| p-value^{f} | <0.001 |

Note:
Includes only subjects with modified Mayo score 5-9 at induction baseline.
^{a}Endoscopic healing is defined as an endoscopy subscore of 0 or 1 with no friability present on the endoscopy.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to have achieved endoscopic healing at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to have achieved endoscopic healing at Week I-12.
^{c}Nonresponder Imputation for Missing Data: After accounting for ICEs, subjects who were missing the endoscopy subscore at Week I-12 were considered not to have achieved endoscopic healing.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 5
Number of Subjects With Histologic Healing at Week I-12;
Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects with histologic | 60 (21.4%) | 187 | (44.4%) |
| healing^{a,b,c} | | | |
| 95% CI for treatment | (16.6%, 26.2%) | (39.7%, 49.2%) | |
| proportion^{d} | | | |
| Adjusted treatment | | 23.2% | (16.5%, 29.8%) |
| difference (95% CI)^{e} | | | |

| | |
|---|---|
| p-value^{f} | <0.001 |

Note:
Includes only subjects with modified Mayo score 5-9 at induction baseline.
^{a}Histologic healing is defined as neutrophil infiltration in <5% of crypts, no crypt destruction, and no erosions, ulcerations or granulation tissue according to the Geboes grading system.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to have achieved histologic healing at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to have achieved histologic healing at Week I-12.
^{c}Nonresponder Imputation for Missing Data: After accounting for ICEs, subjects who had an unevaluable biopsy (i.e., a biopsy that was collected, but could not be assessed due to sample preparation or technical errors) or were missing any of the components pertaining to this endpoint (i.e., assessment of neutrophils in epithelium, crypt destruction, or erosions or ulcerations or granulations) at Week I-12 were considered not to have achieved histologic healing.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 6
Number of Subjects in Histologic Remission at Week I-12;
Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects in histologic | 52 (18.6%) | 166 | (39.4%) |
| remission^{a,b,c} | | | |
| 95% CI for treatment | (14.0%, 23.1%) | (34.8%, 44.1%) | |
| proportion^{d} | | | |
| Adjusted treatment | | 21.0% | (14.6%, 27.4%) |
| difference (95% CI)^{e} | | | |

| | |
|---|---|
| p-value^{f} | <0.001 |

Note:
Includes only subjects with modified Mayo score 5-9 at induction baseline.
^{a}Histologic remission is defined as absence of neutrophils from mucosa (both lamina propria and epithelium), no crypt destruction, and no erosions, ulcerations or granulation tissue according to the Geboes grading system. This definition is equivalent to a Robarts Histopathology Index ≤3, with subscores of 0 for lamina propria neutrophils and neutrophils in the epithelium and without ulcers or erosion.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to be in histologic remission at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to be in histologic remission at Week I-12.
^{c}Nonresponder Imputation for Missing Data: After accounting for ICEs, subjects who had an unevaluable biopsy (i.e., a biopsy that was collected, but could not be assessed due to sample preparation or technical errors) or were missing any of the histology components pertaining to this endpoint (i.e., assessment of neutrophils in lamina propria or epithelium, crypt destruction, or erosions or ulcerations or granulations) at Week I-12 were considered not to be in histologic remission.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 7
Major Secondary Endpoint Analysis (Main Estimand): Number
of Subjects in IBDQ Remission at Week I-12; Full Analysis
Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects in IBDQ | 83 (29.6%) | 217 | (51.5%) |
| remission^{a,b,c} | | | |
| 95% CI for treatment | (24.3%, 35.0%) | (46.8%, 56.3%) | |
| proportion^{d} | | | |
| Adjusted treatment | | 22.1% | (15.1%, 29.2%) |
| difference (95% CI)^{e} | | | |

| | |
|---|---|
| p-value^{f} | <0.001 |

Note:
Includes only subjects with modified Mayo score 5-9 at induction baseline.
^{a}IBDQ (Inflammatory Bowel Disease Questionnaire) remission is defined as a total IBDQ score ≥170.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to be in IBDQ remission at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to be in IBDQ remission at Week I-12.
^{c}Nonresponder Imputation for Missing Data: After accounting for ICEs, subjects who were missing the IBDQ total score at Week I-12 were considered not to be in IBDQ remission.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 8
Major Secondary Endpoint Analysis (Main Estimand): Number of
Subjects With Histologic-endoscopic Mucosal Healing at Week
I-12; Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects with histologic- | 21 (7.5%) | 98 | (23.3%) |
| endoscopic mucosal | | | |
| healing^{a,b,c} | | | |
| 95% CI for treatment | (4.4%, 10.6%) | (19.2%, 27.3%) | |
| proportion^{d} | | | |
| Adjusted treatment | | 15.9% | (10.9%, 20.9%) |
| difference (95% CI)^{e} | | | |

| | |
|---|---|
| p-value^{f} | <0.001 |

Note:
Includes only subjects with modified Mayo score 5-9 at induction baseline.
^{a}Histologic-endoscopic mucosal healing is defined as achieving a combination of histologic healing and endoscopic healing. Histologic healing is defined as neutrophil infiltration in <5% of crypts, no crypt destruction, and no erosions, ulcerations or granulation tissue according to the Geboes grading system. Endoscopic healing is defined as an endoscopy subscore of 0 or 1 with no friability present on the endoscopy.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to have achieved histologic-endoscopic mucosal healing at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to have achieved histologic-endoscopic mucosal healing at Week I-12.
^{c}Nonresponder Imputation for Missing Data: After accounting for ICEs, subjects who had an unevaluable biopsy (i.e., a biopsy that was collected, but could not be assessed due to sample preparation or technical errors) or were missing the endoscopy subscore or any of the histology components pertaining to this endpoint (i.e., assessment of neutrophils in epithelium, crypt destruction, or erosions or ulcerations or granulations) at Week I-12 were considered not to have achieved histologic-endoscopic mucosal healing.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 9
Major Secondary Endpoint Analysis (Main Estimand): Number
of Subjects in Fatigue Response at Week I-12; Full Analysis
Set (Study CNTO1959UCO3001; Induction 2)

| | Placebo IV | Guselkumab 200 mg IV | |
|---|---|---|---|
| Analysis set: Full | 280 | 421 | |
| Week I-12 | | | |
| N | 280 | 421 | |
| Subjects in fatigue | 60 (21.4%) | 173 | (41.1%) |
| response^{a,b,c} | | | |
| 95% CI for treatment | (16.6%, 26.2%) | (36.4%, 45.8%) | |
| proportion^{d} | | | |
| Adjusted treatment | | 19.8% | (13.1%, 26.4%) |
| difference (95% CI)^{e} | | | |

| | |
|---|---|
| p-value^{f} | <0.001 |

Note:
Includes only subjects with modified Mayo score 5-9 at induction baseline.
^{a}Fatigue response is defined as ≥7-point improvement from induction baseline in the PROMIS (Patient-Reported Outcomes Measurement Information System) Fatigue Short Form 7a.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to Week I-12 were considered not to be in fatigue response at Week I-12. For subjects who discontinued study agent due to COVID-19 related reasons (excluding COVID-19 infection) or regional crisis (ICE 4) prior to Week I-12, their observed values will be used, if available. Subjects who experienced ICE 5 (discontinued study agent due to reasons other than those in ICEs 3 and 4) prior to Week I-12 were considered not to be in fatigue response at Week I-12.
^{c}Nonresponder Imputation for Missing Data: After accounting for ICEs, subjects who were missing one or more of the PROMIS Fatigue Short Form 7a items at either induction baseline or Week I-12 were considered not to be in fatigue response.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 10
Major Secondary Endpoint Analysis (Main Estimand):
Number of Subjects in Symptomatic Remission at
Week I-2, Week I-4, and Week I-12; Full Analysis
Set (Study CNTO1959UCO3001; Induction 2)

| | Placebo IV | Guselkumab 200 mg IV | |
|---|---|---|---|
| Analysis set: Full | 280 | 421 | |
| Week I-2 | | | |
| N | 280 | 421 | |
| Subjects in symptomatic | 26 (9.3%) | 51 | (12.1%) |
| remission^{a,b,c} | | | |
| 95% CI for treatment | (5.9%, 12.7%) | (9.0%, 15.2%) | |
| proportion^{d} | | | |
| Adjusted treatment | | 3.0% | (−1.5%, 7.5%) |
| difference (95% CI)^{e} | | | |
| p-value^{f} | | 0.210 | |
| Week I-4 | | | |
| N | 280 | 421 | |
| Subjects in symptomatic | 35 (12.5%) | 95 | (22.6%) |
| remission^{a,b,c} | | | |
| 95% CI for treatment | (8.6%, 16.4%) | (18.6%, 26.6%) | |
| proportion^{d} | | | |
| Adjusted treatment | | 10.3% | (4.8%, 15.7%) |
| difference (95% CI)^{e} | | | |
| p-value^{f} | | <0.001 | |
| Week I-12 | | | |
| N | 280 | 421 | |
| Subjects in symptomatic | 57 (20.4%) | 209 | (49.6%) |
| remission^{a,b,c} | | | |
| 95% CI for treatment | (15.6%, 25.1%) | (44.9%, 54.4%) | |
| proportion^{d} | | | |
| Adjusted treatment | | 29.5% | (22.9%, 36.1%) |
| difference (95% CI)^{e} | | | |

| | |
|---|---|
| p-value^{f} | <0.001 |

Note:
Includes only subjects with modified Mayo score 5-9 at induction baseline.
^{a}Symptomatic remission is defined as a stool frequency subscore of 0 or 1 and a rectal bleeding subscore of 0, where the stool frequency subscore has not increased from induction baseline.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated time point were considered not to be in symptomatic remission at the designated time point. For subjects who discontinued study agent due to COVID-19 related reasons (excluding COVID-19 infection) or regional crisis (ICE 4) prior to the designated time point, their observed values will be used, if available. Subjects who experienced ICE 5 (discontinued study agent due to reasons other than those in ICEs 3 and 4) prior to the designated time point were considered not to be in symptomatic remission at the designated time point.
^{c}Nonresponder Imputation for Missing Data: After accounting for ICEs, subjects who were missing one or more Mayo subscore pertaining to this endpoint (stool frequency and/or rectal bleeding) at the designated time point were considered not to be in symptomatic remission.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-Failure status (Yes/No) and concomitant use of corticosteroids at baseline (Yes/No).

TABLE 11
Summary of Key Efficacy Endpoints Among Subjects
Who Were ADT Non-failure; Full Analysis Set
(Study CNTO1959UCO3001; Induction 2)

| | Placebo IV | Guselkumab 200 mg IV | |
|---|---|---|---|
| Analysis set: Full | 280 | 421 | |
| Subjects who were | 144 (51.4%) | 213 | (50.6%) |
| ADT non-failure | | | |
| Subjects in clinical | 17 (11.8%) | 69 | (32.4%) |
| remission at | | | |
| Week I-12^{a1,b,c,d} | | | |
| 95% CI for treatment | (6.5%, 17.1%) | (26.1%, 38.7%) | |
| proportion^{e} | | | |
| Adjusted treatment | | 20.6% | (12.4%, 28.8%) |
| difference (95% CI)^{f} | | | |
| p-value^{g} | | <0.001 | |
| Subjects in symptomatic | 38 (26.4%) | 130 | (61.0%) |
| remission at | | | |
| Week I-12^{a2,b,c,d} | | | |
| 95% CI for treatment | (19.2%, 33.6%) | (54.5%, 67.6%) | |
| proportion^{e} | | | |
| Adjusted treatment | | 34.6% | (24.9%, 44.4%) |
| difference (95% CI)^{f} | | | |
| p-value^{g} | | <0.001 | |
| Subjects in symptomatic | 39 (27.1%) | 103 | (48.4%) |
| remission at | | | |
| Week I-8^{a2,b,c,d} | | | |
| 95% CI for treatment | (19.8%, 34.3%) | (41.6%, 55.1%) | |
| proportion^{e} | | | |
| Adjusted treatment | | 21.2% | (11.4%, 31.1%) |
| difference (95% CI)^{f} | | | |
| p-value^{g} | | <0.001 | |
| Subjects in symptomatic | 24 (16.7%) | 64 | (30.0%) |
| remission at | | | |
| Week I-4^{a2,b,c,d} | | | |
| 95% CI for treatment | (10.6%, 22.8%) | (23.9%, 36.2%) | |
| proportion^{e} | | | |
| Adjusted treatment | | 13.4% | (4.7%, 22.0%) |
| difference (95% CI)^{f} | | | |
| p-value^{g} | | 0.004 | |
| Subjects in symptomatic | 19 (13.2%) | 35 | (16.4%) |
| remission at | | | |
| Week I-2^{a2,b,c,d} | | | |
| 95% CI for treatment | (7.7%, 18.7%) | (11.5%, 21.4%) | |
| proportion^{e} | | | |
| Adjusted treatment | | 3.2% | (−4.2%, 10.6%) |
| difference (95% CI)^{f} | | | |
| p-value^{g} | | 0.408 | |
| Subjects with endoscopic | 24 (16.7%) | 82 | (38.5%) |
| healing at | | | |
| Week I-12^{a3,b,c,d} | | | |
| 95% CI for treatment | (10.6%, 22.8%) | (32.0%, 45.0%) | |
| proportion^{e} | | | |
| Adjusted treatment | | 21.8% | (12.9%, 30.8%) |
| difference (95% CI)^{f} | | | |
| p-value^{g} | | <0.001 | |
| Subjects in clinical | 50 (34.7%) | 152 | (71.4%) |
| response at | | | |
| Week I-12^{a4,b,c,d} | | | |
| 95% CI for treatment | (26.9%, 42.5%) | (65.3%, 77.4%) | |
| proportion^{e} | | | |
| Adjusted treatment | | 36.7% | (26.8%, 46.5%) |
| difference (95% CI)^{f} | | | |
| p-value^{g} | | <0.001 | |
| Subjects in IBDQ | 50 (34.7%) | 134 | (62.9%) |
| remission at | | | |
| Week I-12^{a5,b,c,d} | | | |
| 95% CI for treatment | (26.9%, 42.5%) | (56.4%, 69.4%) | |
| proportion^{e} | | | |
| Adjusted treatment | | 28.2% | (18.1%, 38.3%) |
| difference (95% CI)^{f} | | | |
| p-value^{g} | | <0.001 | |
| Subjects with histologic- | 15 (10.4%) | 70 | (32.9%) |
| endoscopic mucosal | | | |
| healing at | | | |
| Week I-12^{a6,b,c,d} | | | |
| 95% CI for treatment | (5.4%, 15.4%) | (26.6%, 39.2%) | |
| proportion^{e} | | | |
| Adjusted treatment | | 22.4% | (14.4%, 30.5%) |
| difference (95% CI)^{f} | | | |
| p-value^{g} | | <0.001 | |
| Subjects in fatigue | 42 (29.2%) | 93 | (43.7%) |
| response at | | | |
| Week I-12^{a7,b,c,d} | | | |
| 95% CI for treatment | (21.7%, 36.6%) | (37.0%, 50.3%) | |
| proportion^{e} | | | |
| Adjusted treatment | | 14.5% | (4.5%, 24.5%) |
| difference (95% CI)^{f} | | | |
| p-value^{g} | | 0.006 | |
| Subjects with endoscopic | 11 (7.6%) | 45 | (21.1%) |
| normalization at | | | |
| Week I-12^{a8,b,c,d} | | | |
| 95% CI for treatment | (3.3%, 12.0%) | (15.6%, 26.6%) | |
| proportion^{e} | | | |
| Adjusted treatment | | 13.5% | (6.5%, 20.4%) |
| difference (95% CI)^{f} | | | |
| p-value^{g} | | <0.001 | |
| Subjects with histologic | 39 (27.1%) | 117 | (54.9%) |
| healing at | | | |
| Week I-12^{a9,b,c,d} | | | |
| 95% CI for treatment | (19.8%, 34.3%) | (48.2%, 61.6%) | |
| proportion^{e} | | | |
| Adjusted treatment | | 27.9% | (18.0%, 37.7%) |
| difference (95% CI)^{f} | | | |
| p-value^{g} | | <0.001 | |
| Subjects in histologic | 34 (23.6%) | 104 | (48.8%) |
| remission at | | | |
| Week I-12^{a10,b,c,d} | | | |
| 95% CI for treatment | (16.7%, 30.5%) | (42.1%, 55.5%) | |
| proportion^{e} | | | |
| Adjusted treatment | | 25.3% | (15.6%, 34.9%) |
| difference (95% CI)^{f} | | | |
| p-value^{g} | | <0.001 | |
| Subjects with histologic- | 14 (9.7%) | 64 | (30.0%) |
| endoscopic mucosal | | | |
| healing (alternative | | | |
| definition 1) | | | |
| at Week I-12^{a11,b,c,d} | | | |
| 95% CI for treatment | (4.9%, 14.6%) | (23.9%, 36.2%) | |
| proportion^{e} | | | |
| Adjusted treatment | | 20.3% | (12.5%, 28.2%) |
| difference (95% CI)^{f} | | | |
| p-value^{g} | | <0.001 | |
| Subjects with deep | 8 (5.6%) | 41 | (19.2%) |
| histologic-endoscopic | | | |
| mucosal healing | | | |
| at Week I-12^{a12,b,c,d} | | | |
| 95% CI for treatment | (1.8%, 9.3%) | (14.0%, 24.5%) | |
| proportion^{e} | | | |
| Adjusted treatment | | 13.7% | (7.2%, 20.1%) |
| difference (95% CI)^{f} | | | |

| | |
|---|---|
| p-value^{g} | <0.001 |

Key: ADT = advanced therapy; IBDQ = Inflammatory Bowel Disease Questionnaire; PROMIS = Patient-Reported Outcomes Measurement Information System.
Note:
Includes only subjects with modified Mayo score 5-9 at induction baseline.
^{a1}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{a2}Symptomatic remission is defined as a stool frequency subscore of 0 or 1 and a rectal bleeding subscore of 0, where the stool frequency subscore has not increased from induction baseline.
^{a3}Endoscopic healing is defined as an endoscopy subscore of 0 or 1 with no friability present on the endoscopy.
^{a4}Clinical response is defined as a decrease from induction baseline in the modified Mayo score by ≥30% and ≥2 points, with either a ≥1-point decrease from baseline in the rectal bleeding subscore or a rectal bleeding subscore of 0 or 1.
^{a5}IBDQ remission is defined as a total IBDQ score ≥170.
^{a6}Histologic-endoscopic mucosal healing is defined as achieving a combination of histologic healing and endoscopic healing.
^{a7}Fatigue response is defined as ≥7-point improvement from baseline in the PROMIS Fatigue Short Form 7a.
^{a8}Endoscopic normalization is defined as an endoscopy subscore of 0.
^{a9}Histologic healing is defined as neutrophil infiltration in <5% of crypts, no crypt destruction, and no erosions, ulcerations or granulation tissue according to the Geboes grading system.
^{a10}Histologic remission is defined as absence of neutrophils from mucosa (both lamina propria and epithelium), no crypt destruction, and no erosions, ulcerations or granulation tissue according to the Geboes grading system.
^{a11}Histologic-endoscopic mucosal healing (alternative definition 1) is defined as achieving a combination of histologic remission and endoscopic healing.
^{a12}Deep histologic-endoscopic mucosal healing is defined as achieving a combination of endoscopic normalization and histologic remission.
^{b}Denominator is subjects who were ADT non-failure.
^{c}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint were considered not to have achieved any of the key efficacy endpoints shown at the designated timepoint. For subjects who discontinued study agent due to COVID-19 related reasons (excluding COVID-19 infection) or regional crisis (ICE 4) prior to the designated timepoint, their observed values will be used, if available. Subjects who experienced ICE 5 (discontinued study agent due to reasons other than those in ICEs 3 and 4) prior to the designated timepoint were considered not to have achieved any of the key efficacy endpoints shown at the designated timepoint.
^{d}Nonresponder Imputation for Missing Data: After accounting for ICEs, subjects who were missing one or more of the components pertaining to an endpoint at the designated timepoint were considered not to have achieved the endpoint. Subjects who had an unevaluable biopsy (i.e., a biopsy that was collected, but could not be assessed due to sample preparation or technical errors) were considered not to have achieved the histology endpoints.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) chi-square test, stratified by concomitant use of corticosteroids at baseline (Yes/No).

TABLE 12
Summary of Key Efficacy Endpoints Among Subjects
Who Were Biologic-failure; Full Analysis Set
(Study CNTO1959UCO3001; Induction 2)

| | Placebo IV | Guselkumab 200 mg IV | |
|---|---|---|---|
| Analysis set: Full | 280 | 421 | |
| Subjects who were | 135 (48.2%) | 203 | (48.2%) |
| biologic-failure | | | |
| Subjects in clinical | 5 (3.7%) | 24 | (11.8%) |
| remission at | | | |
| Week I-12^{a1,b,c,d} | | | |
| 95% CI for treatment | (0.5%, 6.9%) | (7.4%, 16.3%) | |
| proportion^{e} | | | |
| Adjusted treatment | | 8.1% | (2.7%, 13.6%) |
| difference (95% CI)^{f} | | | |
| p-value^{g} | | 0.009 | |
| Subjects in symptomatic | 19 (14.1%) | 77 | (37.9%) |
| remission at | | | |
| Week I-12^{a2,b,c,d} | | | |
| 95% CI for treatment | (8.2%, 19.9%) | (31.3%, 44.6%) | |
| proportion^{e} | | | |
| Adjusted treatment | | 23.8% | (15.0%, 32.7%) |
| difference (95% CI)^{f} | | | |
| p-value^{g} | | <0.001 | |
| Subjects in symptomatic | 18 (13.3%) | 61 | (30.0%) |
| remission at | | | |
| Week I-8^{a2,b,c,d} | | | |
| 95% CI for treatment | (7.6%, 19.1%) | (23.7%, 36.4%) | |
| proportion^{e} | | | |
| Adjusted treatment | | 16.7% | (8.2%, 25.2%) |
| difference (95% CI)^{f} | | | |
| p-value^{g} | | <0.001 | |
| Subjects in symptomatic | 11 (8.1%) | 28 | (13.8%) |
| remission at | | | |
| Week I-4^{a2,b,c,d} | | | |
| 95% CI for treatment | (3.5%, 12.8%) | (9.0%, 18.5%) | |
| proportion^{e} | | | |
| Adjusted treatment | | 5.6% | (−0.9%, 12.2%) |
| difference (95% CI)^{f} | | | |
| p-value^{g} | | 0.114 | |
| Subjects in symptomatic | 7 (5.2%) | 15 | (7.4%) |
| remission at | | | |
| Week I-2^{a2,b,c,d} | | | |
| 95% CI for treatment | (1.4%, 8.9%) | (3.8%, 11.0%) | |
| proportion^{e} | | | |
| Adjusted treatment | | 2.2% | (−2.9%, 7.4%) |
| difference (95% CI)^{f} | | | |
| p-value^{g} | | 0.423 | |
| Subjects with endoscopic | 7 (5.2%) | 29 | (14.3%) |
| healing at | | | |
| Week I-12^{a3,b,c,d} | | | |
| 95% CI for treatment | (1.4%, 8.9%) | (9.5%, 19.1%) | |
| proportion^{e} | | | |
| Adjusted treatment | | 9.1% | (3.1%, 15.2%) |
| difference (95% CI)^{f} | | | |
| p-value^{g} | | 0.008 | |
| Subjects in clinical | 27 (20.0%) | 105 | (51.7%) |
| response at | | | |
| Week I-12^{a4,b,c,d} | | | |
| 95% CI for treatment | (13.3%, 26.7%) | (44.9%, 58.6%) | |
| proportion^{e} | | | |
| Adjusted treatment | | 31.7% | (22.1%, 41.3%) |
| difference (95% CI)^{f} | | | |
| p-value^{g} | | <0.001 | |
| Subjects in IBDQ | 32 (23.7%) | 80 | (39.4%) |
| remission at | | | |
| Week I-12^{a5,b,c,d} | | | |
| 95% CI for treatment | (16.5%, 30.9%) | (32.7%, 46.1%) | |
| proportion^{e} | | | |
| Adjusted treatment | | 15.7% | (5.9%, 25.5%) |
| difference (95% CI)^{f} | | | |
| p-value^{g} | | 0.003 | |
| Subjects with histologic- | 6 (4.4%) | 26 | (12.8%) |
| endoscopic mucosal | | | |
| healing at | | | |
| Week I-12^{a6,b,c,d} | | | |
| 95% CI for treatment | (1.0%, 7.9%) | (8.2%, 17.4%) | |
| proportion^{e} | | | |
| Adjusted treatment | | 8.4% | (2.6%, 14.1%) |
| difference (95% CI)^{f} | | | |
| p-value^{g} | | 0.010 | |
| Subjects in fatigue | 17 (12.6%) | 76 | (37.4%) |
| response at | | | |
| Week I-12^{a7,b,c,d} | | | |
| 95% CI for treatment | (7.0%, 18.2%) | (30.8%, 44.1%) | |
| proportion^{e} | | | |
| Adjusted treatment | | 24.9% | (16.2%, 33.5%) |
| difference (95% CI)^{f} | | | |
| p-value^{g} | | <0.001 | |
| Subjects with endoscopic | 3 (2.2%) | 18 | (8.9%) |
| normalization at | | | |
| Week I-12^{a8,b,c,d} | | | |
| 95% CI for treatment | (0.0%, 4.7%) | (5.0%, 12.8%) | |
| proportion^{e} | | | |
| Adjusted treatment | | 6.6% | (2.0%, 11.3%) |
| difference (95% CI)^{f} | | | |
| p-value^{g} | | 0.013 | |
| Subjects with histologic | 21 (15.6%) | 67 | (33.0%) |
| healing at | | | |
| Week I-12^{a9,b,c,d} | | | |
| 95% CI for treatment | (9.4%, 21.7%) | (26.5%, 39.5%) | |
| proportion^{e} | | | |
| Adjusted treatment | | 17.4% | (8.6%, 26.3%) |
| difference (95% CI)^{f} | | | |
| p-value^{g} | | <0.001 | |
| Subjects in histologic | 18 (13.3%) | 59 | (29.1%) |
| remission at | | | |
| Week I-12^{a10,b,c,d} | | | |
| 95% CI for treatment | (7.6%, 19.1%) | (22.8%, 35.3%) | |
| proportion^{e} | | | |
| Adjusted treatment | | 15.7% | (7.3%, 24.2%) |
| difference (95% CI)^{f} | | | |
| p-value^{g} | | <0.001 | |
| Subjects with histologic- | 6 (4.4%) | 26 | (12.8%) |
| endoscopic mucosal | | | |
| healing (alternative | | | |
| definition 1) | | | |
| at Week I-12^{a11,b,c,d} | | | |
| 95% CI for treatment | (1.0%, 7.9%) | (8.2%, 17.4%) | |
| proportion^{e} | | | |
| Adjusted treatment | | 8.4% | (2.6%, 14.1%) |
| difference (95% CI)^{f} | | | |
| p-value^{g} | | 0.010 | |
| Subjects with deep | 3 (2.2%) | 15 | (7.4%) |
| histologic-endoscopic | | | |
| mucosal healing | | | |
| at Week I-12^{a12,b,c,d} | | | |
| 95% CI for treatment | (0.0%, 4.7%) | (3.8%, 11.0%) | |
| proportion^{e} | | | |
| Adjusted treatment | | 5.2% | (0.8%, 9.5%) |
| difference (95% CI)^{f} | | | |

| | |
|---|---|
| p-value^{g} | 0.039 |

Key: IBDQ = Inflammatory Bowel Disease Questionnaire; PROMIS = Patient-Reported Outcomes Measurement Information System.
Note:
Includes only subjects with modified Mayo score 5-9 at induction baseline.
^{a1}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{a2}Symptomatic remission is defined as a stool frequency subscore of 0 or 1 and a rectal bleeding subscore of 0, where the stool frequency subscore has not increased from induction baseline.
^{a3}Endoscopic healing is defined as an endoscopy subscore of 0 or 1 with no friability present on the endoscopy.
^{a4}Clinical response is defined as a decrease from induction baseline in the modified Mayo score by ≥30% and ≥2 points, with either a ≥1-point decrease from baseline in the rectal bleeding subscore or a rectal bleeding subscore of 0 or 1.
^{a5}IBDQ remission is defined as a total IBDQ score ≥170.
^{a6}Histologic-endoscopic mucosal healing is defined as achieving a combination of histologic healing and endoscopic healing.
^{a7}Fatigue response is defined as ≥7-point improvement from baseline in the PROMIS Fatigue Short Form 7a.
^{a8}Endoscopic normalization is defined as an endoscopy subscore of 0.
^{a9}Histologic healing is defined as neutrophil infiltration in <5% of crypts, no crypt destruction, and no erosions, ulcerations or granulation tissue according to the Geboes grading system.
^{a10}Histologic remission is defined as absence of neutrophils from mucosa (both lamina propria and epithelium), no crypt destruction, and no erosions, ulcerations or granulation tissue according to the Geboes grading system.
^{a11}Histologic-endoscopic mucosal healing (alternative definition 1) is defined as achieving a combination of histologic remission and endoscopic healing.
^{a12}Deep histologic-endoscopic mucosal healing is defined as achieving a combination of endoscopic normalization and histologic remission.
^{b}Denominator is subjects who were biologic-failure.
^{c}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint were considered not to have achieved any of the key efficacy endpoints shown at the designated timepoint. For subjects who discontinued study agent due to COVID-19 related reasons (excluding COVID-19 infection) or regional crisis (ICE 4) prior to the designated timepoint, their observed values will be used, if available. Subjects who experienced ICE 5 (discontinued study agent due to reasons other than those in ICEs 3 and 4) prior to the designated timepoint were considered not to have achieved any of the key efficacy endpoints shown at the designated timepoint.
^{d}Nonresponder Imputation for Missing Data: After accounting for ICEs, subjects who were missing one or more of the components pertaining to an endpoint at the designated timepoint were considered not to have achieved the endpoint. Subjects who had an unevaluable biopsy (i.e., a biopsy that was collected, but could not be assessed due
to sample preparation or technical errors) were considered not to have achieved the histology endpoints.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) chi-square test, stratified by concomitant use of corticosteroids at baseline (Yes/No).

TABLE 13
Overall Summary of Treatment-emergent Adverse
Events Through Week I-12; Safety Analysis
Set (Study CNTO1959UCO3001; Induction 2)

| | | | Guselkumab | |
|---|---|---|---|---|
| | Placebo IV | | 200 mg IV | |
| Analysis set: Safety | 280 | | 421 | |
| Average duration of follow-up (weeks) | 11.9 | | 12.1 | |
| Average exposure (number of | 2.9 | | 2.9 | |
| administrations) | | | | |
| Subjects with 1 or more: | | | | |
| Adverse events | 136 | (48.6%) | 204 | (48.5%) |
| Serious adverse events | 21 | (7.5%) | 12 | (2.9%) |
| Adverse events leading to | 12 | (4.3%) | 6 | (1.4%) |
| discontinuation of study agent | | | | |
| Reasonably-related adverse events^{a} | 24 | (8.6%) | 55 | (13.1%) |
| Infections^{b} | 42 | (15.0%) | 66 | (15.7%) |
| Serious infections^{b} | 1 | (0.4%) | 3 | (0.7%) |
| Adverse events within 1 hour of | 1 | (0.4%) | 6 | (1.4%) |
| infusion | | | | |
| Adverse events leading to death | 2 | (0.7%) | 1 | (0.2%) |

Note:
Includes only subjects with modified Mayo score 5-9 at induction baseline.
^{a}An adverse event that is assessed by the investigator as possibly, probably, or very likely related to study agent or if the relationship to study agent is missing.
^{b}Infections were defined as any adverse event which was coded to the MedDRA system organ class ‘Infections and infestations’.

TABLE 14
Number of Subjects in Clinical/Symptomatic
Response Through Week I-12; Full Analysis
Set (Study CNTO1959UCO3001; Induction 2)

| | Placebo IV | Guselkumab 200 mg IV | |
|---|---|---|---|
| Analysis set: Full | 280 | 421 | |
| Week I-2 | | | |
| N | 280 | 421 | |
| Subjects in symptomatic | 66 (23.6%) | 143 | (34.0%) |
| response^{a,b,c} | | | |
| 95% CI for treatment | (18.6%, 28.5%) | (29.4%, 38.5%) | |
| proportion^{d} | | | |
| Adjusted treatment | | 10.7% | (4.0%, 17.3%) |
| difference (95% CI)^{e} | | | |
| p-value^{f} | | 0.002 | |
| Week I-4 | | | |
| N | 280 | 421 | |
| Subjects in symptomatic | 84 (30.0%) | 224 | (53.2%) |
| response^{a,b,c} | | | |
| 95% CI for treatment | (24.6%, 35.4%) | (48.4%, 58.0%) | |
| proportion^{d} | | | |
| Adjusted treatment | | 23.4% | (16.4%, 30.4%) |
| difference (95% CI)^{e} | | | |
| p-value^{f} | | <0.001 | |
| Week I-8 | | | |
| N | 280 | 421 | |
| Subjects in symptomatic | 111 (39.6%) | 278 | (66.0%) |
| response^{a,b,c} | | | |
| 95% CI for treatment | (33.9%, 45.4%) | (61.5%, 70.6%) | |
| proportion^{d} | | | |
| Adjusted treatment | | 26.7% | (19.5%, 33.9%) |
| difference (95% CI)^{e} | | | |
| p-value^{f} | | <0.001 | |
| Week I-12 | | | |
| N | 280 | 421 | |
| Subjects in symptomatic | 98 (35.0%) | 302 | (71.7%) |
| response^{a,b,c} | | | |
| 95% CI for treatment | (29.4%, 40.6%) | (67.4%, 76.0%) | |
| proportion^{d} | | | |
| Adjusted treatment | | 37.0% | (30.1%, 43.9%) |
| difference (95% CI)^{e} | | | |

| | |
|---|---|
| p-value^{f} | <0.001 |

Note:
Includes only subjects with modified Mayo score 5-9 at induction baseline.
^{a}Symptomatic response is defined as a decrease from induction baseline in the symptomatic Mayo score by ≥30% and ≥1 point, with either a ≥1 point decrease from baseline in the rectal bleeding subscore or a rectal bleeding subscore of 0 or 1. The symptomatic Mayo score is defined as the sum of the stool frequency and the rectal bleeding subscores.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated time point were considered not to be in symptomatic response at the designated time point. For subjects who discontinued study agent due to COVID-19 related reasons (excluding COVID-19 infection) or regional crisis in Russia and Ukraine (ICE 4) prior to the designated time point, their observed values were used, if available. Subjects who experienced ICE 5 (discontinued study agent due to reasons other than those in ICEs 3 and 4) prior to the designated time point were considered not to be in symptomatic response at the designated time point.
^{c}Nonresponder Imputation for Missing Data: After accounting for ICEs, subjects who were missing one or more Mayo subscore pertaining to this endpoint (stool frequency and/or rectal bleeding) at the designated time point were considered not to be in symptomatic response.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-failure status (Yes/No) and concomitant use of corticosteroids at baseline (Yes/No).

TABLE 15
Symptomatic outcomes at Week 12

| | | | Treatment Difference ^{d} | |
|---|---|---|---|---|
| | Placebo IV | Guselkumab 200 mg IV | (95% CI) | |
| Primary analysis population, N | 280 | 421 | | |
| Symptomatic response,^{a,b} | 35.0% (29.4%, 40.6%) | 71.7% (67.4%, 76.0%) | 37.0% | (30.1%, 43.9%)*** |
| proportion of patients (95% CI) | | | | |
| Symptomatic remission,^{a,c} | 20.7% (16.0%, 25.5%) | 49.9% (45.1%, 54.7%) | 29.4% (22.8%, 36.0%), | |
| proportion of patients (95% CI) | | | P < 0.001 | |
| Stool frequency subscore of 0 or 1^{a} | 31.8% (26.3%, 37.2%) | 60.1% (55.4%, 64.8%) | 28.5% | (21.5%, 35.6%)*** |
| proportion of patients (95% CI) | | | | |
| Rectal bleeding subscore of 0^{a} | 28.6% (23.3%, 33.9%) | 64.6% (60.0%, 69.2%) | 36.2% | (29.3%, 43.2%)*** |
| proportion of patients (95% CI) | | | | |
| Absolute number of stools per day,^{a} | N = 274 | N = 420 | −1.78 | (−2.20, −1.37) |
| mean change from baseline (95% CI) | −1.36 (−1.7, −1.0) | −3.15 (−3.5, −2.8) | | |
| Rectal bleeding subscore,^{a} | N = 274 | N = 420 | −0.7 | (−0.8, −0.5) |

| | | |
|---|---|---|
| Mean change from baseline (95% CI) | −0.6 (−0.7, −0.5) | −1.2 (−1.3, −1.1) |

***Nominal p < 0.001.
^{a}Patients who had a prohibited change in UC medication, an ostomy or colectomy, or discontinued study agent due to lack of efficacy or an adverse event of worsening of UC prior to the designated time point were considered not to have achieved the endpoint for binary endpoints and had baseline observation carried forward for continuous endpoints. Data after discontinuation of study agent due to COVID-19 related reasons (excluding COVID-19 infection) were considered to be missing. Patients who were missing one or more components pertaining to a specified endpoint were considered not to have achieved the endpoint for binary endpoints and had baseline observation carried forward for continuous endpoints. The p-values for binary endpoints were based on the Cochran-Mantel-Haenszel (CMH) chi-square test. The p-values for continuous endpoints were based on a Mixed-Effect Model Repeated Measures.
^{b}Symptomatic response was defined a decrease from induction baseline in the symptomatic Mayo score (sum of the stool frequency and the rectal bleeding subscores) by ≥30% and ≥1 point, with either a ≥1 point decrease from baseline in the rectal bleeding subscore or a rectal bleeding subscore of 0 or 1.
^{c}Symptomatic remission was defined as a stool frequency subscore of 0 or 1 and not increased from induction baseline, and a rectal bleeding subscore of 0.
^{d}Treatment differences shown for symptomatic response, symptomatic remission, stool frequency/rectal bleeding subscores of 0 or 1 are adjusted treatment differences; treatment differences shown for change from baseline in absolute stool number and rectal bleeding subscore are in LS means.

TABLE 16
Safety summary through final safety visit (Week 32)

| | | | | | Cross-over | | | | Combined | | | |
|---|---|---|---|---|---|---|---|---|---|---|---|---|
| | IV Induction | | | | PBO IV → | | GUS 200 mg IV → | | Combined | | | |
| | PBO IV^{a} | | GUS 200 mg IV^{a} | | GUS 200 mg IV^{b} | | GUS 200 mg SC^{b} | | GUS IV^{c} | | All GUS^{d} | |
| Safety analysis set, N | 280 | | 421 | | 165 | | 125 | | 586 | | 586 | |
| Average duration of follow- | 12.1 | | 12.3 | | 13.9 | | 14.6 | | 12.8 | | 15.9 | |
| up (weeks) | | | | | | | | | | | | |
| Average exposure (number | 2.9 | | 2.9 | | 2.9 | | 2.8 | | 2.9 | | 3.5 | |
| of administrations) | | | | | | | | | | | | |
| Deaths | 2 | (0.7%) | 1 | (0.2%) | 0 | | 0 | | 1 | (0.2%) | 1 | (0.2%) |
| Patients with ≥1: | | | | | | | | | | | | |
| Adverse events | 138 | (49.3%) | 208 | (49.4%) | 65 | (39.4%) | 56 | (44.8%) | 273 | (46.6%) | 292 | (49.8%) |
| Serious adverse events | 20 | (7.1%) | 12 | (2.9%) | 3 | (1.8%) | 3 | (2.4%) | 15 | (2.6%) | 18 | (3.1%) |
| Adverse events leading | 11 | (3.9%) | 7 | (1.7%) | 7 | (4.2%) | 5 | (4.0%) | 14 | (2.4%) | 19 | (3.2%) |
| to discontinuation of | | | | | | | | | | | | |
| study agent | | | | | | | | | | | | |
| Infections^{e} | 43 | (15.4%) | 67 | (15.9%) | 26 | (15.8%) | 18 | (14.4%) | 93 | (15.9%) | 104 | (17.7%) |
| Serious infections^{e} | 1 | (0.4%) | 3 | (0.7%) | 1 | (0.6%) | 2 | (1.6%) | 4 | (0.7%) | 6 | (1.0%) |
| Adverse events within 1 | 1 | (0.4%) | 6 | (1.4%) | 1 | (0.6%) | 3 | (2.4%) | 7 | (1.2%) | 10 | (1.7%) |
| hour of infusion | | | | | | | | | | | | |

| | | | | | | | | | | |
|---|---|---|---|---|---|---|---|---|---|---|
| Injection-site reactions^{f} | 0 | 0 | 1 | (0.6%) | 1 | (0.8%) | 1 | (0.2%) | 2 | (0.3%) |

Note:
Includes only pts with modified Mayo score 5-9 at induction baseline.
^{a}Includes data up to Week 12 for subjects who received treatment at Week 12. Includes all data through final safety visit for subjects who did not receive treatment at Week 12.
^{b}Includes data from Week 12 onward.
^{c}From the first guselkumab IV dose onward; for subjects who received guselkumab 200 mg SC at Week 12, includes data up to Week 12.
^{d}From the first guselkumab dose onward.
^{e}Infections were defined as any adverse event which was coded to the MedDRA system organ class ‘Infections and infestations’.
^{f}Injection-site reactions as assessed by the investigator.

TABLE 17
Summary of Histologic and Histo-Endoscopic Outcomes at Week 12

| | | | Adjusted treatment |
|---|---|---|---|
| | | Guselkumab | difference (95% CI) |
| | Placebo IV | 200 mg IV | p-value |
| Full analysis set, N | 280 | 421 | — |
| Histologic improvement, n (%) | 60 (21.4) | 189 (44.9) | 23.7 (17.0, 30.3) |
| (Neutrophil infiltration in <5% of crypts, no crypt | | | Nominal p < 0.001 |
| destruction, and no erosions, ulcerations or | | | |
| granulation tissue according to the Geboes grading | | | |
| system [ie, Geboes histologic score ≤3.1]) | | | |
| Histologic remission, n (%) | 52 (18.6) | 168 (39.9) | 21.5 (15.1, 27.9) |
| (Absence of neutrophils from the mucosa [both | | | Nominal p < 0.001 |
| lamina propria and epithelium], no crypt destruction, | | | |
| and no erosions, ulcerations or granulation tissue | | | |
| according to the Geboes grading system [ie, Geboes | | | |
| histologic score ≤2 B.0])^{a} | | | |
| Histologic remission by alternative definition, n (%) | 52 (18.6) | 168 (39.9) | 21.5 (15.1, 27.9) |
| (Nancy Histological Index ≤1) | | | Nominal p < 0.001 |
| Histo-endoscopic mucosal improvement, n (%) | 21 (7.5) | 99 (23.5) | 16.2 (11.1, 21.2) |
| (Achieving a combination of histologic | | | p < 0.001 |
| improvement and endoscopic improvement | | | |
| [endoscopy subscore of 0 or 1 with no friability | | | |
| present on the endoscopy]) | | | |
| Deep histo-endoscopic mucosal improvement, n (%) | 11 (3.9) | 57 (13.5) | 9.8 (5.8, 13.7) |
| (Achieving a combination of histologic remission | | | Nominal p < 0.001 |
| [Geboes histologic score ≤2 B.0] and endoscopic | | | |
| normalization [endoscopy subscore of 0]) | | | |

^{a}This definition is equivalent to histologic remission by alternative definition using the Robarts Histopathology Index (RHI ≤3, with subscores of 0 for lamina propria neutrophils and neutrophils in the epithelium and without ulcers or erosion).
Note:
Patients who had a prohibited change in UC medication, an ostomy or colectomy, or discontinued study agent due to lack of therapeutic effect or due to an AE of worsening of UC, or due to other reasons except for COVID-19 related reasons (excluding COVID-19 infection) or regional crisis in Russia and Ukraine prior to Week 12 were considered not to have achieved the endpoint. Patients who had an unevaluable biopsy (i.e., a biopsy that was collected, but could not be assessed due to sample preparation or technical errors) or were missing the endoscopy subscore (if applicable) or any of the histology components pertaining to this endpoint (i.e., assessment of neutrophils in epithelium, crypt destruction, or erosions or ulcerations or granulations) at Week 12 were considered not to have achieved the endpoint. The adjusted treatment difference and confidence intervals were based on the Wald statistic with Cochran-Mantel-Haenszel weight. The p-values were based on the Cochran-Mantel-Haenszel (CMH) chi-square test, stratified by ADT-Failure status (Yes/No) and concomitant use of corticosteroids at baseline (Yes/No).

TABLE 18
Change from baseline in CRP and FeCal through Week 12 among patients with
elevated CRP (>3 mg/L) or FeCal (>250 mg/kg) at baseline

| | Placebo IV | | Guselkumab 200 mg IV | |
|---|---|---|---|---|
| CRP (mg/L) | | | | |
| Baseline, N | 160 | | 248 | |
| Median [IQR] | 8.02 | [5.40; 16.85] | 9.26 | [5.31; 18.05] |
| Wk 4, N | 158 | | 245 | |
| Median change from baseline [IQR] | −2.06 | [−5.70; 1.58] | −3.35 | [−8.60; − 0.23]*** |
| Wk 8, N | 156 | | 239 | |
| Median change from baseline [IQR] | −1.75 | [−5.68; 1.46] | −3.80 | [−10.77; − 0.19]*** |
| Wk 12, N | 153 | | 239 | |
| Median change from baseline [IQR] | −0.51 | [−4.64; 2.72] | −3.99 | [−11.46; − 0.85]*** |
| FeCal (mg/kg) | | | | |
| Baseline, N | 225 | | 333 | |
| Median [IQR] | 1743.00 | [1120.00; 3395.00] | 1787.00 | [920.00; 4009.00] |
| Wk 4, N | 213 | | 308 | |
| Median change from baseline [IQR] | −227.00 | [−1041.00; 658.00] | −603.00 | [−1866.00; 229.50]*** |
| Wk 12, N | 201 | | 293 | |
| Median change from baseline [IQR] | −86.00 | [−1254.00; 504.00] | −800.00 | [−2532.00; 0.00]*** |

***Nominal P ≤ 0.001. Patients who had a prohibited change in UC medication, an ostomy or colectomy, or discontinued study agent due to lack of efficacy or an AE of worsening of UC or other reasons except for COVID-19 related reasons (excluding COVID-19 infection) or regional crisis in Russia and Ukraine prior to the designated timepoint had their baseline value carried forward from the time of the event onward. The p-values of treatment comparison were based on the mixed-effect model repeated measures with CRP and FeCal values being log-transformed.

TABLE 19
Number of Subjects in Symptomatic Remission at Week I-1;
Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-1 | | | |
| N | 280 | 421 | |
| Subjects in symptomatic | 16 (5.7%) | 36 | (8.6%) |
| remission^{a,b,c} | | | |
| 95% CI for treatment | (3.0%, 8.4%) | (5.9%, 11.2%) | |
| proportion^{d} | | | |
| Adjusted treatment | | | |
| difference (95% CI)^{e} | | 3.0% | (−0.8%, 6.8%) |

| | |
|---|---|
| p-value^{f} | 0.135 |

Note:
Includes only subjects with modified Mayo score 5-9 at induction baseline.
^{a}Symptomatic remission is defined as a stool frequency subscore of 0 or 1 and a rectal bleeding subscore of 0, where the stool frequency subscore has not increased from induction baseline.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated time point were considered not to be in symptomatic remission at the designated time point. For subjects who discontinued study agent due to COVID-19 related reasons (excluding COVID-19 infection) or regional crisis in Russia and Ukraine (ICE 4) prior to the designated time point, their observed values were used, if available. Subjects who experienced ICE 5 (discontinued study agent due to reasons other than those in ICEs 3 and 4) prior to the designated time point were considered not to be in symptomatic remission at the designated time point.
^{c}Nonresponder Imputation for Missing Data: After accounting for ICEs, subjects who were missing one or more Mayo subscore pertaining to this endpoint (stool frequency and/or rectal bleeding) at the designated time point were considered not to be in symptomatic remission.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-failure status (Yes/No) and concomitant use of corticosteroids at baseline (Yes/No).

TABLE 20
Number of Subjects in Symptomatic Response at Week I-1;
Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-1 | | | |
| N | 280 | 421 | |
| Subjects in symptomatic | 53 (18.9%) | 119 | (28.3%) |
| response^{a,b,c} | | | |
| 95% CI for treatment | (14.3%, 23.5%) | (24.0%, 32.6%) | |
| proportion^{d} | | | |
| Adjusted treatment | | 9.5% | (3.2%, 15.7%) |
| difference (95% CI)^{e} | | | |

| | |
|---|---|
| p-value^{f} | 0.004 |

Note:
Includes only subjects with modified Mayo score 5-9 at induction baseline.
^{a}Symptomatic response is defined as a decrease from induction baseline in the symptomatic Mayo score by ≥30% and ≥1 point, with either a ≥1 point decrease from baseline in the rectal bleeding subscore or a rectal bleeding subscore of 0 or 1. The symptomatic Mayo score is defined as the sum of the stool frequency and the rectal bleeding subscores.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated time point were considered not to be in symptomatic response at the designated time point. For subjects who discontinued study agent due to COVID-19 related reasons (excluding COVID-19 infection) or regional crisis in Russia and Ukraine (ICE 4) prior to the designated time point, their observed values were used, if available. Subjects who experienced ICE 5 (discontinued study agent due to reasons other than those in ICEs 3 and 4) prior to the designated time point were considered not to be in symptomatic response at the designated time point.
^{c}Nonresponder Imputation for Missing Data: After accounting for ICEs, subjects who were missing one or more Mayo subscore pertaining to this endpoint (stool frequency and/or rectal bleeding) at the designated time point were considered not to be in symptomatic response.
^{d}The confidence intervals for the proportion of subjects meeting the endpoint in each treatment group were based on the normal approximation confidence limits.
^{e}The adjusted treatment difference and confidence intervals were based on the Wald statistic with Cochran-Mantel-Haenszel weight.
^{f}The p-values were based on the Cochran-Mantel-Haenszel (CMH) chi-square test, stratified by ADT-failure status (Yes/No) and concomitant use of corticosteroids at baseline (Yes/No).

TABLE 21
Number of Subjects With a Stool Frequency Subscore
of 0 or 1 at Week I-1; Full Analysis Set (Study
CNTO1959UCO3001; Induction 2)

| | Placebo IV | Guselkumab 200 mg IV | |
|---|---|---|---|
| Analysis set: Full | 280 | 421 | |
| Week I-1 | | | |
| N | 280 | 421 | |
| Subjects with a stool | 45 (16.1%) | 96 | (22.8%) |
| frequency subscore | | | |
| of 0 or 1^{b,c} | | | |
| 95% CI for treatment | (11.8%, 20.4%) | (18.8%, 26.8%) | |
| proportion^{a} | | | |
| Adjusted treatment | | 6.9% | (1.1%, 12.7%) |
| difference (95% CI)^{d} | | | |

| | |
|---|---|
| p-value^{e} | 0.025 |

Note:
Includes only subjects with modified Mayo score 5-9 at induction baseline.
^{a}The confidence intervals for the proportion of subjects meeting the endpoint in each treatment group were based on the normal approximation confidence limits.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint were considered not to have a stool frequency subscore of 0 or 1 at the designated timepoint. For subjects who discontinued study agent due to COVID-19 related reasons (excluding COVID-19 infection) or regional crisis in Russia and Ukraine (ICE 4) prior to the designated timepoint, their observed values were used, if available. Subjects who experienced ICE 5 (discontinued study agent due to reasons other than those in ICEs 3 and 4) prior to the designated timepoint were considered not to have a stool frequency subscore of 0 or 1 at the designated timepoint.
^{c}Nonresponder Imputation for Missing Data: After accounting for ICEs, subjects who were missing stool frequency subscore at the designated timepoint were considered not to have a stool frequency subscore of 0 or 1.
^{d}The adjusted treatment difference and confidence intervals were based on the Wald statistic with Cochran-Mantel-Haenszel weight.
^{e}The p-values were based on the Cochran-Mantel-Haenszel (CMH) chi-square test, stratified by ADT-failure status (Yes/No) and concomitant use of corticosteroids at baseline (Yes/No).

TABLE 22
Number of Subjects With a Rectal Bleeding Subscore of 0 at Week
I-1; Full Analysis Set (Study CNTO1959UCO3001; Induction 2)

| | Placebo IV | Guselkumab 200 mg IV | |
|---|---|---|---|
| Analysis set: Full | 280 | 421 | |
| Week I-1 | | | |
| N | 280 | 421 | |
| Subjects with a rectal | 38 (13.6%) | 74 | (17.6%) |
| bleeding subscore of 0^{a,b} | | | |
| 95% CI for treatment | (9.6%, 17.6%) | (13.9%, 21.2%) | |
| proportion^{c} | | | |
| Adjusted treatment | | 4.1% | (−1.3%, 9.5%) |
| difference (95% CI)^{d} | | | |

| | |
|---|---|
| p-value^{e} | 0.148 |

Note:
Includes only subjects with modified Mayo score 5-9 at induction baseline.
^{a}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint were considered not to have a rectal bleeding subscore of 0 at the designated timepoint. For subjects who discontinued study agent due to COVID-19 related reasons (excluding COVID-19 infection) or regional crisis in Russia and Ukraine (ICE 4) prior to the designated timepoint, their observed values were used, if available. Subjects who experienced ICE 5 (discontinued study agent due to reasons other than those in ICEs 3 and 4) prior to the designated timepoint were considered not to have a rectal bleeding subscore of 0 at the designated timepoint.
^{b}Nonresponder Imputation for Missing Data: After accounting for ICEs, subjects who were missing rectal bleeding subscore at the designated timepoint were considered not to have a rectal bleeding subscore of 0.
^{c}The confidence intervals for the proportion of subjects meeting the endpoint in each treatment group were based on the normal approximation confidence limits.
^{d}The adjusted treatment difference and confidence intervals were based on the Wald statistic with Cochran-Mantel-Haenszel weight.
^{e}The p-values were based on the Cochran-Mantel-Haenszel (CMH) chi-square test, stratified by ADT-failure status (Yes/No) and concomitant use of corticosteroids at baseline (Yes/No).

TABLE 23
Summary of Change From Induction Baseline in Absolute
Stool Number from Week I-1 through Week I-12; Full
Analysis Set (Study CNTO1959UCO3001; Induction 2)

| | Placebo IV | | Guselkumab 200 mg IV | |
|---|---|---|---|---|
| Analysis set: Full | 280 | | 421 | |
| Absolute stool number | | | | |
| Baseline | | | | |
| N | 280 | | 421 | |
| Mean (SD) | 6.96 | (3.691) | 7.10 | (3.541) |
| Median | 6.00 | | 6.30 | |
| Range | (2.0; 30.0) | | (1.0; 27.3) | |
| IQ range | (4.70; 7.70) | | (4.70; 8.70) | |
| 95% CI^{a} | (6.5, 7.4) | | (6.8, 7.4) | |
| Week I-1^{b} | | | | |
| N | 254 | | 403 | |
| Mean (SD) | 6.55 | (3.604) | 6.27 | (3.484) |
| Median | 5.70 | | 5.70 | |
| Range | (0.7; 27.3) | | (0.7; 20.7) | |
| IQ range | (4.70; 7.70) | | (4.00; 7.70) | |
| 95% CI^{a} | (6.1, 7.0) | | (5.9, 6.6) | |
| Week I-2^{b} | | | | |
| N | 257 | | 406 | |
| Mean (SD) | 6.28 | (3.562) | 5.90 | (3.484) |
| Median | 5.30 | | 5.30 | |
| Range | (0.7; 24.7) | | (1.0; 27.3) | |
| IQ range | (4.00; 7.70) | | (3.70; 7.00) | |
| 95% CI^{a} | (5.8, 6.7) | | (5.6, 6.2) | |
| Week I-4^{b} | | | | |
| N | 277 | | 417 | |
| Mean (SD) | 6.11 | (3.830) | 5.05 | (3.222) |
| Median | 5.30 | | 4.30 | |
| Range | (0.3; 28.7) | | (0.3; 27.3) | |
| IQ range | (3.70; 7.30) | | (3.00; 6.30) | |
| 95% CI^{a} | (5.7, 6.6) | | (4.7, 5.4) | |
| Week I-8^{b} | | | | |
| N | 274 | | 419 | |
| Mean (SD) | 5.66 | (3.947) | 4.36 | (3.163) |
| Median | 5.00 | | 3.70 | |
| Range | (0.3; 30.0) | | (0.3; 27.3) | |
| IQ range | (3.30; 7.00) | | (2.30; 5.70) | |
| 95% CI^{a} | (5.2, 6.1) | | (4.1, 4.7) | |
| Week I-12^{b} | | | | |
| N | 274 | | 420 | |
| Mean (SD) | 5.62 | (3.907) | 3.95 | (3.207) |
| Median | 5.00 | | 3.00 | |
| Range | (0.7; 30.0) | | (0.0; 27.3) | |
| IQ range | (3.00; 7.00) | | (1.70; 5.00) | |
| 95% CI^{a} | (5.2, 6.1) | | (3.6, 4.3) | |
| Change from baseline | | | | |
| Week I-1^{b} | | | | |
| N | 254 | | 403 | |
| Mean (SD) | −0.37 | (2.220) | −0.77 | (2.090) |
| Median | −0.35 | | −0.70 | |
| Range | (−8.0; 6.4) | | (−8.0; 8.0) | |
| IQ range | (−1.40; 0.70) | | (−2.00; 0.30) | |
| 95% CI^{a} | (−0.6, −0.1) | | (−1.0, −0.6) | |
| Treatment difference | | | −0.38 | (−0.70, −0.06) |
| in LS Means (95% CI)^{c} | | | | |
| p-value^{d} | | | 0.021 | |
| Week I-2^{b} | | | | |
| N | 257 | | 406 | |
| Mean (SD) | −0.61 | (2.135) | −1.19 | (2.533) |
| Median | −0.60 | | −1.00 | |
| Range | (−9.0; 9.0) | | (−13.7; 11.0) | |
| IQ range | (−1.60; 0.60) | | (−2.40; 0.00) | |
| 95% CI^{a} | (−0.9, −0.3) | | (−1.4, −0.9) | |
| Treatment difference | | | −0.55 | (−0.89, −0.21) |
| in LS Means (95% CI)^{c} | | | | |
| p-value^{d} | | | 0.002 | |
| Week I-4^{b} | | | | |
| N | 277 | | 417 | |
| Mean (SD) | −0.75 | (2.792) | −2.05 | (2.728) |
| Median | −0.40 | | −1.70 | |
| Range | (−11.0; 9.4) | | (−14.4; 7.3) | |
| IQ range | (−2.00; 0.30) | | (−3.30; −0.30) | |
| 95% CI^{a} | (−1.1, −0.4) | | (−2.3, −1.8) | |
| Treatment difference | | | −1.25 | (−1.63, −0.87) |
| in LS Means (95% CI)^{c} | | | | |
| p-value^{d} | | | <0.001 | |
| Week I-8^{b} | | | | |
| N | 274 | | 419 | |
| Mean (SD) | −1.31 | (2.896) | −2.75 | (2.931) |
| Median | −1.00 | | −2.40 | |
| Range | (−16.3; 9.7) | | (−16.6; 6.7) | |
| IQ range | (−2.70; 0.00) | | (−4.30; −0.70) | |
| 95% CI^{a} | (−1.6, −1.0) | | (−3.0, −2.5) | |
| Treatment difference | | | −1.45 | (−1.85, −1.05) |
| in LS Means (95% CI)^{c} | | | | |
| p-value^{d} | | | <0.001 | |
| Week I-12^{b} | | | | |
| N | 274 | | 420 | |
| Mean (SD) | −1.36 | (3.011) | −3.15 | (3.189) |
| Median | −0.65 | | −3.00 | |
| Range | (−16.3; 7.7) | | (−16.0; 7.0) | |
| IQ range | (−2.70; 0.00) | | (−4.85; −0.70) | |
| 95% CI^{a} | (−1.7, −1.0) | | (−3.5, −2.8) | |
| Treatment difference | | | −1.79 | (−2.21, −1.37) |
| in LS Means (95% CI)^{c} | | | | |

| | |
|---|---|
| p-value^{d} | <0.001 |

Key: CI = confidence interval; IQ = interquartile; SD = standard deviation.
Note:
Includes only subjects with modified Mayo score 5-9 at induction baseline.
^{a}The confidence intervals for the mean in each treatment group were based on the normal approximation confidence limits.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint had their baseline value carried forward from the time of the event onward. For subjects who discontinued study agent due to COVID-19 related reasons (excluding COVID-19 infection) or regional crisis in Russia and Ukraine (ICE 4) prior to the designated timepoint, their observed values were used, if available. Subjects who experienced ICE 5 (discontinued study agent due to reasons other than those in ICEs 3 and 4) prior to the designated timepoint had their baseline value carried forward from the time of the event onward.
^{c}A Mixed-Effect Model Repeated Measures (MMRM) was used to account for missing data under the assumption of missing at random (MAR). In the MMRM, missing data was accounted for through correlation of repeated measures in the model. The treatment difference between guselkumab group and placebo was estimated by the difference in the least squares means (LS Means).
^{d}The p-values of treatment comparison were based on the MMRM.

TABLE 24
Summary of Change From Induction Baseline in Stool Frequency
and Rectal Bleeding Subscores from Week I-1 through Week I-
12; Full Analysis Set (Study CNTO1959UCO3001; Induction 2)

| | Placebo IV | | Guselkumab 200 mg IV | |
|---|---|---|---|---|
| Analysis set: Full | 280 | | 421 | |
| Stool frequency score | | | | |
| Baseline | | | | |
| N | 280 | | 421 | |
| Mean (SD) | 2.5 | (0.67) | 2.5 | (0.68) |
| Median | 3.0 | | 3.0 | |
| Range | (1; 3) | | (0; 3) | |
| IQ range | (2.0; 3.0) | | (2.0; 3.0) | |
| 95% CI^{a} | (2.4, 2.5) | | (2.4, 2.5) | |
| Week I-1^{b} | | | | |
| N | 254 | | 403 | |
| Mean (SD) | 2.3 | (0.82) | 2.2 | (0.89) |
| Median | 3.0 | | 2.0 | |
| Range | (0; 3) | | (0; 3) | |
| IQ range | (2.0; 3.0) | | (2.0; 3.0) | |
| 95% CI^{a} | (2.2, 2.4) | | (2.1, 2.3) | |
| Week I-2^{b} | | | | |
| N | 257 | | 406 | |
| Mean (SD) | 2.2 | (0.86) | 2.1 | (0.92) |
| Median | 2.0 | | 2.0 | |
| Range | (0; 3) | | (0; 3) | |
| IQ range | (2.0; 3.0) | | (1.0; 3.0) | |
| 95% CI^{a} | (2.1, 2.3) | | (2.0, 2.2) | |
| Week I-4^{b} | | | | |
| N | 277 | | 417 | |
| Mean (SD) | 2.1 | (0.93) | 1.8 | (0.98) |
| Median | 2.0 | | 2.0 | |
| Range | (0; 3) | | (0; 3) | |
| IQ range | (1.0; 3.0) | | (1.0; 3.0) | |
| 95% CI^{a} | (2.0, 2.2) | | (1.7, 1.9) | |
| Week I-8^{b} | | | | |
| N | 274 | | 419 | |
| Mean (SD) | 2.0 | (0.95) | 1.5 | (1.01) |
| Median | 2.0 | | 1.0 | |
| Range | (0; 3) | | (0; 3) | |
| IQ range | (1.0; 3.0) | | (1.0; 2.0) | |
| 95% CI^{a} | (1.9, 2.1) | | (1.4, 1.6) | |
| Week I-12^{b} | | | | |
| N | 274 | | 420 | |
| Mean (SD) | 2.0 | (1.03) | 1.4 | (1.04) |
| Median | 2.0 | | 1.0 | |
| Range | (0; 3) | | (0; 3) | |
| IQ range | (1.0; 3.0) | | (1.0; 2.0) | |
| 95% CI^{a} | (1.9, 2.1) | | (1.3, 1.5) | |
| Change from baseline | | | | |
| Week I-1^{b} | | | | |
| N | 254 | | 403 | |
| Mean (SD) | −0.2 | (0.73) | −0.3 | (0.72) |
| Median | 0.0 | | 0.0 | |
| Range | (−3; 2) | | (−3; 2) | |
| IQ range | (−1.0; 0.0) | | (−1.0; 0.0) | |
| 95% CI^{a} | (−0.3, −0.1) | | (−0.3, −0.2) | |
| Treatment difference | | | −0.1 | (−0.2, 0.0) |
| in LS Means (95% CI)^{c} | | | | |
| p-value^{d} | | | 0.064 | |
| Week I-2^{b} | | | | |
| N | 257 | | 406 | |
| Mean (SD) | −0.2 | (0.78) | −0.4 | (0.76) |
| Median | 0.0 | | 0.0 | |
| Range | (−3; 2) | | (−3; 1) | |
| IQ range | (−1.0; 0.0) | | (−1.0; 0.0) | |
| 95% CI^{a} | (−0.3, −0.2) | | (−0.5, −0.3) | |
| Treatment difference | | | −0.1 | (−0.2, 0.0) |
| in LS Means (95% CI)^{c} | | | | |
| p-value^{d} | | | 0.021 | |
| Week I-4^{b} | | | | |
| N | 277 | | 417 | |
| Mean (SD) | −0.3 | (0.86) | −0.7 | (0.91) |
| Median | 0.0 | | −1.0 | |
| Range | (−3; 1) | | (−3; 2) | |
| IQ range | (−1.0; 0.0) | | (−1.0; 0.0) | |
| 95% CI^{a} | (−0.4, −0.2) | | (−0.8, −0.6) | |
| Treatment difference | | | −0.3 | (−0.5, −0.2) |
| in LS Means (95% CI)^{c} | | | | |
| p-value^{d} | | | <0.001 | |
| Week I-8^{b} | | | | |
| N | 274 | | 419 | |
| Mean (SD) | −0.5 | (0.91) | −0.9 | (0.95) |
| Median | 0.0 | | −1.0 | |
| Range | (−3; 2) | | (−3; 1) | |
| IQ range | (−1.0; 0.0) | | (−2.0; 0.0) | |
| 95% CI^{a} | (−0.6, −0.4) | | (−1.0, −0.8) | |
| Treatment difference | | | −0.5 | (−0.6, −0.3) |
| in LS Means (95% CI)^{c} | | | | |
| p-value^{d} | | | <0.001 | |
| Week I-12^{b} | | | | |
| N | 274 | | 420 | |
| Mean (SD) | −0.5 | (1.00) | −1.1 | (1.05) |
| Median | 0.0 | | −1.0 | |
| Range | (−3; 2) | | (−3; 1) | |
| IQ range | (−1.0; 0.0) | | (−2.0; 0.0) | |
| 95% CI^{a} | (−0.6, −0.4) | | (−1.2, −1.0) | |
| Treatment difference | | | −0.6 | (−0.8, −0.5) |
| in LS Means (95% CI)^{c} | | | | |
| p-value^{d} | | | <0.001 | |
| Rectal bleeding score | | | | |
| Baseline | | | | |
| N | 280 | | 421 | |
| Mean (SD) | 1.8 | (0.65) | 1.7 | (0.67) |
| Median | 2.0 | | 2.0 | |
| Range | (1; 3) | | (0; 3) | |
| IQ range | (1.0; 2.0) | | (1.0; 2.0) | |
| 95% CI^{a} | (1.7, 1.8) | | (1.7, 1.8) | |
| Week I-1^{b} | | | | |
| N | 254 | | 403 | |
| Mean (SD) | 1.4 | (0.84) | 1.3 | (0.82) |
| Median | 1.0 | | 1.0 | |
| Range | (0; 3) | | (0; 3) | |
| IQ range | (1.0; 2.0) | | (1.0; 2.0) | |
| 95% CI^{a} | (1.3, 1.5) | | (1.2, 1.4) | |
| Week I-2^{b} | | | | |
| N | 257 | | 406 | |
| Mean (SD) | 1.3 | (0.90) | 1.1 | (0.84) |
| Median | 1.0 | | 1.0 | |
| Range | (0; 3) | | (0; 3) | |
| IQ range | (1.0; 2.0) | | (0.0; 2.0) | |
| 95% CI^{a} | (1.2, 1.4) | | (1.0, 1.2) | |
| Week I-4^{b} | | | | |
| N | 277 | | 417 | |
| Mean (SD) | 1.3 | (0.91) | 0.9 | (0.83) |
| Mediar | 1.0 | | 1.0 | |
| Range | (0; 3) | | (0; 3) | |
| IQ range | (1.0; 2.0) | | (0.0; 1.0) | |
| 95% CI^{a} | (1.2, 1.4) | | (0.8, 1.0) | |
| Week I-8^{b} | | | | |
| N | 274 | | 419 | |
| Mean (SD) | 1.1 | (0.97) | 0.6 | (0.81) |
| Median | 1.0 | | 0.0 | |
| Range | (0; 3) | | (0; 3) | |
| IQ range | (0.0; 2.0) | | (0.0; 1.0) | |
| 95% CI^{a} | (1.0, 1.2) | | (0.5, 0.7) | |
| Week I-12^{b} | | | | |
| N | 274 | | 420 | |
| Mean (SD) | 1.2 | (0.96) | 0.5 | (0.80) |
| Median | 1.0 | | 0.0 | |
| Range | (0; 3) | | (0; 3) | |
| IQ range | (0.0; 2.0) | | (0.0; 1.0) | |
| 95% CI^{a} | (1.1, 1.3) | | (0.4, 0.6) | |
| Change from baseline | | | | |
| Week I-1^{b} | | | | |
| N | 254 | | 403 | |
| Mean (SD) | −0.3 | (0.79) | −0.4 | (0.74) |
| Median | 0.0 | | 0.0 | |
| Range | (−3; 2) | | (−3; 1) | |
| IQ range | (−1.0; 0.0) | | (−1.0; 0.0) | |
| 95% CI^{a} | (−0.4, −0.2) | | (−0.5, −0.4) | |
| Treatment difference | | | −0.1 | (−0.3, 0.0) |
| in LS Means (95% CI)^{c} | | | | |
| p-value^{d} | | | 0.018 | |
| Week I-2^{b} | | | | |
| N | 257 | | 406 | |
| Mean (SD) | −0.5 | (0.86) | −0.6 | (0.82) |
| Median | 0.0 | | −1.0 | |
| Range | (−3; 1) | | (−3; 1) | |
| IQ range | (−1.0; 0.0) | | (−1.0; 0.0) | |
| 95% CI^{a} | (−0.6, −0.3) | | (−0.7, −0.5) | |
| Treatment difference | | | −0.2 | (−0.3, −0.1) |
| in LS Means (95% CI)^{c} | | | | |
| p-value^{d} | | | 0.003 | |
| Week I-4^{b} | | | | |
| N | 277 | | 417 | |
| Mean (SD) | −0.5 | (0.92) | −0.8 | (0.88) |
| Median | 0.0 | | −1.0 | |
| Range | (−3; 2) | | (−3; 2) | |
| IQ range | (−1.0; 0.0) | | (−1.0; 0.0) | |
| 95% CI^{a} | (−0.6, −0.4) | | (−0.9, −0.8) | |
| Treatment difference | | | −0.4 | (−0.5, −0.2) |
| in LS Means (95% CI)^{c} | | | | |
| p-value^{d} | | | <0.001 | |
| Week I-8^{b} | | | | |
| N | 274 | | 419 | |
| Mean (SD) | −0.6 | (0.98) | −1.1 | (0.92) |
| Median | 0.0 | | −1.0 | |
| Range | (−3; 2) | | (−3; 2) | |
| IQ range | (−1.0; 0.0) | | (−2.0; 0.0) | |
| 95% CI^{a} | (−0.8, −0.5) | | (−1.2, −1.0) | |
| Treatment difference | | | −0.5 | (−0.6, −0.4) |
| in LS Means (95% CI)^{c} | | | | |
| p-value^{d} | | | <0.001 | |
| Week I-12^{b} | | | | |
| N | 274 | | 420 | |
| Mean (SD) | −0.6 | (0.91) | −1.2 | (0.95) |
| Median | 0.0 | | −1.0 | |
| Range | (−3; 1) | | (−3; 1) | |
| IQ range | (−1.0; 0.0) | | (−2.0; 0.0) | |
| 95% CI^{a} | (−0.7, −0.5) | | (−1.3, −1.1) | |
| Treatment difference | | | −0.7 | (−0.8, −0.5) |
| in LS Means (95% CI)^{c} | | | | |

| | |
|---|---|
| p-value^{d} | <0.001 |

Key: CI = confidence interval; IQ = interquartile; SD = standard deviation.
Note:
Includes only subjects with modified Mayo score 5-9 at induction baseline.
^{a}The confidence intervals for the mean in each treatment group were based on the normal approximation confidence limits.
^{b}Intercurrent Event (ICE) Strategies: Subjects who had an ostomy or colectomy (ICE 1), a prohibited change in UC medications (ICE 2), or discontinued study agent due to lack of efficacy or an AE of worsening of UC (ICE 3) prior to the designated timepoint had their baseline value carried forward from the time of the event onward. For subjects who discontinued study agent due to COVID-19 related reasons (excluding COVID-19 infection) or regional crisis in Russia and Ukraine (ICE 4) prior to the designated timepoint, their observed values were used, if available. Subjects who experienced ICE 5 (discontinued study agent due to reasons other than those in ICEs 3 and 4) prior to the designated timepoint had their baseline value carried forward from12832 the time of the event onward.
^{c}A Mixed-Effect Model Repeated Measures (MMRM) was used to account for missing data under the assumption of missing at random (MAR). In the MMRM, missing data was accounted for through correlation of repeated measures in the model. The treatment difference between guselkumab group and placebo was estimated by the difference in the least squares means (LS Means).
^{d}The p-values of treatment comparison were based on the MMRM.

TABLE 25
Number of Subjects With Symptomatic Mayo Score Improvement Through
Week I-12; Full Analysis Set (Study CNTO1959UCO3001; Induction 1)

| | | Guselkumab | | | |
|---|---|---|---|---|---|
| | Placebo IV | 200 mg IV | 400 mg IV | Combined | |
| Analysis set: Full | 105 | 101 | 107 | 208 | |
| Week I-2 | | | | | |
| N | 105 | 101 | 107 | 208 | |
| Subjects with | 25 (23.8%) | 41 (40.6%) | 40 (37.4%) | 81 | (38.9%) |
| symptomatic Mayo | | | | | |
| score improvement^{a,b,c,d} | | | | | |
| 95% CI for treatment | (15.7%, 32.0%) | (31.0%, 50.2%) | (28.2%, 46.6%) | (32.3%, | |
| proportion^{e} | | | | 45.6%) | |
| Adjusted treatment | | 16.9 (4.4, | 13.5 (1.5, | 15.1 (4.7, | |
| difference (95% CI)^{f} | | 29.4) | 25.6) | 25.5) | |
| p-value^{g} | | 0.010 | 0.033 | 0.008 | |
| Week I-4 | | | | | |
| N | 105 | 101 | 107 | 208 | |
| Subjects with | 25 (23.8%) | 63 (62.4%) | 54 (50.5%) | 117 | (56.3%) |
| symptomatic Mayo | | | | | |
| score improvement^{a,b,c,d} | | | | | |
| 95% CI for treatment | (15.7%, 32.0%) | (52.9%, 71.8%) | (41.0%, 59.9%) | (49.5%, 63.0%) | |
| proportione | | | | | |
| Adjusted treatment | | 38.2 (26.0, | 26.7 (14.5, | 32.3 (21.9, | |
| difference (95% CI)^{f} | | 50.4) | 38.8) | 42.7) | |
| p-value^{g} | | <0.001 | <0.001 | <0.001 | |
| Week I-8 | | | | | |
| N | 105 | 101 | 107 | 208 | |
| Subjects with | 40 (38.1%) | 66 (65.3%) | 79 (73.8%) | 145 | (69.7%) |
| symptomatic Mayo | | | | | |
| score improvement^{a,b,c,d} | | | | | |
| 95% CI for treatment | (28.8%, 47.4%) | (56.1%, 74.6%) | (65.5%, 82.2%) | (63.5%, 76.0%) | |
| proportion^{e} | | | | | |
| Adjusted treatment | | 27.0 (14.2, | 35.8 (23.7, | 31.6 (20.6, | |
| difference (95% CI)^{f} | | 39.9) | 47.8) | 42.5) | |
| p-value^{g} | | <0.001 | <0.001 | <0.001 | |
| Week I-12 | | | | | |
| N | 105 | 101 | 107 | 208 | |
| Subjects with | 39 (37.1%) | 66 (65.3%) | 71 (66.4%) | 137 | (65.9%) |

| symptomatic Mayo | | | | |
|---|---|---|---|---|
| score improvement^{a,b,c,d} | | | | |
| 95% CI for treatment | (27.9%, 46.4%) | (56.1%, 74.6%) | (57.4%, 75.3%) | (59.4%, 72.3%) |
| proportion^{e} | | | | |
| Adjusted treatment | | 28.0 (15.1, | 29.2 (16.5, | 28.6 (17.5, |
| difference (95% CI)^{f} | | 40.9) | 41.8) | 39.7) |
| p-value^{g} | | <0.001 | <0.001 | <0.001 |

Note:
Includes only subjects with modified Mayo score 5-9 at induction baseline.
^{a}Symptomatic Mayo score improvement is defined as a decrease from induction baseline in the symptomatic Mayo score by ≥30% and ≥1 point, with either a ≥1 point decrease from baseline in the rectal bleeding subscore or a rectal bleeding subscore of 0 or 1. The symptomatic Mayo score is defined as the sum of the stool frequency and the rectal bleeding subscores.
^{b}Subjects who had a prohibited change in UC medication, an ostomy or colectomy, or discontinued study agent due to lack of efficacy or an AE of worsening of UC prior to the designated timepoint were considered not to have achieved symptomatic Mayo score improvement.
^{c}Data after a discontinuation of study agent due to COVID-19 related reasons (excluding COVID-19 infection) were considered to be missing.
^{d}Subjects who were missing one or more Mayo subscore pertaining to this endpoint (stool frequency and/or rectal bleeding) at the designated timepoint were considered not to have achieved symptomatic Mayo score improvement.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) chi-square test.

TABLE 26
Primary Endpoint Analysis (Primary Estimand): Number of Subjects in Clinical Remission
at Week M-44; Randomized Full Analysis Set (Study CNTO1959UCO3001; Maintenance)

| | | Guselkumab | | |
|---|---|---|---|---|
| | Placebo SC^{a} | 100 mg SC q8w | 200 mg SC q4w | Combined |
| Analysis set: Randomized Full | 190 | 188 | 190 | 378 |
| _Week M-44 | | | | |
| N | 190 | 188 | 190 | 378 |
| Subjects in clinical remission^{b,c,d} | 36 (18.9%) | 85 (45.2%) | 95 (50.0%) | 180 (47.6%) |
| 95% CI for treatment proportion^{e} | (13.4%, 24.5%) | (38.1%, 52.3%) | (42.9%, 57.1%) | (42.6%, 52.7%) |
| Adjusted treatment difference (95% CI)^{f} | | 25.2% (16.4%, | 29.5% (20.9%, | 27.4% (20.2%, |
| | | 33.9%) | 38.1%) | 34.6%) |
| p-value^{g} | | <0.001 | <0.001 | <0.001 |

Note:
Includes only subjects with modified Mayo score 5-9 at induction baseline.
^{a}Subjects who were in clinical response to guselkumab IV induction dosing and were randomized to placebo SC on entry into this maintenance study.
^{b}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{c}Intercurrent Event (ICE) Strategies: Subjects who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (ICE 4) prior to Week M-44 were considered not to be in clinical remission at Week M-44. For subjects who discontinued study agent due to COVID-19 related reasons (excluding COVID-19 infection) or regional crisis in Russia and Ukraine (ICE 5) prior to Week M-44, their observed values (if available) were used. Subjects who experienced ICE 6 (discontinued study agent due to reasons other than those in ICEs 4 and 5) prior to Week M-44 were considered not to be in clinical remission at Week M-44.
^{d}Nonresponder Imputation for Missing Data: After accounting for ICEs, subjects who were missing one or more Mayo subscore pertaining to this endpoint (stool frequency, rectal bleeding, or endoscopy) at Week M-44 were considered not to be in clinical remission.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) test, stratified by clinical remission status at maintenance baseline (Yes/No), and induction treatment (guselkumab 400 mg IV, guselkumab 200 mg IV, placebo IV crossover to guselkumab 200 mg IV).

TABLE 27
Summary of Major Secondary Endpoints at Week M-44; Randomized
Full Analysis Set (Ordered by Global Testing Procedure)

| | Placebo SC | | GUS 100 mg q8w SC | GUS 200 mg q4w SC |
|---|---|---|---|---|
| Endpoints | (N = 190) | | (N = 188) | (N = 190) |
| Symptomatic remission | 71 | (37.4%) | 132 (70.2%) | 131 (68.9)% |
| | | | 31.9% (22.5%, 41.2%)^{a,}*** | 30.5% (21.2%, 39.9%)^{a,}*** |
| Endoscopic healing | 36 | (18.9%) | 93 (49.5%) | 98 (51.6%) |
| | | | 29.5% (20.7%, 38.3%)^{a,}*** | 31.1% (22.5%, 39.8%)^{a,}*** |
| Corticosteroid-free | 35 | (18.4%) | 85 (45.2%) | 93 (48.9%) |
| clinical remission | | | 25.7% (17.0%, 34.5%)^{a,}*** | 29.0% (20.5%, 37.6%)^{a,}*** |
| Clinical response | 82 | (43.2%) | 146 (77.7%) | 142 (74.7%) |
| | | | 33.6% (24.5%, 42.7%)^{a,}*** | 30.7% (21.5%, 40.0%)^{a,}*** |
| Histologic-endoscopic | 32 | (16.8%) | 82 (43.6%) | 91 (47.9%) |
| mucosal healing | | | 25.7% (17.1%, 34.3%)^{a,}*** | 29.6% (21.1%, 38.0%)^{a,}*** |
| IBDQ remission | 71 | (37.4%) | 121 (64.4%) | 122 (64.2%) |
| | | | 26.3% (16.8%, 35.7%)^{a,}*** | 25.9% (16.5%, 35.4%)^{a,}*** |
| Fatigue response | 56 | (29.5%) | 95 (50.5%) | 82 (43.2%) |
| | | | 20.1% (10.7%, 29.5%)^{a,}*** | 12.6% (3.3%, 21.9%)^{a,}** |
| Maintenance of clinical | 20/59 | (33.9%) | 40/66 (60.6%) | 50/69 (72.5%) |
| remission^{b} | | | 25.9% (9.2%, 42.5%)^{a,}** | 38.4% (22.7%, 54.1%)^{a,}*** |
| Endoscopic | 29 | (15.3%) | 65 (34.6%) | 64 (33.7%) |
| normalization | | | 18.5% (10.2%, 26.8%)^{a,}*** | 16.8% (9.0%, 24.7%)^{a,}*** |

* p < 0.05;
**p < 0.01;
***p < 0.001. The p-values were based on the Cochran-Mantel-Haenszel test (Appendix 2). Significance of the results based on the US-specific testing procedure is shown in Attachment 4.
^{a}Adjusted treatment difference (95% CI).
^{b}Denominator is the number of subjects in clinical remission at maintenance baseline.

TABLE 28
Summary of Key Efficacy Major Secondary Endpoints Analysis (Main Estimand) at
Week M-44; Randomized Full Analysis Set (Study CNTO1959UCO3001; Maintenance)

| | | Guselkumab | | | | | |
|---|---|---|---|---|---|---|---|
| | Placebo SC^{a} | 100 mg SC q8w | | 200 mg SC q4w | | Combined | |
| Analysis set: Randomized Full | 190 | 188 | | 190 | | 378 | |
| Subjects in symptomatic remission at Week M- | 71 (37.4%) | 132 | (70.2%) | 131 | (68.9%) | 263 | (69.6%) |
| 44^{b1,c,d} | | | | | | | |
| 95% CI for treatment proportion^{e} | (30.5%, 44.2%) | (63.7%, 76.7%) | | (62.4%, 75.5%) | | (64.9%, 74.2%) | |
| Adjusted treatment difference (95% CI)^{f} | | 31.9% (22.5%, | | 30.5% (21.2%, | | 31.3% (23.1%, | |
| | | 41.2%) | | 39.9%) | | 39.5%) | |
| p-value^{g} | | <0.001 | | <0.001 | | <0.001 | |
| Subjects with endoscopic healing at Week M- | 36 (18.9%) | 93 | (49.5%) | 98 | (51.6%) | 191 | (50.5%) |
| 44^{b2,c,d} | | | | | | | |
| 95% CI for treatment proportion^{e} | (13.4%, 24.5%) | (42.3%, 56.6%) | | (44.5%, 58.7%) | | (45.5%, 55.6%) | |
| Adjusted treatment difference (95% CI)^{f} | | 29.5% (20.7%, | | 31.1% (22.5%, | | 30.4% (23.1%, | |
| | | 38.3%) | | 39.8%) | | 37.6%) | |
| p-value^{g} | | <0.001 | | <0.001 | | <0.001 | |
| Subjects in corticosteroid-free clinical remission | 35 (18.4%) | 85 | (45.2%) | 93 | (48.9%) | 178 | (47.1%) |
| at Week M-44^{b3,c,d} | | | | | | | |
| 95% CI for treatment proportion^{e} | (12.9%, 23.9%) | (38.1%, 52.3%) | | (41.8%, 56.1%) | | (42.1%, 52.1%) | |
| Adjusted treatment difference (95% CI)^{f} | | 25.7% (17.0%, | | 29.0% (20.5%, | | 27.4% (20.3%, | |
| | | 34.5%) | | 37.6%) | | 34.6%) | |
| p-value^{g} | | <0.001 | | <0.001 | | <0.001 | |
| Subjects in clinical response at Week M-44^{b4,c,d} | 82 (43.2%) | 146 | (77.7%) | 142 | (74.7%) | 288 | (76.2%) |
| 95% CI for treatment proportion^{e} | (36.1%, 50.2%) | (71.7%, 83.6%) | | (68.6%, 80.9%) | | (71.9%, 80.5%) | |
| Adjusted treatment difference (95% CI)^{f} | | 33.6% (24.5%, | | 30.7% (21.5%, | | 32.3% (24.2%, | |
| | | 42.7%) | | 40.0%) | | 40.5%) | |
| p-value^{g} | | <0.001 | | <0.001 | | <0.001 | |
| Subjects with histologic-endoscopic mucosal | 32 (16.8%) | 82 | (43.6%) | 91 | (47.9%) | 173 | (45.8%) |
| healing at Week M-44^{b5,c,d} | | | | | | | |
| 95% CI for treatment proportion^{e} | (11.5%, 22.2%) | (36.5%, 50.7%) | | (40.8%, 55.0%) | | (40.7%, 50.8%) | |
| Adjusted treatment difference (95% CI)^{f} | | 25.7% (17.1%, | | 29.6% (21.1%, | | 27.7% (20.6%, | |
| | | 34.3%) | | 38.0%) | | 34.7%) | |
| p-value^{g} | | <0.001 | | <0.001 | | <0.001 | |
| Subjects in IBDQ remission at Week M-44^{b6,c,d} | 71 (37.4%) | 121 | (64.4%) | 122 | (64.2%) | 243 | (64.3%) |
| 95% CI for treatment proportion^{e} | (30.5%, 44.2%) | (57.5%, 71.2%) | | (57.4%, 71.0%) | | (59.5%, 69.1%) | |
| Adjusted treatment difference (95% CI)^{f} | | 26.3% (16.8%, | | 25.9% (16.5%, | | 26.2% (18.0%, | |
| | | 35.7%) | | 35.4%) | | 34.5%) | |
| p-value^{g} | | <0.001 | | <0.001 | | <0.001 | |
| Subjects in fatigue response at Week M-44^{b7,c,d} | 56 (29.5%) | 95 | (50.5%) | 82 | (43.2%) | 177 | (46.8%) |
| 95% CI for treatment proportion^{e} | (23.0%, 36.0%) | (43.4%, 57.7%) | | (36.1%, 50.2%) | | (41.8%, 51.9%) | |
| Adjusted treatment difference (95% CI)^{f} | | 20.1% (10.7%, | | 12.6% (3.3%, | | 16.4% (8.3%, | |
| | | 29.5%) | | 21.9%) | | 24.4%) | |
| p-value^{g} | | <0.001 | | 0.009 | | <0.001 | |
| Subjects in clinical remission at maintenance | 59 (31.1%) | 66 | (35.1%) | 69 | (36.3%) | 135 | (35.7%) |
| baseline^{b8} | | | | | | | |
| Subjects in clinical remission at Week M- | 20 (33.9%) | 40 | (60.6%) | 50 | (72.5%) | 90 | (66.7%) |
| 44^{b8,c,d,h} | | | | | | | |
| 95% CI for treatment proportion^{e} | (21.8%, 46.0%) | (48.8%, 72.4%) | | (61.9%, 83.0%) | | (58.7%, 74.6%) | |
| Adjusted treatment difference (95% CI)^{f} | | 25.9% (9.2%, | | 38.4% (22.7%, | | 32.4% (18.3%, | |
| | | 42.5%) | | 54.1%) | | 46.6%) | |
| p-valueⁱ | | 0.004 | | <0.001 | | <0.001 | |
| Subjects with endoscopic normalization at Week | 29 (15.3%) | 65 | (34.6%) | 64 | (33.7%) | 129 | (34.1%) |

| M-44^{b9,c,d} | | | | |
|---|---|---|---|---|
| 95% CI for treatment proportion^{e} | (10.1%, 20.4%) | (27.8%, 41.4%) | (27.0%, 40.4%) | (29.3%, 38.9%) |
| Adjusted treatment difference (95% CI)^{f} | | 18.5% (10.2%, | 16.8% (9.0%, | 17.7% (11.0%, |
| | | 26.8%) | 24.7%) | 24.4%) |
| p-value^{g} | | <0.001 | <0.001 | <0.001 |

Note:
Includes only subjects with modified Mayo score 5-9 at induction baseline.
^{a}Subjects who were in clinical response to guselkumab IV induction dosing and were randomized to placebo SC on entry into this maintenance study.
^{b1}Symptomatic remission is defined as a stool frequency subscore of 0 or 1 and a rectal bleeding subscore of 0, where the stool frequency subscore has not increased from induction baseline.
^{b2}Endoscopic healing is defined as an endoscopy subscore of 0 or 1 with no friability present on the endoscopy.
^{b3}Corticosteroid-free clinical remission is defined as not requiring any treatment with corticosteroids for at least 8 weeks prior to Week M-44 and also meeting the criteria for clinical remission at Week M-44.
^{b4}Clinical response is defined as a decrease from induction baseline in the modified Mayo score by ≥30% and ≥2 points, with either a ≥1-point decrease from induction baseline in the rectal bleeding subscore or a rectal bleeding subscore of 0 or 1.
^{b5}Histologic-endoscopic mucosal healing is defined as achieving a combination of histologic healing and endoscopic healing. Histologic healing is defined as neutrophil infiltration in <5% of crypts, no crypt destruction, and no erosions, ulcerations or granulation tissue according to the Geboes grading system. Endoscopic healing is defined as an endoscopy subscore of 0 or 1 with no friability present on the endoscopy.
^{b6}IBDQ (Inflammatory Bowel Disease Questionnaire) remission is defined as a total IBDQ score ≥170.
^{b7}Fatigue response is defined as ≥7-point improvement from induction baseline in the PROMIS (Patient-Reported Outcomes Measurement Information System) Fatigue Short Form 7a.
^{b8}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{b9}Endoscopic normalization is defined as an endoscopy subscore of 0.
^{c}Intercurrent Event (ICE) Strategies: Subjects who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (TCE 4) prior to Week M-44 were considered not to have achieved any of the key efficacy endpoints shown at Week M-44. For subjects who discontinued study agent due to COVID-19 related reasons (excluding COVID-19 infection) or regional crisis in Russia and Ukraine (ICE 5) prior to Week M-44, their observed values (if available) were used. Subjects who experienced ICE 6 (discontinued study agent due to reasons other than those in ICEs 4 and 5) prior to Week M-44 were considered not to have achieved any of the key efficacy endpoints shown at Week M-44.
^{d}Nonresponder Imputation for Missing Data: Subjects who were missing one or more of the components pertaining to an endpoint at Week M-44 were considered not to have achieved the endpoint. Subjects who had an unevaluable biopsy (i.e., a biopsy that was collected, but could not be assessed due to sample preparation or technical errors) were considered not to have achieved the histology endpoints.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) test, stratified by clinical remission status at maintenance baseline (Yes/No), and induction treatment (guselkumab 400 mg IV, guselkumab 200 mg IV, placebo IV crossover to guselkumab 200 mg IV).
^{h}Denominator is based on the subjects in clinical remission at maintenance baseline.
ⁱThe p-values were based on the Cochran-Mantel-Haenszel (CMH) test, stratified by induction treatment (guselkumab 400 mg IV, guselkumab 200 mg IV, placebo IV crossover to guselkumab 200 mg IV).

TABLE 29
Summary of UC Disease Characteristics at Induction Baseline; Full Analysis Set (Study CNTO1959UCO3001; Maintenance)

| | | | | | | Nonrandomized Subjects | | |
|---|---|---|---|---|---|---|---|---|
| | Randomized Subjects | | | | | | Induction | |
| | Induction Guselkumab IV Responders | | | | | Induction | Guselkumab | |
| | | Guselkumab | | | | Placebo IV | 24-week Responders^{c} | |
| | Placebo | 100 mg | 200 mg | | | Responders | Guselkumab 200 | Overall |
| | SC^{a} | SC q8w | SC q4w | Combined | Total | Placebo SC^{b} | mg SC q4w | Total |
| Analysis set: Full | 190 | 188 | 190 | 378 | 568 | 114 | 123 | 805 |
| UC disease duration | | | | | | | | |
| (years) | | | | | | | | |
| N | 190 | 188 | 190 | 378 | 568 | 114 | 123 | 805 |
| Mean (SD) | 7.29 | 7.78 | 8.35 | 8.07 | 7.81 | 7.31 | 7.17 | 7.64 |
| | (6.338) | (8.463) | (8.397) | (8.424) | (7.791) | (6.273) | (5.428) | (7.272) |
| Median | 5.13 | 5.11 | 6.05 | 5.65 | 5.39 | 6.17 | 5.86 | 5.72 |
| Range | (0.3; 35.4) | (0.3; 47.7) | (0.4; 44.5) | (0.3; 47.7) | (0.3; 47.7) | (0.3; 41.1) | (0.7; 25.7) | (0.3; 47.7) |
| IQ range | (2.79; 10.50) | (2.00; 9.74) | (2.22; 11.14) | (2.20; 10.80) | (2.48; 10.75) | (2.41; 9.60) | (2.87; 9.42) | (2.56; 10.30) |
| Extent of disease | | | | | | | | |
| N | 190 | 188 | 190 | 378 | 568 | 114 | 123 | 805 |
| Limited to left | 95 | 109 | 107 | 216 | 311 | 55 | 64 | 430 |
| side of colon | (50.0%) | (58.0%) | (56.3%) | (57.1%) | (54.8%) | (48.2%) | (52.0%) | (53.4%) |
| Extensive | 95 | 79 | 83 | 162 | 257 | 59 | 59 | 375 |
| | (50.0%) | (42.0%) | (43.7%) | (42.9%) | (45.2%) | (51.8%) | (48.0%) | (46.6%) |
| Mayo score | | | | | | | | |
| N | 190 | 188 | 190 | 378 | 568 | 114 | 123 | 805 |
| Mean (SD) | 9.2 | 9.0 | 9.2 | 9.1 | 9.1 | 9.0 | 9.2 | 9.1 |
| | (1.37) | (1.39) | (1.38) | (1.39) | (1.38) | (1.36) | (1.18) | (1.35) |
| Median | 9.0 | 9.0 | 9.0 | 9.0 | 9.0 | 9.0 | 9.0 | 9.0 |
| Range | (6; 12) | (6; 12) | (6; 12) | (6; 12) | (6; 12) | (7; 12) | (7; 12) | (6; 12) |
| IQ range | (8.0; 10.0) | (8.0; 10.0) | (8.0; 10.0) | (8.0; 10.0) | (8.0; 10.0) | (8.0; 10.0) | (8.0; 10.0) | (8.0; 10.0) |
| Severity of UC | | | | | | | | |
| disease by Mayo | | | | | | | | |
| score | | | | | | | | |
| N | 190 | 188 | 190 | 378 | 568 | 114 | 123 | 805 |
| Moderate | 152 | 161 | 153 | 314 | 466 | 96 | 105 | 667 |
| (Mayo score 6-10) | (80.0%) | (85.6%) | (80.5%) | (83.1%) | (82.0%) | (84.2%) | (85.4%) | (82.9%) |
| Severe (Mayo | 38 | 27 | 37 | 64 | 102 | 18 | 18 | 138 |
| score 11-12) | (20.0%) | (14.4%) | (19.5%) | (16.9%) | (18.0%) | (15.8%) | (14.6%) | (17.1%) |
| Modified Mayo score | | | | | | | | |
| N | 190 | 188 | 190 | 378 | 568 | 114 | 123 | 805 |
| Mean (SD) | 7.0 | 6.8 | 6.9 | 6.9 | 6.9 | 6.8 | 7.0 | 6.9 |
| | (1.09) | (1.15) | (1.10) | (1.13) | (1.12) | (1.13) | (0.98) | (1.10) |
| Median Range | 7.0 | 7.0 | 7.0 | 7.0 | 7.0 | 7.0 | 7.0 | 7.0 |
| | (5; 9) | (5; 9) | (5; 9) | (5; 9) | (5; 9) | (5; 9) | (5; 9) | (5; 9) |
| IQ range | (6.0; 8.0) | (6.0; 8.0) | (6.0; 8.0) | (6.0; 8.0) | (6.0; 8.0) | (6.0; 8.0) | (6.0; 8.0) | (6.0; 8.0) |
| Severity of UC | | | | | | | | |
| disease by modified | | | | | | | | |
| Mayo score | | | | | | | | |
| N | 190 | 188 | 190 | 378 | 568 | 114 | 123 | 805 |
| Moderate | 65 | 74 | 66 | 140 | 205 | 47 | 31 | 283 |
| (modified | (34.2%) | (39.4%) | (34.7%) | (37.0%) | (36.1%) | (41.2%) | (25.2%) | (35.2%) |
| Mayo score 5-6) | | | | | | | | |
| Severe | 125 | 114 | 124 | 238 | 363 | 67 | 92 | 522 |
| (modified | (65.8%) | (60.6%) | (65.3%) | (63.0%) | (63.9%) | (58.8%) | (74.8%) | (64.8%) |
| Mayo score 7-9) | | | | | | | | |
| N | 190 | 188 | 190 | 378 | 568 | 114 | 123 | 805 |
| Mean (SD) | 6.5 | 6.3 | 6.5 | 6.4 | 6.5 | 6.4 | 6.4 | 6.4 |
| | (1.23) | (1.26) | (1.25) | (1.25) | (1.25) | (1.21) | (1.09) | (1.22) |
| Median | 7.0 | 6.0 | 6.0 | 6.0 | 6.0 | 6.0 | 6.0 | 6.0 |
| Range | (4; 9) | (4; 9) | (3; 9) | (3; 9) | (3; 9) | (4; 9) | (4; 9) | (3; 9) |
| IQ range | (6.0; 7.0) | (5.0; 7.0) | (6.0; 7.0) | (6.0; 7.0) | (6.0; 7.0) | (6.0; 7.0) | (6.0; 7.0) | (6.0; 7.0) |
| Severity of | | | | | | | | |
| endoscopy subscore | | | | | | | | |
| N | 190 | 188 | 190 | 378 | 568 | 114 | 123 | 805 |
| Moderate | 61 | 63 | 67 | 130 | 191 | 44 | 28 | 263 |
| (endoscopy | (32.1%) | (33.5%) | (35.3%) | (34.4%) | (33.6%) | (38.6%) | (22.8%) | (32.7%) |
| subscore = 2) | | | | | | | | |
| Severe | 129 | 125 | 123 | 248 | 377 | 70 | 95 | 542 |
| (endoscopy | (67.9%) | (66.5%) | (64.7%) | (65.6%) | (66.4%) | (61.4%) | (77.2%) | (67.3%) |
| subscore = 3) | | | | | | | | |
| Extraintestinal | | | | | | | | |
| manifestations | | | | | | | | |
| N | 190 | 188 | 190 | 378 | 568 | 114 | 123 | 805 |
| Present | 23 | 22 | 23 | 45 | 68 | 11 | 23 | 102 |
| | (12.1%) | (11.7%) | (12.1%) | (11.9%) | (12.0%) | (9.6%) | (18.7%) | (12.7%) |
| Absent | 167 | 166 | 167 | 333 (88.1%) | 500 | 103 (90.4%) | 100 (81.3%) | 703 (87.3%) |
| | (87.9%) | (88.3%) | (87.9%) | | (88.0%) | | | |
| CRP (mg/L) | | | | | | | | |
| N | 190 | 185 | 187 | 372 | 562 | 113 | 123 | 798 |
| Mean (SD) | 8.7 | 9.1 | 8.3 | 8.7 | 8.7 | 6.5 | 9.8 | 8.6 |
| | (12.52) | (14.23) | (13.61) | (13.91) | (13.45) | (7.93) | (11.79) | (12.58) |
| Median Range | 4.2 | 3.9 | 3.6 | 3.7 | 3.9 | 3.4 | 5.0 | 3.9 |
| | (0; 90) | (0; 98) | (0; 113) | (0; 113) | (0; 113) | (0; 43) | (0; 53) | (0; 113) |
| IQ range | (1.6; 8.4) | (1.4; | (1.4; 9.1) | (1.4; 9.7) | (1.5; 9.2) | (0.9; 8.2) | (1.6; 14.3) | (1.4; 9.6) |
| | | 10.4) | | | | | | |
| Abnormal CRP | 117 | 104 | 104 | 208 | 325 | 59 | 78 | 462 |
| (>3 mg/L) | (61.6%) | (56.2%) | (55.6%) | (55.9%) | (57.8%) | (52.2%) | (63.4%) | (57.9%) |
| Fecal calprotectin | | | | | | | | |
| (mg/kg) | | | | | | | | |
| N | 175 | 160 | 171 | 331 | 506 | 101 | 114 | 721 |
| Mean (SD) | 2991.8 | 3228.5 | 3082.4 | 3153.0 | 3097.3 | 2689.6 | 2838.2 | 2999.2 |
| | (4411.45) | (4730.23) | (4594.23) | (4653.96) | (4567.81) | (5243.48) | (3395.33) | (4504.07) |
| Median Range | 1642.0 | 1675.0 | 1487.0 | 1586.0 | 1605.0 | 1484.0 | 1720.5 | 1595.0 |
| | (15; 36000) | (15; 36000) | (15; 23654) | (15; 36000) | (15; 36000) | (15; 36000) | (123; 22483) | (15; 36000) |
| IQ range | (663.0; | (806.0; | (603.0; | (674.0; | (669.0; | (571.0; | (811.0; | (672.0; |
| | 3498.0) | 3543.5) | 3019.0) | 3261.0) | 3337.0) | 2160.0) | 3275.0) | 3241.0) |
| Abnormal fecal | 154 | 141 | 150 | 291 | 445 | 88 | 107 | 640 |
| calprotectin | (88.0%) | (88.1%) | (87.7%) | (87.9%) | (87.9%) | (87.1%) | (93.9%) | (88.8%) |
| (>250 mg/kg) | | | | | | | | |
| Fecal | 162 | 148 | 159 | 307 | 469 | 93 | 111 | 673 |
| calprotectin > | (92.6%) | (92.5%) | (93.0%) | (92.7%) | (92.7%) | (92.1%) | (97.4%) | (93.3%) |
| 150 mg/kg | | | | | | | | |
| Albumin (g/L) | | | | | | | | |
| N | 190 | 188 | 190 | 378 | 568 | 114 | 123 | 805 |
| Mean (SD) | 42.9 | 43.3 | 42.8 | 43.0 | 43.0 | 43.8 | 42.9 | 43.1 |
| | (4.20) | (4.29) | (4.10) | (4.20) | (4.20) | (3.60) | (3.72) | (4.05) |
| Median Range | 43.0 | 44.0 | 43.0 | 43.0 | 43.0 | 44.0 | 43.0 | 43.0 |
| | (27; 55) | (16; 54) | (31; 52) | (16; 54) | (16; 55) | (34; 53) | (28; 51) | (16; 55) |
| IQ range | (41.0; 46.0) | (41.0; 46.0) | (40.0; 46.0) | (41.0; 46.0) | (41.0; 46.0) | (42.0; 46.0) | (40.0; 45.0) | (41.0; 46.0) |

Key: CRP = C-reactive protein; IQ = interquartile; SD = standard deviation.
Note:
Includes only subjects with modified Mayo score 5-9 at induction baseline.
Note:
Subjects were presented in the treatment group assigned at Week M-0.
^{a}Subjects who were in clinical response to guselkumab IV induction dosing and were randomized to placebo SC on entry into this maintenance study.
^{b}Subjects who were in clinical response to placebo IV induction dosing and received placebo SC on entry into this maintenance study.
^{c}Subjects who were not in clinical response to guselkumab IV at Week I-12 but were in clinical response at Week I-24 after receiving SC administrations of guselkumab from Week I-12.

TABLE 30
Summary of UC Disease Characteristics at Maintenance Baseline; Full Analysis Set (Study CNTO1959UCO3001; Maintenance)

| | | | | | | Nonrandomized Subjects | | |
|---|---|---|---|---|---|---|---|---|
| | | | | | | | Induction | |
| | | | | | | | Guselkumab | |
| | Randomized Subjects | | | | | Induction | 24-week | |
| | Induction Guselkumab IV Responders | | | | | Placebo IV | Responders^{c} | |
| | | Guselkumab | | | | Responders | Guselkumab | |
| | Placebo | 100 mg SC | 200 mg SC | | | Placebo | 200 mg SC | Overall |
| | SC^{a} | q8w | q4w | Combined | Total | SC^{b} | q4w | Total |
| Analysis set: Full | 190 | 188 | 190 | 378 | 568 | 114 | 123 | 805 |
| Mayo score | | | | | | | | |
| N | 190 | 188 | 190 | 378 | 568 | 114 | 123 | 805 |
| Mean (SD) | 3.3 | 3.2 | 3.2 | 3.2 | 3.2 | 3.8 | 4.3 | 3.5 |
| | (2.01) | (1.91) | (1.85) | (1.88) | (1.92) | (2.08) | (1.81) | (1.97) |
| Median Range | 3.0 | 3.0 | 3.0 | 3.0 | 3.0 | 4.0 | 5.0 | 4.0 |
| | (0; 8) | (0; 7) | (0; 8) | (0; 8) | (0; 8) | (0; 8) | (0; 9) | (0; 9) |
| IQ range | (2.0; | (2.0; | (2.0; | (2.0; | (2.0; | (2.0; | (3.0; | (2.0; |
| | 5.0) | 5.0) | 5.0) | 5.0) | 5.0) | 5.0) | 6.0) | 5.0) |
| Modified Mayo | | | | | | | | |
| score | | | | | | | | |
| N | 190 | 188 | 190 | 378 | 568 | 114 | 123 | 805 |
| Mean (SD) | 2.5 | 2.6 | 2.5 | 2.5 | 2.5 | 2.9 | 3.4 | 2.7 |
| | (1.57) | (1.51) | (1.50) | (1.51) | (1.53) | (1.56) | (1.41) | (1.55) |
| Median Range | 2.0 | 2.0 | 3.0 | 2.0 | 2.0 | 3.0 | 4.0 | 3.0 |
| | (0; 6) | (0; 6) | (0; 6) | (0; 6) | (0; 6) | (0; 6) | (0; 7) | (0; 7) |
| IQ range | (1.0; | (1.0; | (1.0; | (1.0; | (1.0; | (2.0; | (3.0; | (2.0; |
| | 4.0) | 4.0) | 4.0) | 4.0) | 4.0) | 4.0) | 4.0) | 4.0) |
| Partial Mayo | | | | | | | | |
| score | | | | | | | | |
| N | 190 | 188 | 190 | 378 | 568 | 114 | 123 | 805 |
| Mean (SD) | 1.7 | 1.6 | 1.6 | 1.6 | 1.6 | 2.1 | 2.2 | 1.8 |
| | (1.34) | (1.18) | (1.19) | (1.18) | (1.24) | (1.37) | (1.35) | (1.29) |
| Median Range | 2.0 | 2.0 | 2.0 | 2.0 | 2.0 | 2.0 | 2.0 | 2.0 |
| | (0; 5) | (0; 5) | (0; 5) | (0; 5) | (0; 5) | (0; 5) | (0; 6) | (0; 6) |
| IQ range | (1.0; | (1.0; | (1.0; | (1.0; | (1.0; | (1.0; | (1.0; | (1.0; |
| | 3.0) | 2.0) | 2.0) | 2.0) | 2.0) | 3.0) | 3.0) | 3.0) |
| Endoscopic | | | | | | | | |
| healing^{d} | | | | | | | | |
| N | 190 | 188 | 190 | 378 | 568 | 114 | 123 | 805 |
| Yes | 68 | 75 | 79 | 154 | 222 | 39 | 29 | 290 |
| | (35.8%) | (39.9%) | (41.6%) | (40.7%) | (39.1%) | (34.2%) | (23.6%) | (36.0%) |
| No | 122 | 113 | 111 | 224 | 346 | 75 | 94 | 515 |
| | (64.2%) | (60.1%) | (58.4%) | (59.3%) | (60.9%) | (65.8%) | (76.4%) | (64.0%) |
| Endoscopic | | | | | | | | |
| normalization^{e} | | | | | | | | |
| N | 190 | 188 | 190 | 378 | 568 | 114 | 123 | 805 |
| Yes | 39 | 41 | 47 | 88 | 127 | 21 | 12 | 160 |
| | (20.5%) | (21.8%) | (24.7%) | (23.3%) | (22.4%) | (18.4%) | (9.8%) | (19.9%) |
| No | 151 | 147 | 143 | 290 | 441 | 93 | 111 | 645 |
| | (79.5%) | (78.2%) | (75.3%) | (76.7%) | (77.6%) | (81.6%) | (90.2%) | (80.1%) |
| Clinical | | | | | | | | |
| remission^{f} | | | | | | | | |
| N | 190 | 188 | 190 | 378 | 568 | 114 | 123 | 805 |
| Yes | 59 | 66 | 69 | 135 | 194 | 33 | 20 | 247 |
| | (31.1%) | (35.1%) | (36.3%) | (35.7%) | (34.2%) | (28.9%) | (16.3%) | (30.7%) |
| No | 131 | 122 | 121 | 243 | 374 | 81 | 103 | 558 |
| | (68.9%) | (64.9%) | (63.7%) | (64.3%) | (65.8%) | (71.1%) | (83.7%) | (69.3%) |
| IBDQ | | | | | | | | |
| remission^{g} | | | | | | | | |
| N | 188 | 188 | 189 | 377 | 565 | 112 | 123 | 800 |
| Yes | 142 | 134 | 128 | 262 | 404 | 66 | 67 | 537 |
| | (75.5%) | (71.3%) | (67.7%) | (69.5%) | (71.5%) | (58.9%) | (54.5%) | (67.1%) |
| No | 46 | 54 | 61 | 115 | 161 | 46 | 56 | 263 |
| | (24.5%) | (28.7%) | (32.3%) | (30.5%) | (28.5%) | (41.1%) | (45.5%) | (32.9%) |
| CRP (mg/L) | | | | | | | | |
| N | 190 | 188 | 190 | 378 | 568 | 114 | 123 | 805 |
| Mean (SD) | 3.5 | 4.6 | 3.4 | 4.0 | 3.8 | 4.6 | 4.3 | 4.0 |
| | (5.13) | (11.19) | (6.61) | (9.18) | (8.05) | (7.97) | (5.64) | (7.72) |
| Median | 1.5 | 1.4 | 1.4 | 1.4 | 1.5 | 1.6 | 2.0 | 1.6 |
| Range | (0; 32) | (0; 116) | (0; 71) | (0; 116) | (0; 116) | (0; 50) | (0; 32) | (0; 116) |
| IQ range | (0.6; | (0.4; | (0.6; | (0.5; | (0.6; | (0.7; | (0.9; | (0.6; |
| | 4.0) | 4.0) | 3.4) | 3.7) | 3.8) | 5.2) | 6.2) | 4.2) |
| Abnormal | 63 | 63 | 56 | 119 | 182 | 42 | 50 | 274 |
| CRP (>3 mg/L) | (33.2%) | (33.5%) | (29.5%) | (31.5%) | (32.0%) | (36.8%) | (40.7%) | (34.0%) |
| Fecal calprotectin | | | | | | | | |
| (mg/kg) | | | | | | | | |
| N | 188 | 185 | 187 | 372 | 560 | 113 | 122 | 795 |
| Mean | 1143.2 | 1292.3 | 942.4 | 1116.4 | 1125.4 | 1078.0 | 1244.4 | 1136.9 |
| (SD) | (2683.43) | (2953.34) | (1577.16) | (2367.19) | (2475.49) | (1554.96) | (2315.56) | (2339.84) |
| Median | 306.5 | 308.0 | 281.0 | 301.5 | 303.5 | 488.0 | 530.5 | 358.0 |
| Range | (15; 19291) | (15; 27538) | (15; 10708) | (15; 27538) | (15; 27538) | (15; 11027) | (15; 18934) | (15; 27538) |
| IQ range | (82.5; | (71.0; | (89.0; | (78.0; | (79.5; | (91.0; | (186.0; | (88.0; |
| | 1077.0) | 1310.0) | 1233.0) | 1282.0) | 1194.0) | 1488.0) | 1335.0) | 1273.0) |
| Abnormal fecal | 102 | 101 | 97 | 198 | 300 | 68 | 79 | 447 |
| calprotectin | (54.3%) | (54.6%) | (51.9%) | (53.2%) | (53.6%) | (60.2%) | (64.8%) | (56.2%) |
| (>250 mg/kg) | | | | | | | | |
| Fecal | 121 | 116 | 119 | 235 | 356 | 78 | 95 | 529 |
| calprotectin >150 | (64.4%) | (62.7%) | (63.6%) | (63.2%) | (63.6%) | (69.0%) | (77.9%) | (66.5%) |
| mg/kg | | | | | | | | |
| Albumin (g/L) | | | | | | | | |
| N | 190 | 188 | 190 | 378 | 568 | 114 | 123 | 805 |
| Mean | 45.9 | 45.9 | 45.4 | 45.6 | 45.7 | 45.6 | 45.2 | 45.6 |
| (SD) | (3.08) | (3.40) | (3.40) | (3.40) | (3.30) | (3.56) | (3.73) | (3.41) |
| Median | 46.0 | 46.0 | 46.0 | 46.0 | 46.0 | 45.0 | 45.0 | 46.0 |
| Range | (37; 56) | (35; 57) | (37; 57) | (35; 57) | (35; 57) | (36; 55) | (32; 54) | (32; 57) |
| IQ range | (44.0; | (44.0; | (43.0; | (44.0; | (44.0; | (43.0; | (43.0; | (43.0; |
| | 48.0) | 48.0) | 48.0) | 48.0) | 48.0) | 48.0) | 47.0) | 48.0) |

Key: CRP = C-reactive protein; IBDQ = Inflammatory Bowel Disease Questionnaire; IQ = interquartile; SD = standard deviation.
Note:
Includes only subjects with modified Mayo score 5-9 at induction baseline.
Note:
Subjects were presented in the treatment group assigned at Week M-0.
^{a}Subjects who were in clinical response to guselkumab IV induction dosing and were randomized to placebo SC on entry into this maintenance study.
^{b}Subjects who were in clinical response to placebo IV induction dosing and received placebo SC on entry into this maintenance study.
^{c}Subjects who were not in clinical response to guselkumab IV at Week I-12 but were in clinical response at Week I-24 after receiving SC administrations of guselkumab from Week I-12.
^{d}Endoscopic healing is defined as an endoscopy subscore of 0 or 1 with no friability present on the endoscopy.
^{e}Endoscopic normalization is defined as an endoscopy subscore of 0.
^{f}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{g}IBDQ remission is defined as a total IBDQ score ≥ 170.

TABLE 31
Summary of Key Efficacy Endpoints Among Subjects Who Were ADT Non-failure;
Randomized Full Analysis Set (Study CNTO1959UCO3001; Maintenance)

| | | | Guselkumab | | | | | |
|---|---|---|---|---|---|---|---|---|
| | Placebo SC^{a} | | 100 mg SC q8w | | 200 mg SC q4w | | Combined | |
| Analysis set: Randomized Full | 190 | | 188 | | 190 | | 378 | |
| Subjects who were ADT non-failure | 115 | (60.5%) | 111 | (59.0%) | 102 | (53.7%) | 213 | (56.3%) |
| Subjects in clinical remission at Week M-44^{b1,c,d,e} | 30 | (26.1%) | 54 | (48.6%) | 60 | (58.8%) | 114 | (53.5%) |
| 95% CI for treatment proportion^{f} | (18.1%, 34.1%) | | (39.4%, 57.9%) | | (49.3%, 68.4%) | | (46.8% | |
| | | | | | | | 60.2%) | |
| Adjusted treatment difference (95% CI)^{g} | | | 22.2% (10.3%, | | 29.2% (17.4%, | | 25.5% (15.5%, | |
| | | | 34.0%) | | 41.1%) | | 35.6%) | |
| p-value^{h} | | | <0.001 | | <0.001 | | <0.001 | |
| Subjects in symptomatic remission at Week M- | 53 | (46.1%) | 82 | (73.9%) | 78 | (76.5%) | 160 | (75.1%) |
| 44^{b2,c,d,e} | | | | | | | | |
| 95% CI for treatment proportion^{f} | (37.0%, 55.2%) | | (65.7%, 82.0%) | | (68.2%, 84.7%) | | (69.3%, 80.9%) | |
| Adjusted treatment difference (95% CI)^{g} | | | 27.5% (15.3%, | | 28.7% (16.3%, | | 28.2% (17.5%, | |
| | | | 39.6%) | | 41.0%) | | 39.0%) | |
| p-value^{h} | | | <0.001 | | <0.001 | | <0.001 | |
| Subjects with endoscopic healing at Week M- | 30 | (26.1%) | 58 | (52.3%) | 61 | (59.8%) | 119 | (55.9%) |
| 44^{b3,c,d,e} | | | | | | | | |
| 95% CI for treatment proportion^{f} | (18.1%, 34.1%) | | (43.0%, 61.5%) | | (50.3%, 69.3%) | | (49.2%, 62.5%) | |
| Adjusted treatment difference (95% CI)^{g} | | | 25.9% (14.0%, | | 30.3% (18.4%, | | 28.0% (17.9%, | |
| | | | 37.8%) | | 42.3%) | | 38.1%) | |
| p-value^{h} | | | <0.001 | | <0.001 | | <0.001 | |
| Subjects in corticosteroid-free clinical remission | 30 | (26.1%) | 54 | (48.6%) | 58 | (56.9%) | 112 | (52.6%) |
| at Week M-44^{b4,c,d,e} | | | | | | | | |
| 95% CI for treatment proportion^{f} | (18.1%, 34.1%) | | (39.4%, 57.9%) | | (47.3%, 66.5%) | | (45.9%, 59.3%) | |
| Adjusted treatment difference (95% CI)^{g} | | | 22.2% (10.3%, | | 27.2% (15.3%, | | 24.7% (14.6%, | |
| | | | 34.0%) | | 39.1%) | | 34.7%) | |
| p-value^{h} | | | <0.001 | | <0.001 | | <0.001 | |
| Subjects in clinical response at Week M-44^{b5,c,d,e} | 61 | (53.0%) | 92 | (82.9%) | 83 | (81.4%) | 175 | (82.2%) |
| 95% CI for treatment proportion^{f} | (43.9%, 62.2%) | | (75.9%, 89.9%) | | (73.8%, 88.9%) | | (77.0%, 87.3%) | |
| Adjusted treatment difference (95% CI)^{g} | | | 29.7% (18.2%, | | 27.0% (15.1%, | | 28.7% (18.3%, | |
| | | | 41.1%) | | 38.9%) | | 39.1%) | |
| p-value^{h} | | | <0.001 | | <0.001 | | <0.001 | |
| Subjects with histologic-endoscopic mucosal | 26 | (22.6%) | 53 | (47.7%) | 57 | (55.9%) | 110 | (51.6%) |
| healing at Week M-44^{b6,c,d,e} | | | | | | | | |
| 95% CI for treatment proportion^{f} | (15.0%, 30.3%) | | (38.5%, 57.0%) | | (46.2%, 65.5%) | | (44.9%, 58.4%) | |
| Adjusted treatment difference (95% CI)^{g} | | | 24.7% (12.9%, | | 29.7% (18.1%, | | 27.2% (17.3%, | |
| | | | 36.4%) | | 41.4%) | | 37.0%) | |
| p-value^{h} | | | <0.001 | | <0.001 | | <0.001 | |
| Subjects in IBDQ remission at Week M-44^{b7,c,d,e} | 57 | (49.6%) | 76 | (68.5%) | 75 | (73.5%) | 151 | (70.9%) |
| 95% CI for treatment proportion^{f} | (40.4%, 58.7%) | | (59.8%, 77.1%) | | (65.0%, 82.1%) | | (64.8%, 77.0%) | |
| Adjusted treatment difference (95% CI)^{g} | | | 19.0% (6.6%, | | 23.3% (10.9%, | | 21.3% (10.5%, | |
| | | | 31.4%) | | 35.7%) | | 32.2%) | |
| p-value^{h} | | | 0.004 | | <0.001 | | <0.001 | |
| Subjects in fatigue response at Week M-44^{b8,c,d,e} | 42 | (36.5%) | 59 | (53.2%) | 54 | (52.9%) | 113 | (53.1%) |
| 95% CI for treatment proportion^{f} | (27.7%, 45.3%) | | (43.9%, 62.4%) | | (43.3%, 62.6%) | | (46.3%, 59.8%) | |
| Adjusted treatment difference (95% CI)^{g} | | | 16.0% (3.4%, | | 14.9% (2.3%, | | 15.5% (4.6%, | |
| | | | 28.6%) | | 27.6%) | | 26.4%) | |
| p-value^{h} | | | 0.016 | | 0.027 | | 0.007 | |
| Subjects in clinical remission at maintenance | 44 | (38.3%) | 46 | (41.4%) | 51 | (50.0%) | 97 | (45.5%) |
| baseline^{b1,c} | | | | | | | | |
| Subjects in clinical remission at Week M- | 16 | (36.4%) | 28 | (60.9%) | 40 | (78.4%) | 68 | (70.1%) |
| 44^{b1,d,e,i} | | | | | | | | |
| 95% CI for treatment proportion^{j} | (22.4%, 52.2%) | | (45.4%, 74.9%) | | (64.7%, 88.7%) | | (60.0%, 79.0%) | |
| Treatment difference (95% CI)^{j} | | | 24.5% (3.4%, | | 42.1% (22.5%, | | 33.7% (16.1%, | |
| | | | 44.1%) | | 59.1%) | | 50.0%) | |
| p-value^{j} | | | 0.023 | | <0.001 | | <0.001 | |
| Subjects with endoscopic normalization at Week | 23 | (20.0%) | 41 | (36.9%) | 43 | (42.2%) | 84 | (39.4%) |
| M-44^{b9,c,d,e} | | | | | | | | |
| 95% CI for treatment proportion^{f} | (12.7%, 27.3%) | | (28.0%, 45.9%) | | (32.6%, 51.7%) | | (32.9%, 46.0%) | |
| Adjusted treatment difference (95% CI)^{g} | | | 16.6% (5.3%, | | 18.3% (7.3%, | | 17.4% (7.9%, | |
| | | | 28.0%) | | 29.2%) | | 26.9%) | |
| p-value^{h} | | | 0.006 | | 0.002 | | <0.001 | |
| Subjects with histologic healing at Week M- | 44 | (38.3%) | 77 | (69.4%) | 70 | (68.6%) | 147 | (69.0%) |

44^{b10,c,d,e}

| 95% CI for treatment proportion^{f} | (29.4%, 47.1%) | (60.8%, 77.9%) | (59.6%, 77.6%) | (62.8%, 75.2%) |
|---|---|---|---|---|
| Adjusted treatment difference (95% CI)^{g} | | 31.2% (19.0%, | 27.6% (15.1%, | 29.7% (19.0%, |
| | | 43.3%) | 40.1%) | 40.5%) |
| p-value^{h} | | <0.001 | <0.001 | <0.001 |

Note:
Includes only subjects with modified Mayo score 5-9 at induction baseline.
^{a}Subjects who were in clinical response to guselkumab IV induction dosing and were randomized to placebo SC on entry into this maintenance study.
^{b1}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{b2}Symptomatic remission is defined as a stool frequency subscore of 0 or 1 and a rectal bleeding subscore of 0, where the stool frequency subscore has not increased from induction baseline.
^{b3}Endoscopic healing is defined as an endoscopy subscore of 0 or 1 with no friability present on the endoscopy.
^{b4}Corticosteroid-free clinical remission is defined as not requiring any treatment with corticosteroids for at least 8 weeks prior to Week M-44 and also meeting the criteria for clinical remission at Week M-44.
^{b5}Clinical response is defined as a decrease from induction baseline in the modified Mayo score by ≥30% and ≥2 points, with either a ≥1-point decrease from induction baseline in the rectal bleeding subscore or a rectal bleeding subscore of 0 or 1.
^{b6}Histologic-endoscopic mucosal healing is defined as achieving a combination of histologic healing and endoscopic healing. Histologic healing is defined as neutrophil infiltration in <5% of crypts, no crypt destruction, and no erosions, ulcerations or granulation tissue according to the Geboes grading system. Endoscopic healing is defined as an endoscopy subscore of 0 or 1 with no friability present on the endoscopy.
^{b7}IBDQ (Inflammatory Bowel Disease Questionnaire) remission is defined as a total IBDQ score ≥170.
^{b8}Fatigue response is defined as ≥7-point improvement from induction baseline in the PROMIS (Patient-Reported Outcomes Measurement Information System) Fatigue Short Form 7a.
^{b9}Endoscopic normalization is defined as an endoscopy subscore of 0.
^{b10}Histologic healing is defined as neutrophil infiltration in <5% of crypts, no crypt destruction, and no erosions, ulcerations or granulation tissue according to the Geboes grading system.
^{c}Denominator is the number of subjects who were ADT non-failure.
^{d}Intercurrent Event (ICE) Strategies: Subjects who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (ICE 4) prior to Week M-44 were considered not to have achieved any of the key efficacy endpoints shown at Week M-44. For subjects who discontinued study agent due to COVID-19 related reasons (excluding COVID-19 infection) or regional crisis in Russia and Ukraine (ICE 5) prior to Week M-44, their observed values (if available) were used. Subjects who experienced ICE 6 (discontinued study agent due to reasons other than those in ICEs 4 and 5) prior to Week M-44 were considered not to have achieved any of the key efficacy endpoints shown at Week M-44.
^{e}Nonresponder Imputation for Missing Data: Subjects who were missing one or more of the components pertaining to an endpoint at Week M-44 were considered not to have achieved the endpoint. Subjects who had an unevaluable biopsy (i.e., a biopsy that was collected, but could not be assessed due to sample preparation or technical errors) were considered not to have achieved the histology endpoints.
^{f}The confidence intervals for the proportion of subjects meeting the endpoint in each treatment group were based on the normal approximation confidence limits.
^{g}The adjusted treatment difference and confidence intervals were based on the Wald statistic with Cochran-Mantel-Haenszel weight.
^{h}The p-values were based on the Cochran-Mantel-Haenszel (CMH) test, stratified by clinical remission status at maintenance baseline (Yes/No), and induction treatment (guselkumab 400 mg IV, guselkumab 200 mg IV, placebo IV crossover to guselkumab 200 mg IV).
ⁱDenominator is the number of subjects in clinical remission at maintenance baseline who were ADT non-failure.
^{j}The p-values were based on the Fisher's exact test. The confidence intervals were based on the exact confidence limits.

TABLE 32
Summary of Key Efficacy Endpoints Among Subjects Who Were ADT Failure;
Randomized Full Analysis Set (Study CNTO1959UCO3001; Maintenance)

| | | | Guselkumab | | | |
|---|---|---|---|---|---|---|
| | Placebo SC^{a} | | 100 mg SC q8w | 200 mg SC q4w | Combined | |
| Analysis set: Randomized Full | 190 | | 188 | 190 | 378 | |
| Subjects who were ADT failure | 75 | (39.5%) | 77 (41.0%) | 88 (46.3%) | 165 | (43.7%) |
| Subjects in clinical remission at Week M-44^{b1,c,d,e} | 6 | (8.0%) | 31 (40.3%) | 35 (39.8%) | 66 | (40.0%) |
| 95% CI for treatment proportion^{f} | (1.9%, 14.1%) | | (29.3%, 51.2%) | (29.5%, 50.0%) | (32.5%, 47.5%) | |
| Adjusted treatment difference (95% CI)^{g} | | | 30.4% (18.7%, | 32.4% (21.1%, | 31.4% (22.3%, | |
| | | | 42.1%) | 43.7%) | 40.5%) | |
| p-value^{h} | | | <0.001 | <0.001 | <0.001 | |
| Subjects in symptomatic remission at Week M-44^{b2,c,d,e} | 18 | (24.0%) | 50 (64.9%) | 53 (60.2%) | 103 | (62.4%) |
| 95% CI for treatment proportion^{f} | (14.3%, 33.7%) | | (54.3%, 75.6%) | (50.0%, 70.5%) | (55.0%, 69.8%) | |
| Adjusted treatment difference (95% CI)^{g} | | | 39.1% (25.9%, | 36.8% (23.4%, | 37.7% (26.2%, | |
| | | | 52.2%) | 50.2%) | 49.3%) | |
| p-value^{h} | | | <0.001 | <0.001 | <0.001 | |
| Subjects with endoscopic healing at Week M-44^{b3,c,d,e} | 6 | (8.0%) | 35 (45.5%) | 37 (42.0%) | 72 | (43.6%) |
| 95% CI for treatment proportion^{f} | (1.9%, 14.1%) | | (34.3%, 56.6%) | (31.7%, 52.4%) | (36.1%, 51.2%) | |
| Adjusted treatment difference (95% CI)^{g} | | | 35.8% (23.8%, | 34.6% (23.1%, | 35.1% (25.9%, | |
| | | | 47.8%) | 46.0%) | 44.3%) | |
| p-value^{h} | | | <0.001 | <0.001 | <0.001 | |
| Subjects in corticosteroid-free clinical remission at Week | 5 | (6.7%) | 31 (40.3%) | 35 (39.8%) | 66 | (40.0%) |
| M-44^{b4,c,d,e} | | | | | | |
| 95% CI for treatment proportion^{f} | (1.0%, 12.3%) | | (29.3%, 51.2%) | (29.5%, 50.0%) | (32.5%, 47.5%) | |
| Adjusted treatment difference (95% CI)^{g} | | | 32.0% (20.6%, | 33.8% (22.8%, | 32.8% (24.0%, | |
| | | | 43.4%) | 44.9%) | 41.6%) | |
| p-value^{h} | | | <0.001 | <0.001 | <0.001 | |
| Subjects in clinical response at Week M-44^{b5,c,d,e} | 21 | (28.0%) | 54 (70.1%) | 59 (67.0%) | 113 | (68.5%) |
| 95% CI for treatment proportion^{f} | (17.8%, 38.2%) | | (59.9%, 80.4%) | (57.2%, 76.9%) | (61.4%, 75.6%) | |
| Adjusted treatment difference (95% CI)^{g} | | | 40.8% (27.4%, | 39.4% (25.8%, | 40.0% (28.1%, | |
| | | | 54.2%) | 53.0%) | 51.9%) | |
| p-value^{h} | | | <0.001 | <0.001 | <0.001 | |
| Subjects with histologic-endoscopic mucosal healing at | 6 | (8.0%) | 29 (37.7%) | 34 (38.6%) | 63 | (38.2%) |
| Week M-44^{b6,c,d,e} | | | | | | |
| 95% CI for treatment proportion^{f} | (1.9%, 14.1%) | | (26.8%, 48.5%) | (28.5%, 48.8%) | (30.8%, 45.6%) | |
| Adjusted treatment difference (95% CI)^{g} | | | 27.7% (16.0%, | 31.2% (19.8%, | 29.7% (20.6%, | |
| | | | 39.5%) | 42.5%) | 38.7%) | |
| p-value^{h} | | | <0.001 | <0.001 | <0.001 | |
| Subjects in IBDQ remission at Week M-44^{b7,c,d,e} | 14 | (18.7%) | 45 (58.4%) | 47 (53.4%) | 92 | (55.8%) |
| 95% CI for treatment proportion^{f} | (9.8%, 27.5%) | | (47.4%, 69.4%) | (43.0%, 63.8%) | (48.2%, 63.3%) | |
| Adjusted treatment difference (95% CI)^{g} | | | 37.9% (25.7%, | 35.4% (22.7%, | 36.5% (25.9%, | |
| | | | 50.1%) | 48.0%) | 47.1%) | |
| p-value^{h} | | | <0.001 | <0.001 | <0.001 | |
| Subjects in fatigue response at Week M-44^{b8,c,d,e} | 14 | (18.7%) | 36 (46.8%) | 28 (31.8%) | 64 | (38.8%) |
| 95% CI for treatment proportion^{f} | (9.8%, 27.5%) | | (35.6%, 57.9%) | (22.1%, 41.5%) | (31.4%, 46.2%) | |
| Adjusted treatment difference (95% CI)^{g} | | | 26.6% (13.4%, | 13.1% (0.7%, | 19.6% (8.7%, | |
| | | | 39.8%) | 25.6%) | 30.5%) | |
| p-value^{h} | | | <0.001 | 0.057 | 0.002 | |
| Subjects in clinical remission at maintenance baseline^{b1,c} | 15 | (20.0%) | 20 (26.0%) | 18 (20.5%) | 38 | (23.0%) |
| Subjects in clinical remission at Week M-44^{b1,d,e,i} | 4 | (26.7%) | 12 (60.0%) | 10 (55.6%) | 22 | (57.9%) |
| 95% CI for treatment proportion^{j} | (7.8%, 55.1%) | | (36.1%, 80.9%) | (30.8%, 78.5%) | (40.8%, 73.7%) | |
| Treatment difference (95% CI)^{j} | | | 33.3% (−0.9%, | 28.9% (−5.9%, | 31.2% (1.0%, | |
| | | | 62.1%) | 59.0%) | 58.1%) | |
| p-value^{j} | | | 0.087 | 0.158 | 0.066 | |
| Subjects with endoscopic normalization at Week M- | 6 | (8.0%) | 24 (31.2%) | 21 (23.9%) | 45 | (27.3%) |
| 44^{b9,c,d,e} | | | | | | |
| 95% CI for treatment proportion^{f} | (1.9%, 14.1%) | | (20.8%, 41.5%) | (15.0%, 32.8%) | (20.5%, 34.1%) | |
| Adjusted treatment difference (95% CI)^{g} | | | 21.4% (10.0%, | 16.3% (6.4%, | 18.5% (10.1%, | |
| | | | 32.7%) | 26.1%) | 26.9%) | |
| p-value^{h} | | | <0.001 | 0.005 | <0.001 | |
| Subjects with histologic healing at Week M-44^{b10,c,d,e} | 14 | (18.7%) | 45 (58.4%) | 52 (59.1%) | 97 | (58.8%) |

| 95% CI for treatment proportion^{f} | (9.8%, 27.5%) | (47.4%, 69.4%) | (48.8%, 69.4%) | (51.3%, 66.3%) |
|---|---|---|---|---|
| Adjusted treatment difference (95% CI)^{g} | | 38.0% (24.6%, | 40.9% (27.8%, | 39.7% (28.6%, |
| | | 51.5%) | 54.0%) | 50.9%) |
| p-value^{h} | | <0.001 | <0.001 | <0.001 |

Note:
Includes only subjects with modified Mayo score 5-9 at induction baseline.
^{a}Subjects who were in clinical response to guselkumab IV induction dosing and were randomized to placebo SC on entry into this maintenance study.
^{b1}Clinical remission is defined as a stool frequency subscore of 0 or 1, a rectal bleeding subscore of 0, and an endoscopy subscore of 0 or 1 with no friability present on the endoscopy, where the stool frequency subscore has not increased from induction baseline.
^{b2}Symptomatic remission is defined as a stool frequency subscore of 0 or 1 and a rectal bleeding subscore of 0, where the stool frequency subscore has not increased from induction baseline.
^{b3}Endoscopic healing is defined as an endoscopy subscore of 0 or 1 with no friability present on the endoscopy.
^{b4}Corticosteroid-free clinical remission is defined as not requiring any treatment with corticosteroids for at least 8 weeks prior to Week M-44 and also meeting the criteria for clinical remission at Week M-44.
^{b5}Clinical response is defined as a decrease from induction baseline in the modified Mayo score by ≥30% and ≥2 points, with either a ≥1-point decrease from induction baseline in the rectal bleeding subscore or a rectal bleeding subscore of 0 or 1.
^{b6}Histologic-endoscopic mucosal healing is defined as achieving a combination of histologic healing and endoscopic healing. Histologic healing is defined as neutrophil infiltration in <5% of crypts, no crypt destruction, and no erosions, ulcerations or granulation tissue according to the Geboes grading system. Endoscopic healing is defined as an endoscopy subscore of 0 or 1 with no friability present on the endoscopy.
^{b7}IBDQ (Inflammatory Bowel Disease Questionnaire) remission is defined as a total IBDQ score ≥170.
^{b8}Fatigue response is defined as ≥7-point improvement from induction baseline in the PROMIS (Patient-Reported Outcomes Measurement Information System) Fatigue Short Form 7a.
^{b9}Endoscopic normalization is defined as an endoscopy subscore of 0.
^{b10}Histologic healing is defined as neutrophil infiltration in <5% of crypts, no crypt destruction, and no erosions, ulcerations or granulation tissue according to the Geboes grading system.
^{c}Denominator is the number of subjects who were ADT failure.
^{d}Intercurrent Event (ICE) Strategies: Subjects who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (ICE 4) prior to Week M-44 were considered not to have achieved any of the key efficacy endpoints shown at Week M-44. For subjects who discontinued study agent due to COVID-19 related reasons (excluding COVID-19 infection) or regional crisis in Russia and Ukraine (ICE 5) prior to Week M-44, their observed values (if available) were used. Subjects who experienced ICE 6 (discontinued study agent due to reasons other than those in ICEs 4 and 5) prior to Week M-44 were considered not to have achieved any of the key efficacy endpoints shown at Week M-44.
^{e}Nonresponder Imputation for Missing Data: Subjects who were missing one or more of the components pertaining to an endpoint at Week M-44 were considered not to have achieved the endpoint. Subjects who had an unevaluable biopsy (i.e., a biopsy that was collected, but could not be assessed due to sample preparation or technical errors) were considered not to have achieved the histology endpoints.
^{f}The confidence intervals for the proportion of subjects meeting the endpoint in each treatment group were based on the normal approximation confidence limits.
^{g}The adjusted treatment difference and confidence intervals were based on the Wald statistic with Cochran-Mantel-Haenszel weight.
^{h}The p-values were based on the Cochran-Mantel-Haenszel (CMH) test, stratified by clinical remission status at maintenance baseline (Yes/No), and induction treatment (guselkumab 400 mg IV, guselkumab 200 mg IV, placebo IV crossover to guselkumab 200 mg IV).
ⁱDenominator is the number of subjects in clinical remission at maintenance baseline who were ADT failure.
^{j}The p-values were based on the Fisher's exact test. The confidence intervals were based on the exact confidence limits.

TABLE 33
Number of Subjects With Histologic Healing at Week M-44; Randomized
Full Analysis Set (Study CNTO1959UCO3001; Maintenance)

| | | Guselkumab | | |
|---|---|---|---|---|
| | Placebo SC^{a} | 100 mg SC q8w | 200 mg SC q4w | Combined |
| Analysis set: Randomized Full | 190 | 188 | 190 | 378 |
| Week M-44 | | | | |
| N | 190 | 188 | 190 | 378 |
| Subjects with histologic healing^{b,c,d} | 58 (30.5%) | 122 (64.9%) | 122 (64.2%) | 244 (64.6%) |
| 95% CI for treatment proportione | (24.0%, 37.1%) | (58.1%, 71.7%) | (57.4%, 71.0%) | (59.7%, 69.4%) |
| Adjusted treatment difference (95% CI)^{f} | | 33.6% (24.3%, | 32.6% (23.3%, | 33.1% (25.1%, |
| | | 42.9%) | 41.9%) | 41.1%) |
| p-value^{g} | | <0.001 | <0.001 | <0.001 |

Note:
Includes only subjects with modified Mayo score 5-9 at induction baseline.
^{a}Subjects who were in clinical response to guselkumab IV induction dosing and were randomized to placebo SC on entry into this maintenance study.
^{b}Histologic healing is defined as neutrophil infiltration in <5% of crypts, no crypt destruction, and no erosions, ulcerations or granulation tissue according to the Geboes grading system.
^{c}Intercurrent Event (ICE) Strategies: Subjects who had an ostomy or colectomy (ICE 1), a dose adjustment (including a sham dose adjustment) (ICE 2), a prohibited change in UC medications (ICE 3), or discontinued study agent due to lack of efficacy or an AE of worsening of UC (ICE 4) prior to Week M-44 were considered not to have achieved histologic healing at Week M-44. For subjects who discontinued study agent due to COVID-19 related reasons (excluding COVID-19 infection) or regional crisis in Russia and Ukraine (ICE 5) prior to Week M-44, their observed values (if available) were used. Subjects who experienced ICE 6 (discontinued study agent due to reasons other than those in ICEs 4 and 5) prior to Week M-44 were considered not to have achieved histologic healing at Week M-44.
^{d}Nonresponder Imputation for Missing Data: After accounting for ICEs, subjects who had an unevaluable biopsy (i.e., a biopsy that was collected, but could not be assessed due to sample preparation or technical errors) or were missing any of the components pertaining to this endpoint (i.e., assessment of neutrophils in epithelium, crypt destruction, or erosions or ulcerations or granulations) at Week M-44 were considered not to have achieved histologic healing.
^{e}The confidence intervals for the proportion of subjects meeting the endpoint in each treatment group were based on the normal approximation confidence limits.
^{f}The adjusted treatment difference and confidence intervals were based on the Wald statistic with Cochran-Mantel-Haenszel weight.
^{g}The p-values were based on the Cochran-Mantel-Haenszel (CMH) test, stratified by clinical remission status at maintenance baseline (Yes/No), and induction treatment (guselkumab 400 mg IV, guselkumab 200 mg IV, placebo IV crossover to guselkumab 200 mg IV).

TABLE 34
Overall Summary of Treatment-emergent Adverse Events Through Week
M-44 (Up to Dose Adjustment); Randomized Safety Analysis Set

| | Randomized | | Randomized Guselkumab^{b} | | | |
|---|---|---|---|---|---|---|
| | Placebo SC^{a,b} | | 100 mg SC q8w | | 200 mg SC q4w | |
| Analysis set: Randomized Safety | 192^{†} | | 186 | | 190 | |
| Average duration of follow-up | 34.0 | | 40.5 | | 39.2 | |
| (weeks) | | | | | | |
| Average exposure (number of | 8.2 | | 9.9 | | 9.6 | |
| administrations) | | | | | | |
| Subjects who died | 0 | | 0 | | 0 | |
| Subjects with 1 or more: | | | | | | |
| Adverse events | 131 | (68.2%) | 120 | (64.5%) | 133 | (70.0%) |
| Adverse events by maximum | | | | | | |
| intensity: | | | | | | |
| Mild | 57 | (29.7%) | 61 | (32.8%) | 73 | (38.4%) |
| Moderate | 69 | (35.9%) | 49 | (26.3%) | 54 | (28.4%) |
| Severe | 5 | (2.6%) | 10 | (5.4%) | 6 | (3.2%) |
| Serious adverse events | 1 | (0.5%) | 5 | (2.7%) | 12 | (6.3%) |
| Adverse events leading to | 13 | (6.8%) | 7 | (3.8%) | 5 | (2.6%) |
| discontinuation of study agent | | | | | | |
| Infections | 63 | (32.8%) | 59 | (31.7%) | 59 | (31.1%) |

| | | | | | |
|---|---|---|---|---|---|
| Serious infections | 0 | 1 | (0.5%) | 2 | (1.1%) |

^{†}2 participants randomized to GUS 100 mg q8w SC only received PBO at M-0 (discontinued the study treatment prior to their first scheduled GUS dose at Week M-4). They were included in the placebo SC treatment group.
^{a}Subjects who were in clinical response to guselkumab IV induction dosing and were randomized to placebo SC on entry into the Maintenance Study.
^{b}Includes data from Week M-0 up to the time of dose adjustment for subjects who had a dose adjustment or through Week M-44 for those who did not.

The invention can be described with reference to the following numbered embodiments:

1. Use of an antibody specific to IL23 for the treatment ulcerative colitis in a patient, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, and the use results in a clinical response in the patient.

2. The use of embodiment 1, wherein the antibody is administered in an initial dose, a dose about 4 weeks after the initial dose and a dose about 8 weeks after the initial dose.

3. The use of embodiment 2, wherein the initial dose and the doses about 4 weeks after the initial dose and about 8 weeks after the initial dose are 200 mg or 400 mg of the antibody.

4. The use of embodiment 3, wherein the administration is intravenous.

5. The use of embodiment 1, wherein the patient is a responder to the antibody and is identified as meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

6. The use of embodiment 1, wherein the patient is a responder to the antibody and is identified as meeting a clinical endpoint, wherein the clinical endpoint is selected from the group consisting of:
(i) clinical remission defined as a stool frequency subscore of 0 or 1, a rectal bleeding subscore of 0, and an endoscopy subscore of 0 or 1 with no friability present on the endoscopy, wherein the stool frequency subscore has not increased from induction baseline;
(ii) symptomatic remission defined as a stool frequency subscore of 0 or 1 and a rectal bleeding subscore of 0, wherein the stool frequency subscore has not increased from induction baseline;
(iii) endoscopic healing defined as an endoscopy subscore of 0 or 1 with no friability present on the endoscopy;
(iv) histologic-endoscopic mucosal healing, defined as achieving a combination of histologic healing and endoscopic healing, where histologic healing is defined as neutrophil infiltration in <5% of crypts, no crypt destruction, and no erosions, ulcerations or granulation tissue according to the Geboes grading system; and
(v) endoscopic normalization defined as an endoscopy subscore of 0 (which requires that no friability is present).

7. The use of embodiment 1, wherein the patient is a responder to the antibody and is identified as having a biomarker of CRP≤3 mg/L and/or a FeCal≤250 mg/kg.

8. The use of embodiment 4, wherein the antibody is administered in a maintenance dose about every 4 weeks or every 8 weeks after the dose administered about 8 weeks after the initial dose.

9. The use of embodiment 8, wherein the maintenance dose is 100 mg or 200 mg of the antibody.

10. The use of embodiment 5-9, wherein the clinical endpoint(s) is measured about 1, 2, 4, 8, 12, 16, 20, 28, 32, 36, 40, 44 and/or 48 weeks after initial treatment.

11. The use of embodiment 10, wherein the clinical endpoint(s) is measured about 12 weeks after initial treatment.

12. The use of embodiment 10, wherein the clinical endpoint(s) is measured about 44 weeks after initial treatment.

13. The use of embodiment 1, wherein the antibody comprises a light chain variable region amino acid sequence of SEQ ID NO: 8 and a heavy chain variable region amino acid sequence of SEQ ID NO: 7.

14. The use of embodiment 1, wherein the antibody comprises a light chain amino acid sequence of SEQ ID NO: 10 and a heavy chain amino acid sequence of SEQ ID NO: 9.

15. The use of embodiment 13 or 14, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

16. The use of embodiment 12 or 12, wherein the antibody is further administered to the patient.

15. The use of embodiment 14, wherein the antibody is administered subcutaneously at a dose of 100 mg or 200 mg.

16. The use of embodiment 1, wherein the patient is not a responder to the antibody and is identified as not meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

17. The use of embodiment 16, wherein the antibody specific to IL23 is further administered to the patient.

18. The use of embodiment 17, wherein the antibody is administered 12 weeks after initial treatment.

19. The use of embodiment 18, wherein the antibody is administered 12 weeks after initial treatment, 16 weeks after initial treatment and 20 weeks after initial treatment.

20. The use of embodiment 19, wherein the antibody is administered subcutaneously at a dose of 200 mg.

21. The use of embodiment 20, wherein the patient is a responder to the antibody and is identified as meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

22. The use of embodiment 20, wherein the patient is a responder to the antibody and is identified as meeting a clinical endpoint, wherein the clinical endpoint is selected from the group consisting of:
(i) clinical remission defined as a stool frequency subscore of 0 or 1, a rectal bleeding subscore of 0, and an endoscopy subscore of 0 or 1 with no friability present on the endoscopy, wherein the stool frequency subscore has not increased from induction baseline;
(ii) symptomatic remission defined as a stool frequency subscore of 0 or 1 and a rectal bleeding subscore of 0, wherein the stool frequency subscore has not increased from induction baseline;
(iii) endoscopic healing defined as an endoscopy subscore of 0 or 1 with no friability present on the endoscopy;
(iv) histologic-endoscopic mucosal healing, defined as achieving a combination of histologic healing and endoscopic healing, where histologic healing is defined as neutrophil infiltration in <5% of crypts, no crypt destruction, and no erosions, ulcerations or granulation tissue according to the Geboes grading system; and
(v) endoscopic normalization defined as an endoscopy subscore of 0 (which requires that no friability is present).

23. The use of embodiment 21 or 22, wherein the clinical endpoint is measured about 24 weeks or 44 weeks after initial treatment.

24. The use of embodiment 21 or 22, wherein the antibody specific to IL23 is further administered to the patient every 4 weeks or every 8 weeks thereafter.

25. The use of embodiment 16, wherein the antibody comprises a light chain variable region amino acid sequence of SEQ ID NO: 8 and a heavy chain variable region amino acid sequence of SEQ ID NO: 7.

26. The use of embodiment 16, wherein the antibody comprises a light chain amino acid sequence of SEQ ID NO: 10 and a heavy chain amino acid sequence of SEQ ID NO: 9.

27. The use of embodiment 25 or 26, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

28. The use of any of embodiments 1-27, further comprising use of one or more additional drugs used to treat ulcerative colitis.

29. The use of embodiment 28, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

30. The use of embodiment 1, wherein the patient is considered a biologic therapy failure or intolerance for ulcerative colitis (Bio-Failure) prior to treatment with the antibody specific to IL23.

31. The use of embodiment 1, wherein the patient is considered a conventional therapy failure or intolerance for ulcerative colitis (Con-Failure) prior to treatment with the antibody specific to IL23.

32. The use of embodiment 1, wherein the ulcerative colitis is moderately to severely active ulcerative colitis.

33. The use of embodiment 32, wherein the patient has endoscopic evidence of active Crohn's disease prior to administration of the initial dose.

34. The use of embodiment 33, wherein the patient has a modified Mayo score of 5 to 9, inclusive, Mayo rectal bleeding subscore ≥1 and a Mayo endoscopy subscore ≥2 prior to administration of the initial dose.

35. Use of an antibody specific to IL23 for the treatment of moderately to severely active ulcerative colitis in a patient, (i) in an initial intravenous dose of 200 mg or 400 mg, (ii) a 200 mg or 400 mg intravenous dose of the antibody about 4 weeks after the initial dose, and (iii) a 200 mg or 400 mg intravenous dose of the antibody about 8 weeks after the initial dose, wherein the antibody comprises a light chain variable region amino acid sequence of SEQ ID NO: 8 and a heavy chain variable region amino acid sequence of SEQ ID NO: 7 and the patient is a responder to the antibody by being identified as meeting a clinical endpoint about 12 weeks or 44 weeks after the initial dose, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

36. The use of embodiment 35, wherein administration of the antibody specific to IL23 is at a dose of 200 mg or 100 mg about every 4 weeks or 8 weeks after administration of the dose about 8 weeks after the initial dose.

What is claimed is:

1. A method of treating ulcerative colitis in a patient, comprising administering to the patient an antibody specific to IL23, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, and wherein the patient is deemed a responder to the antibody.

2. The method of claim 1, wherein the antibody is administered in an initial dose, a dose about 4 weeks after the initial dose and a dose about 8 weeks after the initial dose.

3. The method of claim 2, wherein the initial dose and the doses about 4 weeks after the initial dose and about 8 weeks after the initial dose are 200 mg or 400 mg of the antibody.

4. The method of claim 3, wherein the antibody is administered intravenously.

5. The method of claim 1, wherein the patient is a responder to the antibody and is identified as meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

6. The method of claim 1, wherein the patient is a responder to the antibody and is identified as meeting a clinical endpoint, wherein the clinical endpoint is selected from the group consisting of:
(i) clinical remission defined as a stool frequency subscore of 0 or 1, a rectal bleeding subscore of 0, and an endoscopy subscore of 0 or 1 with no friability present on the endoscopy, wherein the stool frequency subscore has not increased from induction baseline;
(ii) symptomatic remission defined as a stool frequency subscore of 0 or 1 and a rectal bleeding subscore of 0, wherein the stool frequency subscore has not increased from induction baseline;
(iii) endoscopic healing defined as an endoscopy subscore of 0 or 1 with no friability present on the endoscopy;
(iv) histologic-endoscopic mucosal healing, defined as achieving a combination of histologic healing and endoscopic healing, where histologic healing is defined as neutrophil infiltration in <5% of crypts, no crypt destruction, and no erosions, ulcerations or granulation tissue according to the Geboes grading system; and
(v) endoscopic normalization defined as an endoscopy subscore of 0 (which requires that no friability is present).

7. The method of claim 1, wherein the patient is a responder to the antibody and is identified as having a biomarker of CRP≤3 mg/L and/or a FeCal≤250 mg/kg.

8. The method of claim 4, wherein the antibody is administered in a maintenance dose about every 4 weeks or every 8 weeks after the dose administered about 8 weeks after the initial dose.

9. The method of claim 8, wherein the maintenance dose is 100 mg or 200 mg of the antibody.

10. The method of any of claims 5-9, wherein the clinical endpoint(s) is measured about 1, 2, 4, 8, 12, 16, 20, 28, 32, 36, 40, 44 and/or 48 weeks after initial treatment.

11. The method of claim 10, wherein the clinical endpoint(s) and/or biomarker is measured about 12 weeks after initial treatment.

12. The method of claim 10, wherein the clinical endpoint(s) and/or biomarker is measured about 24 weeks after initial treatment.

13. The method of claim 10, wherein the clinical endpoint(s) and/or biomarker is measured about 44 weeks after initial treatment.

14. The method of claim 4, wherein the antibody comprises a light chain variable region amino acid sequence of SEQ ID NO: 8 and a heavy chain variable region amino acid sequence of SEQ ID NO: 7.

15. The method of claim 4, wherein the antibody comprises a light chain amino acid sequence of SEQ ID NO: 10 and a heavy chain amino acid sequence of SEQ ID NO: 9.

16. The method of claim 14 or 15, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

17. The method of claim 16, wherein the antibody is further administered in a subcutaneous maintenance dose about every 4 weeks or every 8 weeks after the dose administered about 8 weeks after the initial dose, wherein the maintenance dose is 100 mg or 200 mg.

18. The method of claim 1, wherein the patient is not a responder to the antibody and is identified as not meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

19. The method of claim 18, comprising further administering to the patient the antibody specific to IL23.

20. The method of claim 19, wherein the antibody is administered 12 weeks after initial treatment.

21. The method of claim 19, wherein the antibody is administered 12 weeks after initial treatment, 16 weeks after initial treatment and 20 weeks after initial treatment.

22. The method of claim 21, wherein the antibody is administered subcutaneously at a dose of 100 mg or 200 mg of the antibody.

23. The method of claim 22, wherein the patient is a responder to the antibody and is identified as meeting a clinical endpoint, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

24. The method of claim 14, wherein the patient is a responder to the antibody and is identified as meeting a clinical endpoint, wherein the clinical endpoint is selected from the group consisting of:
(i) clinical remission defined as a stool frequency subscore of 0 or 1, a rectal bleeding subscore of 0, and an endoscopy subscore of 0 or 1 with no friability present on the endoscopy, wherein the stool frequency subscore has not increased from induction baseline;
(ii) symptomatic remission defined as a stool frequency subscore of 0 or 1 and a rectal bleeding subscore of 0, wherein the stool frequency subscore has not increased from induction baseline;
(iii) endoscopic healing defined as an endoscopy subscore of 0 or 1 with no friability present on the endoscopy;
(iv) histologic-endoscopic mucosal healing, defined as achieving a combination of histologic healing and endoscopic healing, where histologic healing is defined as neutrophil infiltration in <5% of crypts, no crypt destruction, and no erosions, ulcerations or granulation tissue according to the Geboes grading system; and
(v) endoscopic normalization defined as an endoscopy subscore of 0 (which requires that no friability is present).

25. The method of claim 23 or 24, wherein the clinical endpoint is measured about 24 weeks after initial treatment.

26. The method of claim 23 or 24, wherein the clinical endpoint is measured about 44 weeks after initial treatment.

27. The method of claim 23 or 24, comprising further administering to the patient the antibody specific to IL23 every 4 weeks or every 8 weeks thereafter subcutaneously at a dose of 100 mg or 200 mg of the antibody.

28. The method of claim 27, wherein the antibody comprises a light chain amino acid sequence of SEQ ID NO: 10 and a heavy chain amino acid sequence of SEQ ID NO: 9.

29. The method of claim 27 or 28, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

30. The method of claim 15, further comprising administering to the patient one or more additional drugs used to treat ulcerative colitis.

31. The method of claim 30, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

32. The method of claim 1, wherein the patient is considered a biologic therapy failure or intolerance for ulcerative colitis (Bio-Failure) prior to treatment with the antibody specific to IL23.

33. The method of claim 1, wherein the patient is considered a conventional therapy failure or intolerance for ulcerative colitis (Con-Failure) prior to treatment with the antibody specific to IL23.

34. The method of claim 1, wherein the ulcerative colitis is moderately to severely active ulcerative colitis.

35. The method of claim 34, wherein the patient has endoscopic evidence of active Crohn's disease prior to administration of the initial dose.

36. The method of claim 35, wherein the patient has a modified Mayo score of 5 to 9, inclusive, Mayo rectal bleeding subscore ≥1 and a Mayo endoscopy subscore ≥2 prior to administration of the initial dose.

37. A method of treating moderately to severely active ulcerative colitis in a patient, comprising administering to the patient (i) an initial intravenous dose of 200 mg or 400 mg of an antibody specific to IL23, (ii) a 200 mg or 400 mg intravenous dose of the antibody about 4 weeks after the initial dose, and (iii) a 200 mg or 400 mg intravenous dose of the antibody about 8 weeks after the initial dose, wherein the antibody comprises a light chain variable region amino acid sequence of SEQ ID NO: 8 and a heavy chain variable region amino acid sequence of SEQ ID NO: 7 and the patient is a responder to the antibody by being identified as meeting a clinical endpoint about 12 weeks after the initial dose, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.

38. The method of claim 37, further comprising administering a maintenance dose of the antibody specific to IL23 at a dose of 100 mg or 200 mg about every 4 weeks or 8 weeks after administering the dose about 8 weeks after the initial dose.

39. A method of treating moderately to severely active ulcerative colitis in a patient, comprising administering to the patient (i) an initial intravenous dose of 200 mg or 400 mg of an antibody specific to IL23, (ii) a 200 mg or 400 mg intravenous dose of the antibody about 4 weeks after the initial dose, (iii) a 200 mg or 400 mg intravenous dose of the antibody about 8 weeks after the initial dose, and (iv) a maintenance dose of the antibody specific to IL23 at a dose of 100 ng or 200 mg about every 4 weeks or 8 weeks after administering the dose about 8 weeks after the initial dose, wherein the antibody comprises a light chain variable region amino acid sequence of SEQ ID NO: 8 and a heavy chain variable region amino acid sequence of SEQ ID NO: 7 and the patient is a responder to the antibody by being identified as meeting a clinical endpoint about 12, 24, and/or 44 weeks after the initial dose, wherein the clinical endpoint is clinical response defined as a decrease from induction baseline in the modified Mayo score by ≥30% and ≥2 points, with either a ≥1-point decrease from baseline in the rectal bleeding subscore or a rectal bleeding subscore of 0 or 1.


SEQUENCE LISTING (10 sequences)

SEQ ID NO: 1 - protein, 5 aa, Homo sapiens
NYWIG

SEQ ID NO: 2 - protein, 17 aa, Homo sapiens
IIDPSNSYTRYSPSFQG

SEQ ID NO: 3 - protein, 8 aa, Homo sapiens
WYYKPFDV

SEQ ID NO: 4 - protein, 14 aa, Homo sapiens
TGSSSNIGSGYDVH

SEQ ID NO: 5 - protein, 7 aa, Homo sapiens
GNSKRPS

SEQ ID NO: 6 - protein, 11 aa, Homo sapiens
ASWTDGLSLVV

SEQ ID NO: 7 - protein, 117 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS